(19) United States
(12) Patent Application Publication
(10) Pub. No.: US 2022/0339172 A1
(43) Pub. Date: Oct. 27, 2022

(54) METHODS FOR TREATING CASTRATION-RESISTANT AND CASTRATION-SENSITIVE PROSTATE CANCER

(71) Applicant: Sumitomo Pharma Oncology, Inc., Cambridge, MA (US)

(72) Inventors: Steven L. Warner, Sandy, UT (US); David J. Bearss, Alpine, UT (US); Stephen Patrick Anthony, Heber City, UT (US); Michael Vincent McCullar, Walnut Creek, UT (US)

(21) Appl. No.: 17/547,799

(22) Filed: Dec. 10, 2021

Related U.S. Application Data
(63) Continuation of application No. 16/706,463, filed on Dec. 6, 2019, now abandoned.
(60) Provisional application No. 62/776,985, filed on Dec. 7, 2018.
(60) Provisional application No. 62/909,147, filed on Oct. 1, 2019.
(60) Provisional application No. 62/926,390, filed on Oct. 25, 2019.

(51) Int. Cl. A61K 31/675; A61P 35/04; A61K 9/00

(52) U.S. Cl. CPC A61K 31/675; A61P 35/04; A61K 9/0053

(57) ABSTRACT

Methods of treating castration-resistant and castration-sensitive prostate cancer using a compound having the following structure (I):

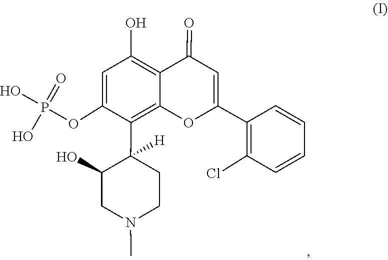 [SMILES: CN1CC[C@H](c2c(OP(=O)(O)O)cc(O)c3c(=O)cc(-c4ccccc4Cl)oc23)[C@H](O)C1]

or a pharmaceutically acceptable salt or zwitterionic form thereof, are provided.

### RELATED APPLICATIONS

This application is a continuation of U.S. application Ser. No. 16/706,463, filed on Dec. 6, 2019, which claims the benefit of U.S. Provisional Application No. 62/926,390, filed on Oct. 25, 2019, U.S. Provisional Application No. 62/909,147, filed on Oct. 1, 2019, and U.S. Provisional Application No. 62/776,985, filed on Dec. 7, 2018. The entire teachings of the above applications are incorporated herein by reference.

### BRIEF SUMMARY

In brief, embodiments of the present invention provide methods of treating castration-resistant prostate cancer. Other embodiments of the present invention provide methods of treating castration-sensitive prostate cancer.

Accordingly, a first embodiment provides a method of treating castration-resistant prostate cancer in a subject in need thereof, comprising administering to the subject an effective amount of a compound having the following structure (I):

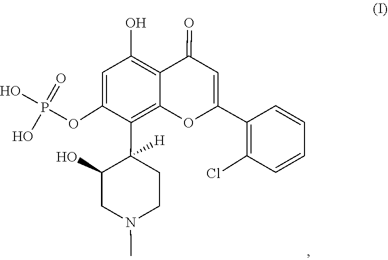 [SMILES: CN1CC[C@H](c2c(OP(=O)(O)O)cc(O)c3c(=O)cc(-c4ccccc4Cl)oc23)[C@H](O)C1]

or a pharmaceutically acceptable salt or zwitterionic form thereof.

A second embodiment provides a method of inhibiting the progression of castration-resistant prostate cancer in a subject in need thereof, comprising administering to the subject an effective amount of a compound having the structure (I), or a pharmaceutically acceptable salt or zwitterionic form thereof.

A third embodiment provides a method inhibiting proliferation of castration-resistant prostate cancer tissue in a subject in need thereof, comprising administering to the subject an effective amount of a compound having the structure (I), or a pharmaceutically acceptable salt or zwitterionic form thereof.

A fourth embodiment provides a method of preventing or inhibiting development of castration-resistant prostate cancer in a subject having prostate cancer, the method comprising administering to the subject an effective amount of a compound having the structure (I), or a pharmaceutically acceptable salt or zwitterionic form thereof.

A fifth embodiment provides a method of treating castration-sensitive prostate cancer in a subject in need thereof, comprising administering to the subject an effective amount of a compound having the structure (I), or a pharmaceutically acceptable salt or zwitterionic form thereof.

A sixth embodiment provides a method of inhibiting the progression of castration-sensitive prostate cancer in a subject in need thereof, comprising administering to the subject an effective amount of a compound having the structure (I), or a pharmaceutically acceptable salt or zwitterionic form thereof.

A seventh embodiment provides a method of inhibiting proliferation of castration-sensitive prostate cancer tissue in a subject in need thereof, comprising administering to the subject an effective amount of a compound having the structure (I), or a pharmaceutically acceptable salt or zwitterionic form thereof.

These and other aspects of embodiments of the invention will be apparent upon reference to the following detailed description. To this end, various references are set forth herein which describe in more detail certain background information, procedures, compounds and/or compositions, and each such reference is hereby incorporated by reference in its entirety.

### BRIEF DESCRIPTION OF THE DRAWINGS

In the figures, identical reference numbers identify similar elements. The sizes and relative positions of elements in the figures are not necessarily drawn to scale and some of these elements are enlarged and positioned to improve figure legibility. Further, the particular shapes of the elements as drawn are not intended to convey any information regarding the actual shape of the particular elements, and have been solely selected for ease of recognition in the figures.

FIGS. 1A-1D show viability assays for prostate cancer cell lines (PC3 in FIG. 1A, VCAP in FIG. 1B, LNCaP in FIG. 1C, and 22Rv1 in FIG. 1D) following treatment with alvocidib, which is the active metabolite of the compound of structure (I) and pharmaceutically acceptable salts and zwitterionic forms thereof.

FIG. 2A shows the effects of alvocidib treatment (3 and 24 hours) on pAR (Ser 515/Ser 81) and ARv7 expression and total AR (TAR) expression in 22Rv1 cells and LNCaP cells following serum stimulation (stimulated 1 hour prior to sample collection).

FIG. 2B shows the effects of alvocidib treatment (24 hours) on pARSer81 ARV7 and ARV7 protein levels in 22Rv1 cells following serum stimulation (stimulated 1 hour prior to sample collection).

FIG. 3 shows the effects of alvocidib treatment (3 and 24 hours) on TMPRSS2 expression in 22Rv1 cells following serum stimulation (stimulated 1 hour prior to sample collection).

FIG. 4 shows the effects of alvocidib treatment (3 and 24 hours) on PSA expression in 22Rv1 cells following serum stimulation (stimulated 23 hours & 3 hours prior to sample collection).

FIG. 5 shows the average tumor volume for each group throughout the 22Rv1 xenograft study.

FIG. 6 shows the tumor volume for each group, as a percentage of the tumor volume of the control group, throughout the 22Rv1 xenograft study.

FIG. 7 shows the average percent change in tumor volume for each group, as a ratio of the average percent change in tumor volume of the control group, throughout the 22Rv1 xenograft study.

FIG. 8 shows the tumor volume for individuals of each group at day 35 of the 22Rv1 xenograft study.

FIG. 9 shows the average percent inhibition of tumor growth for each group, as compared to the control group, throughout the 22Rv1 xenograft study.

FIG. 10 shows the average percent change in inhibition of tumor growth for each group, as compared to the control group, for the 22Rv1 xenograft study.

FIG. 11 shows the average body weight for each group throughout the 22Rv1 xenograft study.

FIG. 12 shows the average percent body weight change for each group throughout the 22Rv1 xenograft study.

FIG. 13 shows the body weight for individuals of groups 1-11 (as defined in Table 1) on day 35 of the 22Rv1 xenograft study.

FIG. 14 shows the percent body weight change at day 35 for individuals of groups 1-11 (as defined in Table 1) of the 22Rv1 xenograft study.

FIG. 15 shows the average tumor volume for each group throughout the C4-2 xenograft study.

FIG. 16 shows the average tumor volume for each group throughout the LNCaP xenograft study.

FIG. 17 shows the average body weight for each group throughout the C4-2 xenograft study.

FIG. 18 shows the average body weight for each group throughout the LNCaP xenograft study.

FIG. 19 shows the effects of alvocidib treatment (3 hours) on RNA Pol II phosphorylation in 22Rv1 cells following serum stimulation.

FIG. 20 shows the effects of alvocidib treatment (48 hours) on PSA protein levels in VCaP and LNCaP cells.

FIG. 21 shows the effects of alvocidib treatment (48 hours) on cell death, as indicated by caspase 3 cleavage, in LNCaP cells.

FIG. 22A shows the plasma concentration (top panel) and tumor concentration (bottom panel) of the compound of structure (I) up to 24 hours after administration of the compound of structure (I) in a PC-3 xenograft model.

FIG. 22B shows that the compound of structure (I) inhibited MCL1 in PC-3 tumors at 4 hours after oral administration, as shown by Western blot.

FIG. 22C shows the effects of the compound of structure (I), administered orally at 1.25 mg/kg BID×21, 7.5 mg/kg q7d×3 or 15 mg/kg q7d×3, on tumor growth in a PC-3 mouse xenograft model.

FIG. 23 is a graph, and shows the completed cycles on the study described in Example 12 through Cohort 5.

FIG. 24A is a graph of plasma alvocidib concentration (ng/mL) versus time, and shows the concentration of alvocidib in the plasma of patients in Cohort 1 on day 1 following daily oral QD dosing with a 1-mg strength capsule containing Formulation No. 401-01.

FIG. 24B is a graph of plasma alvocidib concentration (ng/mL) versus time, and shows the concentration of alvocidib in the plasma of patients in Cohort 1 on day 14 following daily oral QD dosing with a 1-mg strength capsule containing Formulation No. 401-01.

FIG. 24C is a graph of plasma alvocidib concentration (ng/mL) versus time, and shows the concentration of alvocidib in the plasma of patients in Cohort 2 on day 1 following daily oral BID dosing with a 1-mg strength capsule containing Formulation No. 401-01.

FIG. 24D is a graph of plasma alvocidib concentration (ng/mL) versus time, and shows the concentration of alvocidib in the plasma of patients in Cohort 2 on day 14 following daily oral BID dosing with a 1-mg strength capsule containing Formulation No. 401-01.

FIG. 24E is a graph of plasma alvocidib concentration (ng/mL) versus time, and shows the concentration of alvocidib in the plasma of patients in Cohort 5 on day 1 following daily oral BID dosing with 6 mg of Formulation No. 401-01.

FIG. 24F is a graph of plasma alvocidib concentration (ng/mL) versus time, and shows the concentration of alvocidib in the plasma of patients in Cohort 5 on day 14 following daily oral BID dosing with 6 mg of Formulation No. 401-01.

FIG. 24G is a graph of alvocidib (ng/mL) versus cohort, and shows the average C. of alvocidib on day 1 and day 14 following daily oral QD dosing with a 1-mg strength capsule containing Formulation No. 401-01.

FIG. 24H is a graph of alvocidib (ng*hr/mL) versus cohort, and shows the area under the curve (AUC) of alvocidib on day 1 (AUC₀₋₈) and day 14 (AUC₀₋₈ and AUC₀₋₂₄) following daily oral BID dosing with a 1-mg strength capsule containing Formulation No. 401-01.

FIG. 24I is a graph of mean concentration of alvocidib (nM) versus time, and shows the mean concentration of alvocidib in plasma of Cohort 5 patients over a 24-hour period.

FIG. 25 illustrates an x-ray powder diffraction (XRPD) pattern obtained from XRPD analysis of polymorph Form B.

FIG. 26 shows the differential scanning calorimetry output of heat flow plotted as a function of temperature for polymorph Form B.

FIG. 27A is an image of a Western blot, and shows the amount of cleaved caspase 3 as a function of treatment group in the androgen-independent 22Rv1 model described in Example 3.

FIG. 27B is an image of a Western blot, and shows the amount of MCL-1 as a function of treatment group in the androgen-independent 22Rv1 model described in Example 3.

FIG. 27C is an image of a Western blot, and shows the amount of cleaved caspase 3 as a function of treatment group in the androgen-independent 22Rv1 model described in Example 3.

FIG. 27D is an image of a Western blot, and shows the amount of MCL-1 as a function of treatment group in the androgen-independent 22Rv1 model described in Example 3.

FIG. 27E is an image of a Western blot, and shows the amount of C-Myc as a function of treatment group in the 22Rv1 model described in Example 3.

FIG. 27F is a bar graph, and shows the ratios of C-Myc/actin relative to vehicle in the various treatment groups depicted in the Western blot of FIG. 27E.

FIG. 27G is an image of a Western blot, and shows the amount of C-Myc as a function of treatment group in the 22Rv1 model described in Example 3.

FIG. 27H is a bar graph, and shows the ratios of C-Myc/actin relative to vehicle in the various treatment groups depicted in the Western blot of FIG. 27G.

### DETAILED DESCRIPTION

In the following description, certain specific details are set forth in order to provide a thorough understanding of various embodiments of the invention. However, one skilled in the art will understand that the invention may be practiced without these details.

Unless the context requires otherwise, throughout the present specification and claims, the word “comprise” and variations thereof, such as, “comprises” and “comprising” are to be construed in an open, inclusive sense, that is, as “including, but not limited to.”

Reference throughout this specification to “one embodiment” or “an embodiment” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features or characteristics may be combined in any suitable manner in one or more embodiments.

As used herein, the term “about” means±20% (e.g., ±10%, ±5% or ±1%) of the indicated range, value, or structure, unless otherwise indicated.

“Castration-resistant prostate cancer” refers to prostate cancer that progresses in a subject following administration of one or more androgen deprivation therapies (ADTs). Progression of prostate cancer can be evidenced by, for example, a prostate-specific antigen doubling time (PSADT) of less than or equal to 10 months, the progression of pre-existing disease (e.g., radiographic progression, clinical progression, a skeletal-related event, prostate-specific antigen (PSA) progression), and/or the appearance of new metastases in a subject, and is typically driven by androgens, which are a class of hormones including testosterone and dihydrotestosterone (DHT). These androgens bind to the androgen receptor (AR), which is a transcription activator that promotes growth and survival of prostate cells, including prostate cancer cells. ADT refers to a therapy to suppress androgen levels (e.g., surgical castration or chemical castration) or androgen signaling (e.g., by reducing androgen binding to androgen receptor), which may be used to slow the progression of prostate cancer. Androgen deprivation therapy typically causes a temporary reduction in tumor burden concomitant with a decrease in serum PSA. Mechanisms of castration resistance include the emergence of AR variants that are active in the absence of androgen, including splice variants, point mutations to AR, and AR gene amplifications. Castration resistance can be biochemically characterized before the onset of symptoms by a rising titer of serum PSA (Miller, et al., 1992 J. Urol. 147, 956 961). Radiographic progression can be assessed with the use of sequential imaging, and is evidenced by, for example, bone scan identification of two or more new bone lesions with confirmation (according to the Prostate Cancer Clinical Trials Working Group 2 criteria). Response Evaluation Criteria in Solid Tumors (RECIST v 1.1) criteria can also be used to assess radiographic progression of soft tissue lesions. Guidelines for monitoring prostate cancer, including progression of prostate cancer, are described in NCCN Clinical Practice Guidelines in Oncology: Prostate Cancer, version 4.2019, Aug. 19, 2019, the relevant contents of which are incorporated herein by reference in their entirety. “Castration-resistant prostate cancer” is used interchangeably herein with “androgen-resistant prostate cancer”, “androgen-independent prostate cancer” and “hormone-resistant prostate cancer”.

“Castration-sensitive prostate cancer” refers to prostate cancer that does not progress (e.g., responds) following administration of one or more ADTs. Progression of prostate cancer can be assessed according to criteria described herein, for example, with respect to “castration-resistant prostate cancer,” and guidelines for monitoring prostate cancer, including progression of prostate cancer, are described in NCCN Clinical Practice Guidelines in Oncology: Prostate Cancer, version 4.2019, Aug. 19, 2019, the relevant contents of which are incorporated herein by reference in their entirety. “Castration-sensitive prostate cancer” is used interchangeably herein with “androgen-sensitive prostate cancer”, “androgen-dependent prostate cancer” and “hormone-sensitive prostate cancer”.

A “cancer,” including a “tumor,” refers to an uncontrolled growth of cells and/or abnormal increased cell survival and/or inhibition of apoptosis which interferes with the normal functioning of the bodily organs and systems. “Cancer” (e.g., a tumor) includes solid and non-solid cancers. A subject that has a cancer or a tumor has an objectively measurable number of cancer cells present in the subject's body. “Cancers” include benign and malignant cancers (e.g., benign and malignant tumors, respectively), as well as dormant tumors or micrometastases.

A “pharmaceutical composition” refers to a formulation of an active compound, such as a compound of structure (I), or a pharmaceutically acceptable salt or zwitterionic form thereof, and a medium generally accepted in the art for the delivery of the biologically active compound to mammals, e.g., humans. Such a medium includes all pharmaceutically acceptable carriers, diluents or excipients.

An “effective amount” of a pharmaceutical composition according to the invention is a therapeutically effective amount or a prophylactically effective amount.

A “therapeutically effective amount” refers to an amount effective, at dosages and for periods of time necessary, to achieve the desired therapeutic result, such as reduced tumor size (e.g., a 5%, 10%, 15%, or 20% decrease in tumor size), increased life span, a reduction in a prostate cancer biomarker (e.g., a PSA level reduced by 0.1 ng/mL, 0.5 ng/mL, 1 ng/mL, or 5 ng/mL; or a PSA level reduced by at least 10%, at least 15%, at least 20%, at least 25%, at least 30%, at least 40%, or at least 50%), a reduction in a subject's Gleason score (a prostate cancer grading system known by persons of ordinary skill in the art), or increased life expectancy. A therapeutically effective amount of a compound may vary according to factors such as the disease state, age, sex, and weight of the subject, and the ability of the compound to elicit a desired response in the subject. Dosage regimens may be adjusted to provide the optimum therapeutic response. Typically, a therapeutically effective amount is also one in which any toxic or detrimental effects of the compound are outweighed by the therapeutically beneficial effects. In some embodiments, an effective amount is a therapeutically effective amount.

A “prophylactically effective amount” refers to an amount effective, at dosages and for periods of time necessary, to achieve the desired prophylactic result, such as delayed onset of tumors, increased life span, increased life expectancy, preventing or inhibiting the development of castration-resistant prostate cancer, inhibiting the progression of castration-resistant prostate cancer, and/or inhibiting metastasis of prostate cancer. Preventing or inhibiting the development of castration-resistant prostate cancer may be evidenced by a non-castration-resistant prostate cancer (i.e., a cancer that is responsive to androgen deprivation therapy) not progressing to become castration-resistant (i.e., unresponsive to androgen deprivation therapy) or having a delayed progression to castration resistance. Inhibiting the progression of prostate cancer may be evidenced by, for example: no increase in the tumor size, no increase in the Gleason score, no increase in the subject's PSA level, and/or no progression to metastatic castration-resistant prostate cancer (for subjects with non-metastatic castration-resistant prostate cancer). Typically, a prophylactic dose is used in subjects prior to or at an earlier stage of disease (e.g., prior to the cancer becoming castration-resistant), so that a prophylactically effective amount may be less than a therapeutically effective amount. In some embodiments, an effective amount is a prophylactically effective amount.

“Treating” or “treatment” as used herein covers the treatment of the disease or condition of interest in a subject, e.g., a mammal, preferably a human, having the disease or condition of interest, and includes:

inhibiting the disease or condition, e.g., slowing its progression, arresting its development;

(ii) relieving the disease or condition, e.g., causing regression of the disease or condition; and/or

(iii) relieving the symptoms resulting from the disease or condition, e.g., relieving pain without addressing the underlying disease or condition.

As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

A “therapeutic effect,” as used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The terms “co-administration,” “administered in combination with,” and their grammatical equivalents, as used herein, encompass administration of two or more agents to a subject, such as an animal, including humans, to treat a disease, disorder or condition described herein. In some embodiments, administration of the two or more agents is such that both agents and/or their metabolites are present in the subject at the same time. Co-administration includes simultaneous administration in separate compositions, administration at different times in separate compositions, or administration in a composition in which the two or more agents are present.

An “anti-cancer agent,” “anti-tumor agent” or “chemotherapeutic agent” refers to any agent useful in the treatment of a neoplastic condition. One class of anti-cancer agents comprises chemotherapeutic agents. “Chemotherapy” means the administration of one or more chemotherapeutic drugs and/or other agents to a cancer patient by various methods, including intravenous, oral, intramuscular, intraperitoneal, intravesical, subcutaneous, transdermal, buccal, or inhalation or in the form of a suppository.

As used herein, a “subject” refers to an animal. A “subject” may be a mammal, such as a human, non-human primate, rat, mouse, cow, horse, pig, sheep, goat, dog, cat, etc. The subject may be suspected of having or at risk for having castration-resistant prostate cancer. The clinical delineation of castration-resistant prostate cancer is known to those of ordinary skill in the art.

“Mammal” includes humans and both domestic animals such as laboratory animals and household pets (e.g., cats, dogs, swine, cattle, sheep, goats, horses, rabbits), and non-domestic animals such as wildlife and the like.

As used herein, “therapy” refers to any cancer treatment (e.g., chemotherapy, immunotherapy, targeted therapy, hormone therapy, radiation therapy). In some embodiments, a therapy is a chemotherapy.

As used herein, “first-line therapy” refers to the first therapy given for a disease or condition.

As used herein, “subsequent therapy” refers to any therapy given after a first-line therapy for a disease or condition. When a first-line therapy includes drug(s), a subsequent therapy comprises one or more drugs that are different from the drug(s) of a first-line therapy. In some embodiments, the subsequent therapy is a second-line therapy (i.e., the second therapy given for a disease or condition). In some embodiments, the subsequent therapy is a third-line therapy (i.e., the third therapy given for a disease or condition). In some embodiments, the subsequent therapy is a fourth-line therapy (i.e., the fourth therapy given for a disease or condition).

Examples of agents useful in therapies described herein (e.g., as prior therapies, such as a first-line therapy, to therapies comprising one or more of the compounds described herein, such as a compound of structure (I), or a pharmaceutically acceptable salt or zwitterionic form thereof; in combination with one or more of the compounds described herein, such as a compound of structure (I), or a pharmaceutically acceptable salt or zwitterionic form thereof) include darolutamide, apalutamide, enzalutamide, bicalutamide, docetaxel, prednisone, abiraterone (e.g., abiraterone acetate), methylprednisone, radium 223 dichloride (XOFIGO®), an LHRH agonist (e.g., leuprolide, goserelin, triptorelin, histrelin), sipuleucel-T (PROVENGE®), nivolumab, ipilumab, cetrelimab, canerpaturev, PROSTVAC-V, PROSTVAC-F, neoantigen DNA vaccine, paclitaxel (e.g., ABRAXANE®), carboplatin, ramucirumab, mitoxantrone and cabazitaxel, or a pharmaceutically acceptable salt of any of the foregoing, or a combination of two or more of the foregoing. Other agents useful in therapies described herein are described throughout this disclosure.

“Radiation therapy” means exposing a subject, using routine methods and compositions known to the practitioner, to radiation emitters such as alpha-particle emitting radionuclides (e.g., actinium and thorium radionuclides), low linear energy transfer (LET) radiation emitters (i.e., beta emitters), conversion electron emitters (e.g., strontium-89 and samarium-153-EDTMP), or high-energy radiation, including without limitation x-rays, gamma rays, and neutrons.

A subject is said to have “failed” a therapy herein if the subject is diagnosed with castration-resistant prostate cancer following administration of the therapy. For example, a previous treatment with an androgen deprivation therapy may lead to the development of castration-resistant prostate cancer. In such cases, the subject is said to have failed androgen deprivation therapy because the subject is diagnosed with castration-resistant prostate cancer following administration of the androgen deprivation therapy. In another example, a subject previously diagnosed with castration-resistant prostate cancer may be treated with a therapy for castration-resistant prostate cancer, but fail to respond to the treatment. This subject, too, is said to have failed the therapy because the subject is diagnosed with castration-resistant prostate cancer following administration of the therapy.

The term “in vivo” refers to an event that takes place in a subject's body.

Embodiments of the invention disclosed herein are also meant to encompass all pharmaceutically acceptable compounds of structure (I), and pharmaceutically acceptable salts and zwitterionic forms thereof, being isotopically-labelled by having one or more atoms replaced by an atom having a different atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. These radiolabelled compounds could be useful to help determine or measure the effectiveness of the compounds, by characterizing, for example, the site or mode of action, or binding affinity to pharmacologically important site of action. Certain isotopically-labelled compounds of structure (I), and pharmaceutically acceptable salts and zwitterionic forms thereof, for example, those incorporating a radioactive isotope, are useful in drug and/or substrate tissue distribution studies. The radioactive isotopes tritium, i.e., ³H, and carbon-14, i.e., ¹⁴C, are particularly useful for this purpose in view of their ease of incorporation and ready means of detection.

Substitution with heavier isotopes such as deuterium, i.e., ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in positron emission topography (PET) studies for examining substrate receptor occupancy. Isotopically-labeled compounds of structure (I), and pharmaceutically acceptable salts and zwitterionic forms thereof, can generally be prepared by conventional techniques known to those skilled in the art using an appropriate isotopically-labeled reagent in place of the non-labeled reagent previously employed.

“Crystalline,” as used herein, refers to a homogeneous solid formed by a repeating, three-dimensional pattern of atoms, ions or molecules having fixed distances between constituent parts. The unit cell is the simplest repeating unit in this pattern. Notwithstanding the homogenous nature of an ideal crystal, a perfect crystal rarely, if ever, exists. “Crystalline,” as used herein, encompasses crystalline forms that include crystalline defects, for example, crystalline defects commonly formed by manipulating (e.g., preparing, purifying) the crystalline forms described herein. A person skilled in the art is capable of determining whether a sample of a compound is crystalline notwithstanding the presence of such defects.

In some embodiments of the compounds (e.g., crystalline forms) described herein, the compound is substantially pure. As used herein, “substantially pure,” used without further qualification, means the indicated compound has a purity greater than 90 weight percent, for example, greater than 90, 91, 92, 93, 94, 95, 96, 97, 98, or 99 weight percent, and also including a purity equal to about 100 weight percent, based on the weight of the compound. The remaining material comprises other form(s) of the compound, and/or reaction impurities and/or processing impurities arising from its preparation (e.g., alvocidib). Purity can be assessed using techniques known in the art, for example, using an HPLC assay. “Substantially pure” can also be qualified as in “substantially pure of other physical forms of the compound having structure (I), or a pharmaceutically acceptable salt or zwitterionic form thereof,” or “substantially pure of alvocidib.” When qualified thus, “substantially pure” means that the indicated compound contains less than 10%, preferably less than 5%, more preferably less than 3%, most preferably, less than 1% by weight of the indicated impurity (e.g., any other physical forms of an indicated crystalline form of a compound; alvocidib).

As used herein, the term “alvocidib” means 2-(2-chlorophenyl)-5,7-dihydroxy-8-[(3S,4R)-3-hydroxy-1-methylpiperidin-4-yl]chromen-4-one, or a salt (e.g., a pharmaceutically acceptable salt) thereof (e.g., 2-(2-chlorophenyl)-5,7-dihydroxy-8-[(3S,4R)-3-hydroxy-1-methylpiperidin-4-yl]chromen-4-one hydrochloride).

“Polymorph,” as used herein, refers to a crystalline form of a compound characterized by a distinct arrangement of its molecules in a crystal lattice. Polymorphs can be characterized by analytical methods such as x-ray powder diffraction (XRPD), differential scanning calorimetry (DSC) and thermogravimetric analysis.

An XRPD pattern or DSC thermogram that is “substantially in accordance” with one or more figures herein showing an XRPD pattern or diffractogram or DSC thermogram, respectively, is one that would be considered by one skilled in the art to represent the same single crystalline form of the compound having structure (I), or a pharmaceutically acceptable salt or zwitterionic form thereof, as the sample of the compound having structure (I), or a pharmaceutically acceptable salt or zwitterionic form thereof, that provided the pattern or diffractogram or thermogram of one or more figures provided herein. Thus, an XRPD pattern or DSC thermogram that is substantially in accordance may be identical to that of one of the figures or, more likely, may be somewhat different from one or more of the figures. For example, an XRPD pattern that is somewhat different from one or more of the figures may not necessarily show each of the lines of the diffraction pattern presented herein and/or may show a slight change in appearance or intensity of the lines or a shift in the position of the lines. These differences typically result from differences in the conditions involved in obtaining the data or differences in the purity of the sample used to obtain the data. A person skilled in the art is capable of determining if a sample of a crystalline compound is of the same form as or a different form from a form disclosed herein by comparison of the XRPD pattern or DSC thermogram of the sample and the corresponding XRPD pattern or DSC thermogram disclosed herein.

The crystalline forms provided herein can also be identified on the basis of differential scanning calorimetry (DSC) and/or thermogravimetric analysis (TGA). DSC is a thermoanalytical technique in which the difference in the amount of heat required to increase the temperature of a sample is measured as a function of temperature. DSC can be used to detect physical transformations, such as phase transitions, of a sample. For example. DSC can be used to detect the temperature(s) at which a sample undergoes crystallization, melting or glass transition. It is to be understood that any temperature associated with DSC specified herein, with the exception of the DSC temperatures in the Figures or Examples, means the specified value±5° C. or less. For example, when an embodiment or a claim specifies an endothermic peak at 264° C., this is to be understood to mean 264° C.±5° C. or less, that is a temperature of from 259° C. to 269° C. In preferred embodiments, a DSC temperature is the specified value±3° C. or less, in more preferred embodiments, a DSC temperature is the specified value±2° C. or less.

“Pharmaceutically acceptable carrier, diluent or excipient” includes, without limitation, any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

“Zwitterionic form” refers to a form of the compound of structure (I), wherein at least one functional group has a positive electrical charge, at least one functional group has a negative electrical charge, and the net charge of the entire molecule is zero. For example, the phosphate group (—PO₃H₂) of a compound having structure (I) may exist in an anionic form (e.g., —PO₃H⁻), and the nitrogen atom of a compound having structure (I) may exist in the protonated (cationic) form. The compound having structure (II):

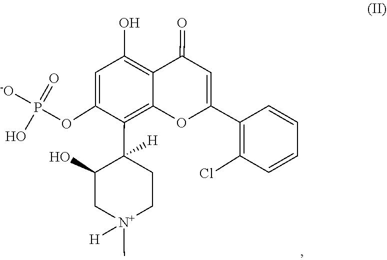

is a zwitterionic form of the compound having structure (I), for example. Embodiments include zwitterions of the compound of structure (I) and the crystalline forms and polymorphs thereof.

A “tautomer” refers to a proton shift from one atom of a molecule to another atom of the same molecule. Embodiments of the present invention include tautomers of the compound of structure (I), even if not specifically illustrated or specified.

### I. Methods

In various embodiments, the invention provides methods for treating castration-resistant prostate cancer in a subject in need thereof by administration of a compound of structure (I), or a pharmaceutically acceptable salt or zwitterionic form thereof, or a pharmaceutical composition comprising the same, to the subject. In other embodiments, the invention provides methods for treating castration-sensitive prostate cancer in a subject in need thereof by administration of a compound of structure (I), or a pharmaceutically acceptable salt or zwitterionic form thereof, or a pharmaceutical composition comprising the same, to the subject.

In a first embodiment, the invention provides a method of treating castration-resistant prostate cancer in a subject in need thereof, the method comprising administering to the subject an effective amount of a compound having the following structure (I):

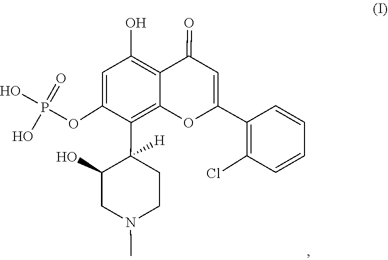 [SMILES: CN1CC[C@H](c2c(OP(=O)(O)O)cc(O)c3c(=O)cc(-c4ccccc4Cl)oc23)[C@H](O)C1]

or a pharmaceutically acceptable salt or zwitterionic form thereof.

In a second embodiment, the invention provides a method of inhibiting the progression of castration-resistant prostate cancer in a subject in need thereof, the method comprising administering to the subject an effective amount of a compound having the following structure (I), or a pharmaceutically acceptable salt or zwitterionic form thereof.

In a third embodiment, the invention provides a method of inhibiting proliferation of castration-resistant prostate cancer tissue in a subject in need thereof, the method comprising administering to the subject an effective amount of a compound having the following structure (I), or a pharmaceutically acceptable salt or zwitterionic form thereof.

In a fourth embodiment, the invention provides a method of treating castration-sensitive prostate cancer in a subject in need thereof, the method comprising administering to the subject an effective amount of a compound having the following structure (I), or a pharmaceutically acceptable salt or zwitterionic form thereof.

In a fifth embodiment, the invention provides a method of inhibiting the progression of castration-sensitive prostate cancer in a subject in need thereof, the method comprising administering to the subject an effective amount of a compound having the following structure (I), or a pharmaceutically acceptable salt or zwitterionic form thereof.

In a sixth embodiment, the invention provides a method of inhibiting proliferation of castration-sensitive prostate cancer tissue in a subject in need thereof, the method comprising administering to the subject an effective amount of a compound having the following structure (I), or a pharmaceutically acceptable salt or zwitterionic form thereof.

In a seventh embodiment, the invention provides a method of preventing or inhibiting development of castration-resistant prostate cancer in a subject having prostate cancer, the method comprising administering to the subject a compound having the following structure (I), or a pharmaceutically acceptable salt or zwitterionic form thereof. Development of castration-resistance may occur, for example, when a subject is treated with an androgen deprivation therapy, by mechanisms such as, for example: alternative splicing of the androgen receptor (e.g., androgen receptor variant 7, which is an active variant that lacks the androgen binding domain), point mutations in the androgen receptor, and/or amplification of the androgen receptor gene.

In some aspects of embodiments one through seven, the subject has been previously administered an androgen deprivation therapy (i.e., prior to the administering of the compound of structure (I), or a pharmaceutically acceptable salt or zwitterionic form thereof). Examples of androgen deprivation therapies include surgical castration, chemical castration (e.g., by treatment with a gonadotropin-releasing hormone (GnRH) agonist, such as leuprorelin, goserelin, triptorelin, histrelin, buserelin; by treatment with a GnRH antagonist, such as degarelix), treatment with an androgen receptor (AR) antagonist and treatment with an androgen receptor signaling inhibitor.

In some aspects of embodiments one through seven, the subject has previously been administered an androgen receptor signaling inhibitor. As used herein, “androgen receptor signaling inhibitor” refers to an agent that inhibits the androgen receptor signaling pathway. Examples of androgen receptor signaling inhibitors include androgen receptor antagonists, such as those described herein. In some embodiments, an androgen receptor signaling inhibitor is abiraterone, apalutamide or enzalutamide.

In some aspects of embodiments one through seven, the subject has previously been administered an androgen receptor (AR) antagonist. Examples of AR antagonists include abiraterone, apalutamide, enzalutamide, flutamide, cyproterone acetate, bicalutamide, nilutamide, ARN-509, AZD-3514, EZN-4176, ODM-201, and TOK-001 (e.g., abiraterone, apalutamide, enzalutamide).

In some aspects of embodiments one through seven, the subject has previously been administered a therapy comprising abiraterone, apalutamide, enzalutamide, flutamide, cyproterone acetate, bicalutamide, nilutamide, ARN-509, AZD-3514, EZN-4176, ODM-201, or TOK-001, or any combination thereof (e.g., abiraterone, apalutamide, enzalutamide).

In some aspects of embodiments one through seven, the compound of structure (I), or a pharmaceutically acceptable salt or zwitterionic form thereof, is administered as a first-line therapy (e.g., as a monotherapy, in combination therapy). In some aspects of embodiments one through seven, the compound of structure (I), or a pharmaceutically acceptable salt or zwitterionic form thereof, is administered as a subsequent therapy (e.g., second-line therapy) after a prior therapy (e.g., a first-line therapy), such as androgen deprivation therapy and/or therapy comprising an androgen receptor signaling inhibitor (e.g., an androgen receptor antagonist, such as enzalutamide, apalutamide or abiraterone), e.g., as a monotherapy, in combination therapy. In some aspects, the subject has failed a prior therapy (e.g., first-line therapy).

In a specific aspect of embodiments one through three, the method is a method of treating metastatic castration-resistant prostate cancer in a subject in need thereof, and comprises administering to the subject an effective amount of a compound having structure (I), or a pharmaceutically acceptable salt or zwitterionic form thereof, wherein the subject has failed a prior therapy (e.g., a first-line therapy) comprising an androgen receptor signaling inhibitor or a taxane. In further aspects, the subject does not have visceral lesions. In yet a further aspect, the compound of structure (I), or a pharmaceutically acceptable salt or zwitterionic form thereof, is administered as a subsequent therapy (e.g., a second-line therapy).

When a compound or agent described herein is described as being administered as a therapy (e.g., a subsequent therapy, prior therapy), it should be understood that the indicated therapy comprises the compound or agent described. For example, when a compound of structure (I), or a pharmaceutically acceptable salt or zwitterionic form thereof, is administered as a subsequent therapy, the subsequent therapy can be a monotherapy involving the compound of structure (I), or a pharmaceutically acceptable salt or zwitterionic form thereof, or a combination therapy involving the compound of structure (I), or a pharmaceutically acceptable salt or zwitterionic form thereof. Stated otherwise, the methods described herein can comprise administering to a subject in need thereof a therapy (e.g., a subsequent, such as second-line, therapy, for example, after a prior therapy) comprising an effective amount of a compound having structure (I), or a pharmaceutically acceptable salt or zwitterionic form thereof.

In certain aspects of the seventh embodiment, the subject has previously been diagnosed with prostate cancer, but has not previously been diagnosed with castration-resistant prostate cancer. For example, a subject may have recently been diagnosed with prostate cancer, and not yet received androgen deprivation therapy. The subject may or may not be simultaneously treated with an androgen deprivation therapy.

In some of any of embodiments one through seven, the compound of structure (I) is provided as a pharmaceutically acceptable salt. In other embodiments, the compound of structure (I) is not a salt, e.g., has structure (I), or a zwitterionic form thereof, and does not include an acid or base counterion. In some embodiments, the compound of structure (I) has the following structure (II):

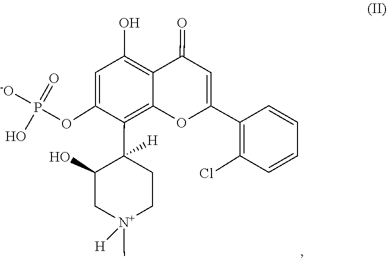

In certain aspects of embodiments one through seven, the subject has an androgen receptor variant (e.g., a predetermined androgen receptor variant) that is associated with castration resistance, such as a point mutation or a splice variant. Examples of androgen receptor point mutations that are associated with prostate cancer becoming castration-resistant include F977L and T878A. Examples of androgen receptor splice variants that are associated with prostate cancer becoming castration-resistant include androgen receptor v7 splice variant, androgen receptor v3 splice variant, androgen receptor v9 splice variant, and androgen receptor v12 splice variant. Exon usage of various splice variants, such as the v7 variant and the v12 variant, can be found, for example, in Dehm, S. & Tindall D., Endocr Relat Cancer. 2011 October; 18(5): R183-R196. Methods of detecting splice variants are generally known by persons of ordinary skill in the art and can be found, for example, in Londono, J., & Philipp, S., BMC Mol Biol. 2016; 17:8 doi: 10.1186/s12867-016-0060-1.

In particular embodiments, the subject has an androgen receptor v7 splice variant.

In aspects of embodiments one through seven, the prostate cancer (e.g., castration-resistant prostate cancer) is metastatic. In other aspects of embodiments one through seven, the prostate cancer (e.g., the castration-resistant prostate cancer) is non-metastatic.

In a certain aspect of embodiments one through seven, the method further includes monitoring the subject's prostate-specific antigen (PSA) level. Steady or reduced PSA levels below an age-dependent threshold (normal PSA levels increase with age) may indicate an effective treatment. When a PSA level remains steady at a low level (e.g., lower than 4.0 ng/mL), this can indicate that a treatment is effective and/or that the prostate cancer is not progressing. If a PSA level stabilizes during treatment (e.g., remains lower than 4.0 ng/mL), and then begins to rise, this may indicate that the prostate cancer has become castration-resistant.

In certain aspects of embodiments one through seven, the method further comprises detecting the subject's PSA level prior to administering the compound having structure (I), or a pharmaceutically acceptable salt or zwitterionic form thereof. In certain aspects of embodiments one through seven, the method further comprises detecting the subject's PSA level after administering the compound having structure (I), or a pharmaceutically acceptable salt or zwitterionic form thereof. In certain aspects of embodiments one through seven, the method further comprises detecting the subject's PSA level prior to administering the compound having structure (I), or a pharmaceutically acceptable salt or zwitterionic form thereof, and after administering the compound having structure (I), or a pharmaceutically acceptable salt or zwitterionic form thereof.

In certain aspects of embodiments one through seven, the subject's PSA level is at least 10% (e.g., at least 15%, at least 20%, at least 25%, at least 30%, at least 40%, at least 50%) lower following administration of the compound having structure (I), or a pharmaceutically acceptable salt or zwitterionic form thereof, than prior to administration of the compound having structure (I), or a pharmaceutically acceptable salt or zwitterionic form thereof.

In some embodiments, the prostate cancer is MCL-1 dependent. As used herein, “MCL-1-dependent” refers to the subset of cancers wherein myeloid cell leukemia 1 (MCL-1) is the primary driver of suppressing apoptosis. Typically, MCL-1 dependency promotes cancer survival, and is associated with treatment resistance and relapse. MCL-1 dependence can be assessed, for example, by contacting a subject's cancer cell with a profiling peptide, as described in International Publication Nos. WO 2016/172214 and WO 2018/119000, the relevant contents of which are incorporated herein by reference in their entireties.

In some embodiments, the cancer is c-Myc-altered. As used herein, “c-Myc-altered” refers to the subset of cancers wherein c-Myc is altered compared to its native sequence, where its expression is amplified compared to an appropriate control (e.g., corresponding normal cells), and where protein levels suggest overexpression of c-Myc. For example, it has been found that c-Myc drives androgen independence in prostate cancer, and overexpression attenuates the anti-tumor activity of androgen receptor suppression. In addition, c-Myc is significantly upregulated in androgen receptor-sensitive prostate cancer. Examples of cancers that can be c-Myc-altered include, but are not limited to, lymphoma (e.g., Burkitt lymphoma, B-cell lymphoma, T-cell lymphoma), cervical cancer, colon cancer, ovarian cancer, breast cancer, lung cancer, prostate cancer, colorectal cancer, pancreatic cancer, gastric cancer and uterine cancer.

In some specific embodiments of all the foregoing methods, the method comprises orally administering the compound of structure (I), or a pharmaceutically acceptable salt or zwitterionic form thereof, or a pharmaceutical composition comprising the same, to the subject.

Some aspects of this invention make use of compositions comprising a compound of structure (I), or a pharmaceutically acceptable salt or zwitterionic form thereof, and pharmaceutically acceptable excipients and/or carriers. Methods described herein include administering a compound of structure (I), or a pharmaceutically acceptable salt or zwitterionic form thereof, as described herein, or a composition (e.g., an effective amount of a composition) of a compound of structure (I), or a pharmaceutically acceptable salt or zwitterionic form thereof, as described herein, or an effective amount of a compound of structure (I), or a pharmaceutically acceptable salt or zwitterionic form thereof, as described herein.

A compound of structure (I) and pharmaceutically acceptable salts and zwitterionic forms thereof can be prepared by addition of a phosphate group to a free hydroxyl of alvocidib, as described in U.S. Patent Publication No.: US 2016/0340376, the full disclosure of which is herein incorporated by reference in its entirety.

It is to be noted that the dosage of the compound of structure (I), or a pharmaceutically acceptable salt or zwitterionic form thereof, may vary in different embodiments. For any particular subject, specific dosage regimens may be adjusted over time according to the individual need and the professional judgement of the person administering or supervising the administration of the compositions. Specific dosages and dosage ranges set forth herein are exemplary only and do not limit the dosages and dosage ranges that may be selected by medical practitioners. The amount of the compound of structure (I), or a pharmaceutically acceptable salt or zwitterionic form thereof, in the composition may vary according to factors such as the disease state, age, sex, and weight of the subject. Dosage regimens may be adjusted to provide the optimum therapeutic response. For example, several divided doses may be administered over time or the dose may be proportionally reduced or increased as indicated by the exigencies of the therapeutic situation.

In some embodiments, the compound of structure (I), or pharmaceutically acceptable salt or zwitterionic form thereof, may be used, for example, and without limitation, in combination with one or more additional therapies for prostate cancer. For example, additional therapies as described herein may be used as neoadjuvant (prior), adjunctive (during), and/or adjuvant (after) therapy with surgery, radiation (brachytherapy or external beam), high-intensity focused ultrasound (HIFU), androgen deprivation (i.e., androgen ablation) or any other therapeutic approach.

In certain aspects of embodiments one through seven, the subject is administered one or more additional therapies. In particular embodiments, the one or more additional therapies is: orchiectomy, radiation, high-beam focused ultrasound (HIFU), and/or one or more additional therapeutic agents with anti-cancer activity.

With respect to combination therapies, one embodiment of the present disclosure provides a combination of any one or more of a compound of structure (I), or pharmaceutically acceptable salt or zwitterionic form thereof, with one or more currently-used or experimental additional therapies which are or may be utilized to treat prostate cancer (e.g., castration-resistant prostate cancer). Methods, uses and pharmaceutical compositions comprising the above combination are also provided.

Accordingly, one embodiment comprises the use of the compound of structure (I), or pharmaceutically acceptable salt or zwitterionic form thereof in combination therapy with one or more pharmacological therapies with anti-cancer activity, irrespective of the biological mechanism of action of such pharmacological therapies, including, without limitation, pharmacological therapies which directly or indirectly inhibit the androgen receptor (e.g., androgen deprivation therapy), pharmacological therapies which are cytotoxic in nature, and pharmacological therapies which interfere with the biological production or function of androgen (hereinafter, the “additional therapeutic agents”). By “combination therapy” is meant the administration of any one or more of a compound of structure (I), or a pharmaceutically acceptable salt or zwitterionic form thereof, and one or more additional therapeutic agents to the same subject. In embodiments, the pharmacological effects of the compound of structure (I), or a pharmaceutically acceptable salt or zwitterionic form thereof, and the one or more additional therapeutic agents are contemporaneous with one another, or if not contemporaneous, synergistic with one another even though dosed sequentially rather than contemporaneously.

Such administration includes, without limitation, dosing of one or more of a compound of structure (I), or pharmaceutically acceptable salt or zwitterionic form thereof, and one or more of the additional therapeutic agent(s) as separate agents without any commingling prior to dosing, as well as formulations which include one or more additional therapeutic agents mixed with one or more compound of structure (I), or pharmaceutically acceptable salt or zwitterionic form thereof, as a pre-mixed formulation. Administration of the compound of structure (I), or pharmaceutically acceptable salt or zwitterionic form thereof, in combination with additional therapeutic agents for treatment of the above disease states also includes dosing by any dosing method including without limitation, intravenous delivery, oral delivery, intra-peritoneal delivery, intra-muscular delivery, or intra-tumoral delivery.

In another aspect of the present disclosure, the one or more of the additional therapeutic agents may be administered to the subject before administration of the compound of structure (I), or pharmaceutically acceptable salt or zwitterionic form thereof. In another embodiment, the compound of structure (I), or pharmaceutically acceptable salt or zwitterionic form thereof, may be co-administered with one or more of the additional therapeutic agents. In yet another aspect, the one or more additional therapeutic agents may be administered to the subject after administration of the compound of structure (I), or pharmaceutically acceptable salt or zwitterionic form thereof.

It is fully within the scope of the disclosure that the ratio of the doses of the compound of structure (I), or pharmaceutically acceptable salts or zwitterionic form thereof, to that of the one or more additional therapeutic agents may or may not equal one and may be varied accordingly to achieve the optimal therapeutic benefit.

The additional therapies include without limitation any pharmacological agent with an anti-cancer effect. For example, the additional therapeutic agent may comprise an alkylating agent, such as chlorambucil, cyclophosphamide, cisplatin; a mitotic inhibitor such as docetaxel (Taxotere; 1,7β,10β-trihydroxy-9-oxo-5β,20-epoxytax-11-ene-2α,4,13α-triyl 4-acetate 2-benzoate 13-{(2R,3 S)-3-[(tert-butoxycarbonyl)amino]-2-hydroxy-3-phenylpropanoate}) or paclitaxel; antimetabolites such as 5-fluorouracil, cytarabine, methotrexate, or pemetrexed; anti-tumor antibiotics such as daunorubicin or doxorubicin; a corticosteroid such as prednisone or methylprednisone; or a Bcl-2 inhibitor such as venetoclax.

In certain aspects of all embodiments (e.g., embodiments four to seven), the additional therapeutic agent is docetaxel. Docetaxel (trade name TAXOTERE®) is a type of chemotherapeutic agent known as an antimicrotubule agent. Docetaxel is used for treating a variety of cancers, such as metastatic prostate cancer. Docetaxel treatment is often administered intravenously, and often includes premedication with a corticosteroid such as prednisone.

In certain aspects of all embodiments (e.g., embodiments one to three and seven), the additional therapeutic agent is venetoclax (GDC-0199, ABT199, RG7601, trade name VENCLEXTA® or VENCLYXTO®), which is a Bcl-2 inhibitor that can induce apoptosis in cancer cells. Venetoclax is typically administered orally.

The additional therapeutic agent may be a pharmacological agent that is currently approved by the Food and Drug Administration (FDA) in the U.S. (or elsewhere by any other regulatory body) for use as pharmacological treatment of prostate cancer, or is currently being used experimentally as part of a clinical trial program that relates to prostate cancer. For example, the additional therapeutic agents may comprise, without limitation, the chemical entity known as enzalutamide or MDV3100 (4-(3-(4-cyano-3-(trifluoromethyl)phenyl)-5,5-dimethyl-4-oxo-2-thioxoimidazolidin-1-yl)-2-fluoro-N-methylbenzamide) and related compounds; the chemical entity known as TOK 001 and related compounds; the chemical entity known as ARN-509; the chemical entity known as abiraterone (or CB-7630; (3S,8R,9S,10R,13S,14S)-10,13-dimethyl-17-(pyridin-3-yl) 2,3,4,7,8,9,10,11,12,13,14,15-dodecahydro-1H-cyclopenta[a]phenanthren-3-ol), and related molecules; the chemical entity known as bicalutamide (N-[4-cyano-3-(trifluoromethyl)phenyl]-3-[(4-fluorophenyl)sulfonyl]-2-hydroxy-2-methylpropanamide) and related compounds; the chemical entity known as nilutamide (5,5-dimethyl-3-[4-nitro-3-(trifluoromethyl)phenyl] imidazolidine-2,4-dione) and related compounds; the chemical entity known as flutamide (2-methyl-N-[4-nitro-3-(trifluoromethyl)phenyl]-propanamide) and related compounds; the chemical entity known as cyproterone acetate (6-chloro-1β,2β-dihydro-17-hydroxy-3′H-cyclopropa[1,2]pregna-4,6-diene-3,20-dione) and related compounds, which is currently used to treat prostate cancer, the chemical entity known as docetaxel and related compounds, which is currently used alone or in combination with prednisone to treat prostate cancer, the chemical entity known as bevacizumab (Avastin), a monoclonal antibody that may be used to treat prostate cancer, the chemical entity known as OSU-HDAC42 ((S)-(+)-N-hydroxy-4-(3-methyl-2-phenylbutyrylamino)-benzamide), and related compounds; the chemical entity known as VITAXIN, which may be used to treat prostate cancer, the chemical entity known as sunitumib (N-(2-diethylaminoethyl)-5-[(Z)-(5-fluoro-2-oxo-1H-indol-3-ylidene)methyl]-2,4-dimethyl-1H-pyrrole-3-carboxamide) and related compounds, which may be used for treatment of prostate cancer, the chemical entity known as ZD-4054 (N-(3-Methoxy-5-methylpyrazin-2-yl)-2-[4-(1,3,4-oxadiazol-2-yl)phenyl]pyridin-3-sulfonamid) and related compounds; the chemical entity known as VN/124-1 (3β-Hydroxy-17-(1H-benzimidazol-1-yl)androsta-5,16-diene), and related compounds; the chemical entity known as cabazitaxel (XRP-6258), and related compounds; the chemical entity known as MDX-010 (Ipilimumab); the chemical entity known as OGX 427; the chemical entity known as OGX 011; the chemical entity known as finasteride (Proscar, Propecia; N-(1,1-dimethylethyl)-3-oxo-(5α,17β)-4-azaandrost-1-ene-17-carboxamide), and related compounds; the chemical entity known as dutasteride (Avodart; 5α, 17β)-N-{2,5 bis(trifluoromethyl) phenyl}-3-oxo-4-azaandrost-1-ene-17-carboxamide) and related compounds; the chemical entity known as turosteride ((4aR,4b S,6aS,7S,9aS,9b S,11aR)-1,4a,6a-trimethyl-2-oxo-N-(propan-2-yl)-N-(propan-2 ylcarbamoyl)hexadecahydro-1H-indeno[5,4-f]quinoline-7-carboxamide), and related compounds; the chemical entity known as bexlosteride (LY-191,704; (4aS,10bR)-8-chloro-4-methyl-1,2,4a,5,6,10b-hexahydrobenzo[f]quinolin-3-one), and related compounds; the chemical entity known as izonsteride (LY-320,236; (4aR,10bR)-8-[(4-ethyl-1,3-benzothiazol-2-yl)sulfanyl]-4,10b-dimethyl-1,4,4a,5,6,10b-hexahydrobenzo[f]quinolin-3(2H)-one) and related compounds; the chemical entity known as FCE 28260 and related compounds; the chemical entity known as SKF105,111, and related compounds; the chemical entity known as AZD3514; the chemical entity known as EZN-4176; the chemical entity known as ODM-201, sipuleucel-T, cabazitaxel; a combination of bevacizumab, docetaxel, thalidomide and prednisone; and/or abiraterone. In certain aspects of all embodiments, the additional therapeutic agent is an androgen receptor antagonist that blocks androgen binding to androgen receptor. Examples of therapies that block androgen binding to androgen receptor include enzalutamide and apalutamide. In particular embodiments, the additional therapeutic agent is enzalutamide. Enzalutamide (trade name XTANDI®) is an androgen receptor (AR) antagonist that is used for treating non-metastatic castration-resistant prostate cancer and metastatic castration-resistant prostate cancer. Enzalutamide treatment may be combined with castration (surgical or chemical).

In certain aspects of all embodiments, the additional therapeutic agent is abiraterone. Abiraterone (trade name ZYTIGA®) is a CYP17A1 inhibitor, which significantly decreases testosterone production. Abiraterone treatment may be combined with other additional therapies, such as a corticosteroid (e.g., prednisone) and/or castration (surgical or chemical).

In certain aspects of all embodiments, the additional therapeutic agent is selected from at least one of: a bromodomain inhibitor, a histone methyltransferase inhibitor, a histone deacetylase inhibitor, or a histone demethylases inhibitor.

In certain aspects of all embodiments, the additional therapeutic agent is a bromodomain inhibitor, for example, an inhibitor of a bromodomain protein such as Brd2, Brd3, Brd4 and/or BrdT. In particular embodiments, the additional therapeutic agent comprises a BRD4 inhibitor. In some of these embodiments, the additional therapeutic agent is JQ-1 (Nature 2010 Dec. 23; 468(7327):1067-73), BI2536 (ACS Chem. Biol. 2014 May 16; 9(5):1160-71; Boehringer Ingelheim), TG101209 (ACS Chem. Biol. 2014 May 16; 9(5):1160-71), OTX015 (Mol. Cancer Ther. November 201312; C244; Oncoethix), IBET762 (J Med Chem. 2013 Oct 10; 56(19):7498-500; GlaxoSmithKline), IBET151 (Bioorg. Med. Chem. Lett. 2012 Apr 15; 22(8):2968-72; GlaxoSmithKline), PFI-1 (J. Med. Chem. 2012 Nov. 26; 55(22):9831-7; Cancer Res. 2013 Jun. 1; 73(11):3336-46; Structural Genomics Consortium) or CPI-0610 (Constellation Pharmaceuticals). In other embodiments, the BRD inhibitor is IBET 762 (GSK525762), TEN-010 (Tensha Therapeutics), CPI-203 (Leukemia. 28 (10): 2049-59, 2014), RVX-208 (Proceedings of the National Academy of Sciences of the United States of America. 110 (49): 19754-9, 2013), LY294002 (ACS Chemical Biology. 9 (2): 495-502, 2014), AZD5153 (Journal of Medicinal Chemistry. 59 (17): 7801-17, 2016), MT-1 (Nature Chemical Biology. 12 (12): 1089-1096 2016) or MS645 (Proceedings of the National Academy of Sciences of the United States of America. 115 (31): 7949-7954, 2018).

In certain aspects of all embodiments, the additional therapeutic agent is a histone methyltransferase inhibitor. In some of these embodiments, the additional therapeutic agent comprises a DOT1-like histone methyltransferase (DOT1L) inhibitor. DOT1L is a histone methyltransferase enzyme that targets lysine 79 in the globular domain of histone H3 for mono-, di-, or trimethylation. In some of these embodiments, the additional therapeutic agent is EPZ004777, EPZ-5676 (Blood. 2013 Aug. 8; 122(6):1017-25) or SGC0946 (Nat. Commun. 2012; 3:1288), for example, EPZ-5676.

In certain aspects of all embodiments, the additional therapeutic agent is a histone deacetylase (HDAC) inhibitor. HDAC proteins may be grouped into classes based on homology to yeast HDAC proteins with Class I made up of HDAC1, HDAC2, HDAC3 and HDAC 8; Class IIa made up of HDAC4, HDAC5, HDAC7 and HDAC 9; Class IIb made up of HDAC6 and HDAC10; and Class IV made up of HDAC11. In some of these embodiments, the additional therapeutic agent is trichostatin A, vorinostat (Proc. Natl. Acad. Sci. U.S.A. 1998 Mar. 17; 95(6):3003-7), givinostat, abexinostat (Mol. Cancer Ther. 2006 May; 5(5):1309-17), belinostat (Mol. Cancer Ther. 2003 August; 2(8):721-8), panobinostat (Clin. Cancer Res. 2006 Aug. 1; 12(15):4628-35), resminostat (Clin. Cancer Res. 2013 Oct 1; 19(19):5494-504), quisinostat (Clin. Cancer Res. 2013 Aug. 1; 19(15):4262-72), depsipeptide (Blood. 2001 Nov. 1; 98(9):2865-8), entinostat (Proc. Natl. Acad. Sci. U.S.A. 1999 Apr 13; 96(8):4592-7), mocetinostat (Bioorg. Med. Chem. Lett. 2008 Feb. 1; 18(3):1067-71) or valproic acid (EMBO J. 2001 Dec 17; 20(24):6969-78). For example, in some embodiments, the additional therapeutic agent is panobinostat. In other embodiments, the additional therapeutic agent is panobinostat or SAHA.

In certain aspects of all embodiments, the additional therapeutic agent is a histone demethylase inhibitor. In particular embodiments, the histone demethylase inhibitor is a lysine-specific demethylase 1A (Lsd1) inhibitor. In some of these embodiments, the additional therapeutic agent is HCI-2509 (BMC Cancer. 2014 Oct 9; 14:752), tranylcypromine or ORY-1001 (J. Clin. Oncol 31, 2013 (suppl; abstr e13543). In other embodiments, the additional therapeutic agent is HCI-2509.

In certain aspects of all embodiments, the additional therapeutic agent is a MLL-menin inhibitor. Menin is a co-factor of the oncogenic MLL fusion protein, and an MLL-menin inhibitor blocks the interaction of the two proteins. Examples of MLL-menin inhibitors include MI-453, M-525, and MI-503.

In certain aspects of all embodiments, the additional therapeutic agent is a B-cell receptor signaling antagonist (e.g., a Bruton's tyrosine kinase (BTK) inhibitor, such as ibrutinib).

In certain aspects of all embodiments, the additional therapeutic agents is an immunomodulator. Immunomodulators of particular interest for use in combination with compounds of the present disclosure include: afutuzumab (available from ROCHE®); pegfilgrastim (NEULASTA®); lenalidomide (CC-5013, REVLIMID®); thalidomide (THALOMID®); actimid (CC4047); and IRX-2 (mixture of human cytokines including interleukin 1, interleukin 2, and interferon γ, CAS 951209-71-5, available from IRX Therapeutics).

In certain aspects of all embodiments, the additional therapeutic agent comprises a chimeric antigen receptor T-cell (CAR-T) therapy. CAR-T therapies of particular interest for use in combination with compounds of the present disclosure include: tisagenlecleucel (Novartis), axicabtagene ciloleucel (Kite), and tocilizumab and atlizumab (Roche).

In certain aspects of all embodiments, the additional therapeutic agent is an immune checkpoint inhibitor (e.g., a PD-1 inhibitor, such as pembrolizumab or nivolumab; a PD-L1 inhibitor, such as atezolizumab, avelumab, or durvalumab; a CTLA-4 inhibitor; a LAG-3 inhibitor; or a Tim-3 inhibitor). Other immune checkpoint inhibitors of interest for use in combination with compounds of the present disclosure include: PD-1 inhibitors, such as pembrolizumab (KEYTRUDA®), nivolumab (OPDIVO®), cemiplimab (LIBTAYO®), spartalizumab (PDR001), pidilizumab (CureTech), MEDI0680 (Medimmune), cemiplimab (REGN2810), dostarlimab (TSR-042), PF-06801591 (Pfizer), tislelizumab (BGB-A317), camrelizumab (INCSHR1210, SHR-1210), and AMP-224 (Amplimmune); PD-L1 inhibitors, such as atezolizumab (TECENTRIQ®), avelumab (BAVENCIO®), durvalumab (IMFINZI®), FAZ053 (Novartis), and BMS-936559 (Bristol-Myers Squibb); and drugs that target CTLA-4, such as ipilimumab (YERVOY®).

In various embodiments, the immune checkpoint inhibitor is a PD-1 inhibitor. In specific embodiments, the PD-1 inhibitor is pembrolizumab, nivolumab, or a combination thereof. In particular embodiments, the PD-1 inhibitor is pembrolizumab (also known as lambrolizumab, MK-3475, MK03475, SCH-900475, or KEYTRUDA®). Pembrolizumab and other anti-PD-1 antibodies are disclosed in Hamid, O., et al. (2013) New England Journal of Medicine 369 (2): 134-44, U.S. Pat. No. 8,354,509, and WO 2009/114335, incorporated by reference in their entireties. In particular embodiments, the PD-1 inhibitor is nivolumab (also known as MDX-1106, MDX-1106-04, ONO-4538, BMS-936558, or OPDIVO®). Nivolumab (clone 5C4) and other anti-PD-1 antibodies are disclosed in U.S. Pat. No. 8,008,449 and WO 2006/121168, incorporated by reference in their entireties. In some other embodiments, the PD-1 inhibitor is AMP-224 (Amplimmune), CBT-501 (CBT Pharmaceuticals), CBT-502 (CBT Pharmaceuticals), JS001 (Junshi Biosciences), IBI308 (Innovent Biologics), INCSHR1210 (Incyte), also known as SHR-1210 (Hengrui Medicine), BGBA317 (Beigene), BGB-108 (Beigene), BAT-I306 (Bio-Thera Solutions), GLS-010 (Gloria Pharmaceuticals; WuXi Biologics), AK103, AK104, AK105 (Akesio Biopharma; Hangzhou Hansi Biologics; Hanzhong Biologics), LZMO09 (Livzon), HLX-10 (Henlius Biotech), MEDI0680 (Medimmune), PDF001 (Novartis), PF-06801591 (Pfizer), pidilizumab (CureTech), REGN2810 (Regeneron), TSR-042 (Tesaro), also known as ANB011, or CS1003 (CStone Pharmaceuticals). MEDI0680 (Medimmune) is also known as AMP-514. MEDI0680 and other anti-PD-1 antibodies are disclosed in U.S. Pat. No. 9,205,148 and WO 2012/145493, incorporated by reference in their entireties. Pidilizumab is also known as CT-011. Pidilizumab and other anti-PD-1 antibodies are disclosed in Rosenblatt, J., et al. (2011) J Immunotherapy 34(5): 409-18, U.S. Pat. Nos. 7,695,715, 7,332,582, and 8,686,119, incorporated by reference in their entireties.

In one embodiment, the anti-PD-1 antibody molecule is cemiplimab. In one embodiment, the anti-PD-1 antibody molecule is sintilimab. In one embodiment, the anti-PD-1 antibody molecule is toripalimab. In one embodiment, the anti-PD-1 antibody molecule is camrelizumab.

Further known anti-PD-1 antibody molecules include those described, e.g., in WO 2015/112800, WO 2016/092419, WO 2015/085847, WO 2014/179664, WO 2014/194302, WO 2014/209804, WO 2015/200119, U.S. Pat. Nos. 8,735,553, 7,488,802, 8,927,697, 8,993,731, and 9,102,727, incorporated by reference in their entireties.

In one embodiment, the PD-1 inhibitor is an anti-PD-1 antibody molecule as described in US 2015/0210769. In one embodiment, the anti-PD-1 antibody molecule comprises the CDRs, variable regions, heavy chains and/or light chains of BAP049-Clone-E or BAP049-Clone-B disclosed in US 2015/0210769. The antibody molecules described herein can be made by vectors, host cells, and methods described in US 2015/0210769, incorporated by reference in its entirety.

In one embodiment, the PD-1 inhibitor is a peptide that inhibits the PD-1 signaling pathway, e.g., as described in U.S. Pat. No. 8,907,053, incorporated by reference in its entirety. In one embodiment, the PD-1 inhibitor is an immunoadhesin (e.g., an immunoadhesin comprising an extracellular or PD-1 binding portion of PD-L1 or PD-L2 fused to a constant region (e.g., an Fc region of an immunoglobulin sequence). In one embodiment, the PD-1 inhibitor is AMP-224 (B7-DCIg (Amplimmune), e.g., disclosed in WO 2010/027827 and WO 2011/066342, incorporated by reference in their entireties).

In some embodiments, the immune checkpoint inhibitor is a PD-L1 inhibitor. In some such embodiments, the PD-L1 inhibitor is atezolizumab, avelumab, durvalumab, or a combination thereof. In particular embodiments, the PD-L1 inhibitor is atezolizumab, also known as MPDL3280A, RG7446, R05541267, YW243.55.570, or TECENTRIQ™. Atezolizumab and other anti-PD-L1 antibodies are disclosed in U.S. Pat. No. 8,217,149, incorporated by reference in its entirety. In particular embodiments, the PD-L1 inhibitor is avelumab, also known as MSB0010718C. Avelumab and other anti-PD-L1 antibodies are disclosed in WO 2013/079174, incorporated by reference in its entirety. In particular embodiments, the PD-L1 inhibitor is durvalumab, also known as MEDI4736. Durvalumab and other anti-PD-L1 antibodies are disclosed in U.S. Pat. No. 8,779,108, incorporated by reference in its entirety. In certain embodiments, the PD-L1 inhibitor is KN035 (Alphamab; 3DMed), BMS 936559 (Bristol-Myers Squibb), CS1001 (CStone Pharmaceuticals), FAZ053 (Novartis), SHR-1316 (Hengrui Medicine), TQB2450 (Chiatai Tianqing), STI-A1014 (Zhaoke Pharm; Lee's Pharm), BGB-A333 (Beigene), MSB2311 (Mabspace Biosciences), or HLX-20 (Henlius Biotech). In one embodiment, the anti-PD-L1 antibody molecule is BMS-936559 (Bristol-Myers Squibb), also known as MDX-1105 or 12A4. BMS-936559 and other anti-PD-L1 antibodies are disclosed in U.S. Pat. No. 7,943,743 and WO 2015/081158, incorporated by reference in their entireties. In some embodiments, the PD-L1 inhibitor is a monoclonal antibody (e.g., as made by Hisun Pharm and applying for clinical trials).

In one embodiment, the PD-L1 inhibitor is an anti-PD-L1 antibody molecule. In one embodiment, the PD-L1 inhibitor is an anti-PD-L1 antibody molecule as disclosed in US 2016/0108123, incorporated by reference in its entirety. In one embodiment, the anti-PD-L1 antibody molecule comprises the CDRs, variable regions, heavy chains and/or light chains of BAP058-Clone 0 or BAP058-Clone N disclosed in US 2016/0108123.

Further known anti-PD-L1 antibodies include those described, e.g., in WO 2015/181342, WO 2014/100079, WO 2016/000619, WO 2014/022758, WO 2014/055897, WO 2015/061668, WO 2013/079174, WO 2012/145493, WO 2015/112805, WO 2015/109124, WO 2015/195163, U.S. Pat. Nos. 8,168,179, 8,552,154, 8,460,927, and 9,175,082, incorporated by reference in their entireties.

In some embodiments, the immune checkpoint inhibitor is a CTLA-4 inhibitor. In certain embodiments, the CTLA-4 inhibitor is ipilimumab. In other embodiments, the CTLA4 inhibitor is tremelimumab.

In some embodiments, the immune checkpoint inhibitor is a LAG-3 inhibitor. In some embodiments, the LAG-3 inhibitor is chosen from LAG525 (Novartis), BMS-986016 (Bristol-Myers Squibb), or TSR-033 (Tesaro). In one embodiment, the LAG-3 inhibitor is an anti-LAG-3 antibody molecule. In one embodiment, the LAG-3 inhibitor is an anti-LAG-3 antibody molecule as disclosed in US 2015/0259420, incorporated by reference in its entirety. In one embodiment, the anti-LAG-3 antibody molecule comprises the CDRs, variable regions, heavy chains and/or light chains of BAP050-Clone I or BAP050-Clone J disclosed in US 2015/0259420.

In one embodiment, the anti-LAG-3 antibody molecule is BMS-986016 (Bristol-Myers Squibb), also known as BMS986016. BMS-986016 and other anti-LAG-3 antibodies are disclosed in WO 2015/116539 and U.S. Pat. No. 9,505,839, incorporated by reference in their entireties. In one embodiment, the anti-LAG-3 antibody molecule is TSR-033 (Tesaro). In one embodiment, the anti-LAG-3 antibody molecule is IMP731 or GSK2831781 (GSK and Prima BioMed). IMP731 and other anti-LAG-3 antibodies are disclosed in WO 2008/132601 and U.S. Pat. No. 9,244,059, incorporated by reference in their entireties. In one embodiment, the anti-LAG-3 antibody molecule is IMP761 (Prima BioMed).

Further known anti-LAG-3 antibodies include those described, e.g., in WO 2008/132601, WO 2010/019570, WO 2014/140180, WO 2015/116539, WO 2015/200119, WO 2016/028672, U.S. Pat. Nos. 9,244,059, 9,505,839, incorporated by reference in their entireties.

In one embodiment, the anti-LAG-3 inhibitor is a soluble LAG-3 protein, e.g., IMP321 (Prima BioMed), e.g., as disclosed in WO 2009/044273, incorporated by reference in its entirety.

In some embodiments, the immune checkpoint inhibitor is a TIM-3 inhibitor. In some embodiments, the TIM-3 inhibitor is MGB453 (Novartis) or TSR-022 (Tesaro).

In one embodiment, the TIM-3 inhibitor is an anti-TIM-3 antibody molecule. In one embodiment, the TIM-3 inhibitor is an anti-TIM-3 antibody molecule as disclosed in US 2015/0218274, incorporated by reference in its entirety. In one embodiment, the anti-TIM-3 antibody molecule comprises the CDRs, variable regions, heavy chains and/or light chains of ABTIM3-hum11 or ABTIM3-hum03 disclosed in US 2015/0218274.

In one embodiment, the anti-TIM-3 antibody molecule is TSR-022 (AnaptysBio/Tesaro). In one embodiment, the anti-TIM-3 antibody molecule comprises one or more of the CDR sequences (or collectively all of the CDR sequences), the heavy chain or light chain variable region sequence, or the heavy chain or light chain sequence of APE5137 or APE5121. APE5137, APE5121, and other anti-TIM-3 antibodies are disclosed in WO 2016/161270, incorporated by reference in its entirety. In one embodiment, the anti-TIM-3 antibody molecule is the antibody clone F38-2E2.

Further known anti-TIM-3 antibodies include those described, e.g., in WO 2016/111947, WO 2016/071448, WO 2016/144803, U.S. Pat. Nos. 8,552,156, 8,841,418, and 9,163,087, incorporated by reference in their entireties.

In an effort to protect normal cells from treatment toxicity and to limit organ toxicities, cytoprotective agents (such as neuroprotectants, free-radical scavengers, cardioprotectors, anthracycline extravasation neutralizers, nutrients and the like) may be used as an adjunct therapy in combination with compounds of the present disclosure. Suitable cytoprotective agents include amifostine (ETHYOL®), glutamine, dimesna (TAVOCEPT®), mesna (MESNEX®), dexrazoxane (ZINECARD® or TOTECT®), xaliproden (XAPRILA®), and leucovorin (also known as calcium leucovorin, citrovorum factor and folinic acid).

Some patients may experience allergic reactions to compounds of the present disclosure and/or other therapeutic agent(s) (e.g., anti-cancer agent(s)) during or after administration.

Therefore, anti-allergic agents can be administered in combination with compounds of the present disclosure and/or other therapeutic agent(s) (e.g., anti-cancer agent(s)) to minimize the risk of an allergic reaction. Suitable anti-allergic agents include corticosteroids (Knutson, S., et al., PLoS One, DOI:10.1371/journal.pone.0111840 (2014)), such as dexamethasone (e.g., DECADRON®), beclomethasone (e.g., BECLOVENT®), hydrocortisone (also known as cortisone, hydrocortisone sodium succinate, hydrocortisone sodium phosphate, sold under the tradenames ALA-CORT®, hydrocortisone phosphate, SOLU-CORTEF®, HYDROCORT ACETATE® and LANACORT®), prednisolone (sold under the tradenames DELTA-CORTEL®, ORAPRED®, PEDIAPRED® and PRELONE®), prednisone (sold under the tradenames DELTASONE®, LIQUID RED®, METICORTEN® and ORASONE®), methylprednisolone (also known as 6-methylprednisolone, methylprednisolone acetate, methylprednisolone sodium succinate, sold under the tradenames DURALONE®, MEDRALONE®, MEDROL®, M-PREDNISOL® and SOLU-MEDROL®); antihistamines, such as diphenhydramine (e.g., BENADRYL®), hydroxyzine, and cyproheptadine; and bronchodilators, such as the beta-adrenergic receptor agonists, albuterol (e.g., PROVENTIL®), and terbutaline (BRETHINE®).

Some patients may experience nausea during and after administration of the compounds described herein and/or other therapeutic agent(s) (e.g., anti-cancer agent(s)). Therefore, anti-emetics can be used in combination with compounds of the present disclosure and/or other therapeutic agent(s) (e.g., anti-cancer agent(s)) to prevent nausea (upper stomach) and vomiting. Suitable anti-emetics include aprepitant (EMEND®), ondansetron (ZOFRAN®), granisetron HCl (KYTRIL®), lorazepam (ATIVAN®, dexamethasone (DECADRON®), prochlorperazine (COMPAZINE®), casopitant (REZONIC® and ZUNRISA®), and combinations thereof.

Medication to alleviate the pain experienced during treatment is often prescribed to make the patient more comfortable. Common over-the-counter analgesics, such TYLENOL®, can also be used in combination with compounds of the present disclosure and/or other therapeutic agent(s) (e.g., anti-cancer agent(s)). Opioid analgesic drugs such as hydrocodone/paracetamol or hydrocodone/acetaminophen (e.g., VICODIN®), morphine (e.g., ASTRAIVIORPH® or AVINZA®), oxycodone (e.g., OXYCONTIN® or PERCOCET®), oxymorphone hydrochloride (OPANA®), and fentanyl (e.g., DURAGESIC®) can be useful for moderate or severe pain, and can be used in combination with compounds of the present disclosure and/or other therapeutic agent(s) (e.g., anti-cancer agent(s)).

In certain aspects of the seventh embodiment, treating a subject having prostate cancer with a compound of structure (I), or a pharmaceutically acceptable salt or zwitterionic form thereof, may prevent or inhibit development of castration-resistance, if the subject is also undergoing an androgen deprivation therapy, or receiving an androgen receptor antagonist that blocks androgen binding to the androgen receptor.

In general, the compound of structure (I), or pharmaceutically acceptable salt or zwitterionic form thereof, should be used without causing substantial toxicity. Toxicity of the compound of structure (I), or pharmaceutically acceptable salt or zwitterionic form thereof, can be determined using standard techniques, for example, by testing in cell cultures or experimental animals and determining the therapeutic index, i.e., the ratio between the LD50 (the dose lethal to 50% of the population) and the LD100 (the dose lethal to 100% of the population). In some circumstances however, such as in severe disease conditions, it may be necessary to administer substantial excesses of the compositions. Titration studies may be used to determine toxic and non-toxic concentrations. Toxicity may be evaluated by examining a particular compound's or composition's specificity across cell lines. Animal studies may be used to provide an indication if the compound has any effects on other tissues.

The compound of structure (I), or pharmaceutically acceptable salt or zwitterionic form thereof, is effective over a wide dosage range. For example, in the treatment of adult humans, dosages from about 0.01 mg to about 1000 mg, from about 0.1 mg to about 100 mg, from about 0.5 mg to about 50 mg per day, and from about 1 mg to about 10 mg per day are examples of dosages that are used in some embodiments. An exemplary dosage is about 0.5 mg to about 50 mg per day. In particular embodiments, the dosage ranges from about 1 mg to about 60 mg (e.g., from about 5 mg to about 60 mg, from about 10 mg to about 60 mg, from about 5 mg to about 50 mg, from about 10 mg to about 30 mg, from about 10 mg to about 50 mg, from about 20 to about 50 mg, from about 25 mg to about 45 mg) per day. In other embodiments, the dosage is from about 1 mg to about 30 mg per day, e.g., about 1 mg, about 2 mg, about 4 mg, about 8 mg, about 12 mg, about 16 mg, about 20 mg, about 22 mg, about 24 mg, about 26 mg, about 28 mg or about 30 mg per day (e.g., administered QD, administered BID). In other embodiments, the dosage is from about 1 mg to about 30 mg, e.g., about 1 mg, about 2 mg, about 4 mg, about 6 mg, about 8 mg, about 11 mg, about 12 mg, about 16 mg, about 20 mg, about 22 mg, about 24 mg, about 26 mg, about 28 mg or about 30 mg, administered BID. The exact dosage will depend upon the route of administration, the form in which the compound of structure (I), or pharmaceutically acceptable salt or zwitterionic form thereof, is administered, the subject to be treated, the body weight of the subject to be treated, and the preference and experience of the attending physician. In some aspects of embodiments one through seven, the therapeutically effective amount is about 0.5 mg to about 50 mg per day. In some aspects of embodiments one through seven, the therapeutically effective amount is about 1 mg to about 60 mg (e.g., from about 5 mg to about 60 mg, from about 10 mg to about 60 mg, from about 5 mg to about 50 mg, from about 10 mg to about 30 mg, from about 10 mg to about 50 mg, from about 20 to about 50 mg, from about 25 mg to about 45 mg) per day. In some aspects of embodiments one through seven, the therapeutically effective amount is from about 1 mg to about 30 mg per day, e.g., about 1 mg, about 2 mg, about 4 mg, about 8 mg, about 12 mg, about 16 mg, about 20 mg, about 22 mg, about 24 mg, about 26 mg, about 28 mg or about 30 mg per day (e.g., administered QD, administered BID). In some aspects of embodiments one through seven, the therapeutically effective amount is from about 1 mg to about 30 mg, e.g., about 1 mg, about 2 mg, about 4 mg, about 6 mg, about 8 mg, about 11 mg, about 12 mg, about 16 mg, about 20 mg, about 22 mg, about 24 mg, about 26 mg, about 28 mg or about 30 mg, administered BID.

In some embodiments, a compound of structure (I), or a pharmaceutically acceptable salt or zwitterionic form thereof, is administered in a single dose. A single dose of a compound of structure (I), or a pharmaceutically acceptable salt or zwitterionic form thereof, may also be used for treatment of an acute condition.

In some embodiments, a compound of structure (I), or a pharmaceutically acceptable salt or zwitterionic form thereof, is administered in multiple doses. In some embodiments, dosing is about once, twice, three times, four times, five times, six times, or more than six times per day.

In certain particular embodiments, the dosing is twice per day (BID). In certain particular embodiments, the dosing is once per day (QD). In other embodiments, dosing is about once a month, once every two weeks, once a week, or once every other day. In another embodiment a compound of structure (I), or a pharmaceutically acceptable salt or zwitterionic form thereof, and another agent are administered together about once per day to about 6 times per day. In another embodiment, the administration of a compound of structure (I), or a pharmaceutically acceptable salt or zwitterionic form thereof, and an agent continues for less than about 7 days. In yet another embodiment the administration continues for more than about 6, 10, 14, 21, 28 days, two months, six months, or one year. In some cases, continuous dosing is achieved and maintained as long as necessary (e.g., until progression or unacceptable toxicity).

Administration of the compound of structure (I), or a pharmaceutically acceptable salt or zwitterionic form thereof, may continue as long as necessary. In some embodiments, a compound of structure (I), or a pharmaceutically acceptable salt or zwitterionic form thereof, is administered for more than 1, 2, 3, 4, 5, 6, 7, 14, or 28 days. In some embodiments, a compound of structure (I), or a pharmaceutically acceptable salt or zwitterionic form thereof, is administered for less than 28, 14, 7, 6, 5, 4, 3, 2, or 1 day. In some embodiments, a compound of structure (I), or a pharmaceutically acceptable salt or zwitterionic form thereof, is administered for 21 consecutive days. In some embodiments, a compound of structure (I), or a pharmaceutically acceptable salt or zwitterionic form thereof, is administered chronically on an ongoing basis, e.g., for the treatment of chronic effects.

Administration of the compound of structure (I), or a pharmaceutically acceptable salt or zwitterionic form thereof, may be performed on a treatment cycle. As used herein, “treatment cycle” refers to a period of treatment followed by a period of no treatment intended to be repeated on a regular schedule. In some embodiments, the treatment cycle is a 21-day treatment cycle. In some embodiments, the treatment cycle is a 28-day treatment cycle.

Administration of the compound of structure (I), or a pharmaceutically acceptable salt or zwitterionic form thereof, may include continuous dosing and/or may include treatment interruptions. For a dosing schedule including treatment interruptions, the compound of structure (I), or a pharmaceutically acceptable salt or zwitterionic form thereof, may be administered on a treatment cycle including a time period of continuous dosing, followed by a treatment interruption wherein the compound is not administered. The treatment interruption may be, for example, more than 1, 2, 3, 4, 5, 6, 7, 14, or 28 days. In certain particular embodiments, the dosing schedule is a 21-day treatment cycle including 14 days of dosing, followed by a treatment interruption of 7 days. In other particular embodiments, the dosing schedule is a 28-day treatment cycle including 21 days of dosing (e.g., BID dosing, QD dosing), followed by a treatment interruption of 7 days. Stated otherwise, in some embodiments, the compound having structure (I), or a pharmaceutically acceptable salt or zwitterionic form thereof, is administered on the first 21 days of a 28-day treatment cycle, and is not administered on days 22 to 28 of the 28-day treatment cycle. The treatment cycles may be repeated at least once, at least twice, at least three times, or at least four times.

In some embodiments, the compound of structure (I), or a pharmaceutically acceptable salt or zwitterionic form thereof, is administered in dosages. Due to intersubject variability in compound pharmacokinetics, individualization of dosing regimen is provided in certain embodiments. Dosing for a compound of structure (I), or a pharmaceutically acceptable salt or zwitterionic form thereof, may be found by routine experimentation in light of the instant disclosure and/or can be derived by one of ordinary skill in the art.

Other examples of cancers treatable according to the methods described herein include hematologic cancers. Hematologic malignancies that can be treated with a compound having structure (I), or a tautomer or zwitterionic form thereof, include leukemias and lymphomas. In some embodiments, the hematologic cancer is selected from acute myelogenous leukemia (AML), follicular lymphoma, acute lymphoblastic leukemia (ALL), chronic lymphocytic leukemia (CLL), multiple myeloma (MM) and non-Hodgkin's lymphoma (e.g., AML, follicular lymphoma, ALL, CLL and non-Hodgkin's lymphoma). In more specific embodiments, the hematological cancer is AML. In other more specific embodiments, the hematologic cancer is CLL. In more specific embodiments, the hematologic cancer is MM. In still other specific embodiments, the hematologic cancer is myelodysplasic syndrome (MDS).

Solid tumors can also be treated according to the methods described herein. Accordingly, in some embodiments, the cancer is a solid tumor cancer. In various embodiments, the solid tumor cancer is breast cancer, bladder cancer, liver cancer, pancreatic cancer, lung cancer, colorectal cancer, ovarian cancer, prostate cancer, or melanoma. In some embodiments, the cancer is bladder cancer. In some embodiments, the cancer is lung cancer. In other embodiments, the cancer is liver cancer. In various embodiments, the solid tumor cancer is breast cancer, bladder cancer, liver cancer, pancreatic cancer, lung cancer, colorectal cancer, ovarian cancer, prostate cancer, or melanoma. In some embodiments, the cancer is bladder cancer. In some embodiments, the cancer is lung cancer. In other embodiments, the cancer is liver cancer. In some embodiments, the cancer is a sarcoma, bladder cancer or renal cancer. In some embodiments, the cancer is prostate cancer. In other embodiments, the cancer is bladder cancer, pancreatic cancer, colorectal cancer, kidney cancer, non-small cell lung carcinoma, prostate cancer, sarcoma, skin cancer, thyroid cancer, testicular cancer or vulvar cancer. In some embodiments, the cancer is endometrial cancer, pancreatic cancer, testicular cancer, renal cancer, melanoma, colorectal cancer, thyroid cancer, bladder cancer, pancreatic cancer, vulvar cancer, sarcoma, prostate cancer, lung cancer or anal cancer.

Further examples of cancers treatable according to the methods described herein include, but are not limited to, Acute Lymphoblastic Leukemia (ALL); Acute Myeloid Leukemia (AML); Adrenocortical Carcinoma; Adrenocortical Carcinoma, Childhood; AIDS-Related Cancer (e.g., Kaposi Sarcoma, AIDS-Related Lymphoma, Primary CNS Lymphoma); Anal Cancer; Appendix Cancer; Astrocytomas, Childhood; Atypical Teratoid/Rhabdoid Tumor, Childhood, Central Nervous System; Basal Cell Carcinoma of the Skin; Bile Duct Cancer; Bladder Cancer; Bladder Cancer, Childhood; Bone Cancer (including Ewing Sarcoma, Osteosarcoma and Malignant Fibrous Histiocytoma); Brain Tumors/Cancer; Breast Cancer; Burkitt Lymphoma; Carcinoid Tumor (Gastrointestinal); Carcinoid Tumor, Childhood; Cardiac (Heart) Tumors, Childhood;

Embryonal Tumors, Childhood; Germ Cell Tumor, Childhood; Primary CNS Lymphoma; Cervical Cancer; Childhood Cervical Cancer; Cholangiocarcinoma; Chordoma, Childhood; Chronic Lymphocytic Leukemia (CLL); Chronic Myelogenous Leukemia (CML); Chronic Myeloproliferative Neoplasms; Colorectal Cancer; Childhood Colorectal Cancer; Craniopharyngioma, Childhood; Cutaneous T-Cell Lymphoma (e.g., Mycosis Fungoides and Sézary Syndrome); Ductal Carcinoma In Situ (DCIS); Embryonal Tumors, Central Nervous System, Childhood; Endometrial Cancer (Uterine Cancer); Ependymoma, Childhood; Esophageal Cancer; Childhood Esophageal Cancer; Esthesioneuroblastoma; Ewing Sarcoma; Extracranial Germ Cell Tumor, Childhood; Extragonadal Germ Cell Tumor; Eye Cancer; Childhood Intraocular Melanoma; Intraocular Melanoma; Retinoblastoma; Fallopian Tube Cancer; Fibrous Histiocytoma of Bone, Malignant, and Osteosarcoma; Gallbladder Cancer; Gastric (Stomach) Cancer; Childhood Gastric (Stomach) Cancer; Gastrointestinal Carcinoid Tumor; Gastrointestinal Stromal Tumors (GIST); Childhood Gastrointestinal Stromal Tumors; Germ Cell Tumors; Childhood Central Nervous System Germ Cell Tumors (e.g., Childhood Extracranial Germ Cell Tumors, Extragonadal Germ Cell Tumors, Ovarian Germ Cell Tumors, Testicular Cancer); Gestational Trophoblastic Disease; Hairy Cell Leukemia; Head and Neck Cancer; Heart Tumors, Childhood; Hepatocellular (Liver) Cancer; Histiocytosis, Langerhans Cell; Hodgkin Lymphoma; Hypopharyngeal Cancer; Intraocular Melanoma; Childhood Intraocular Melanoma; Islet Cell Tumors, Pancreatic Neuroendocrine Tumors; Kaposi Sarcoma; Kidney (Renal Cell) Cancer; Langerhans Cell Histiocytosis; Laryngeal Cancer; Leukemia; Lip and Oral Cavity Cancer; Liver Cancer; Lung Cancer (Non-Small Cell and Small Cell); Childhood Lung Cancer; Lymphoma; Male Breast Cancer; Malignant Fibrous Histiocytoma of Bone and Osteosarcoma; Melanoma; Childhood Melanoma; Melanoma, Intraocular (Eye); Childhood Intraocular Melanoma; Merkel Cell Carcinoma; Mesothelioma, Malignant; Childhood Mesothelioma; Metastatic Cancer; Metastatic Squamous Neck Cancer with Occult Primary;

Midline Tract Carcinoma With NUT Gene Changes; Mouth Cancer; Multiple Endocrine Neoplasia Syndromes; Multiple Myeloma/Plasma Cell Neoplasms; Mycosis Fungoides; Myelodysplastic Syndromes, Myelodysplastic/Myeloproliferative Neoplasms; Myelogenous Leukemia, Chronic (CIVIL); Myeloid Leukemia, Acute (AML); Myeloproliferative Neoplasms, Chronic; Nasal Cavity and Paranasal Sinus Cancer; Nasopharyngeal Cancer; Neuroblastoma; Non-Hodgkin Lymphoma; Non-Small Cell Lung Cancer; Oral Cancer, Lip and Oral Cavity Cancer and Oropharyngeal Cancer; Osteosarcoma and Malignant Fibrous Histiocytoma of Bone; Ovarian Cancer; Childhood Ovarian Cancer; Pancreatic Cancer; Childhood Pancreatic Cancer; Pancreatic Neuroendocrine Tumors; Papillomatosis (Childhood Laryngeal); Paraganglioma;

Childhood Paraganglioma; Paranasal Sinus and Nasal Cavity Cancer; Parathyroid Cancer; Penile Cancer; Pharyngeal Cancer; Pheochromocytoma; Childhood Pheochromocytoma; Pituitary Tumor; Plasma Cell Neoplasm/Multiple Myeloma; Pleuropulmonary Blastoma; Pregnancy and Breast Cancer; Primary Central Nervous System (CNS) Lymphoma; Primary Peritoneal Cancer; Prostate Cancer; Rectal Cancer; Recurrent Cancer; Renal Cell (Kidney) Cancer; Retinoblastoma;

Rhabdomyosarcoma, Childhood; Salivary Gland Cancer; Sarcoma (e.g., Childhood Rhabdomyosarcoma, Childhood Vascular Tumors, Ewing Sarcoma, Kaposi Sarcoma, Osteosarcoma (Bone Cancer), Soft Tissue Sarcoma, Uterine Sarcoma); Sézary Syndrome; Skin Cancer; Childhood Skin Cancer; Small Cell Lung Cancer; Small Intestine Cancer; Soft Tissue Sarcoma; Squamous Cell Carcinoma of the Skin; Squamous Neck Cancer with Occult Primary, Metastatic; Stomach (Gastric) Cancer; Childhood Stomach (Gastric) Cancer; T-Cell Lymphoma, Cutaneous (e.g., Mycosis Fungoides and Sezary Syndrome); Testicular Cancer; Childhood Testicular Cancer; Throat Cancer (e.g., Nasopharyngeal Cancer, Oropharyngeal Cancer, Hypopharyngeal Cancer); Thymoma and Thymic Carcinoma; Thyroid Cancer; Transitional Cell Cancer of the Renal Pelvis and Ureter; Ureter and Renal Pelvis, Transitional Cell Cancer;

Urethral Cancer; Uterine Cancer, Endometrial; Uterine Sarcoma; Vaginal Cancer; Childhood Vaginal Cancer; Vascular Tumors; Vulvar Cancer; and Wilms Tumor and Other Childhood Kidney Tumors.

Metastases of the aforementioned cancers can also be treated in accordance with the methods described herein. Thus, in some embodiments, the cancer is a metastatic cancer. In other embodiments, the cancer is a non-metastatic cancer.

### II. Crystalline and Polymorph Forms of Compounds of Structure (I)

It has been found that compounds having structure (I), or a tautomer or zwitterionic form thereof, can exist in various crystalline and/or polymorphic forms.

Accordingly, one embodiment provides a crystalline form of a compound having the following structure (I):

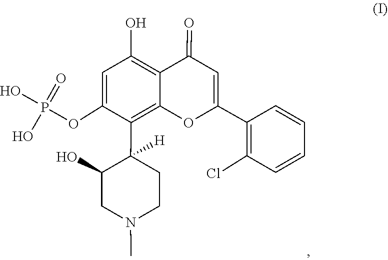 [SMILES: CN1CC[C@H](c2c(OP(=O)(O)O)cc(O)c3c(=O)cc(-c4ccccc4Cl)oc23)[C@H](O)C1]

or a tautomer or zwitterionic form thereof. In some embodiments, the crystalline form comprises Form B. In some embodiments, the crystalline form consists essentially of Form B. In some embodiments, the crystalline form consists of Form B. In some embodiments, the crystalline form is of a compound having structure (II).

Form B has structure (II):

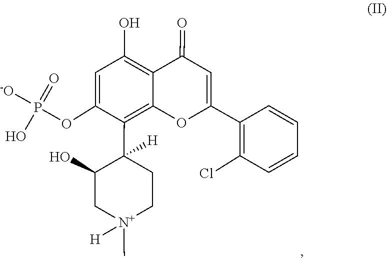

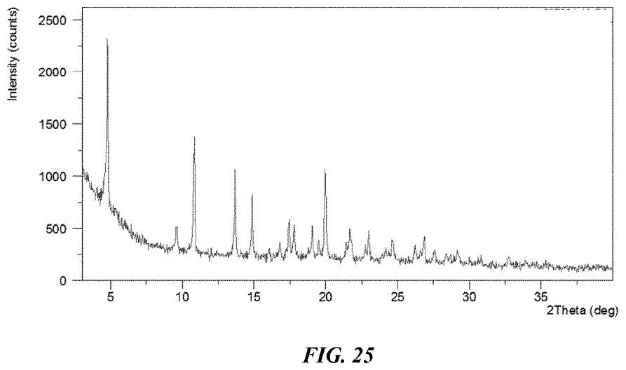

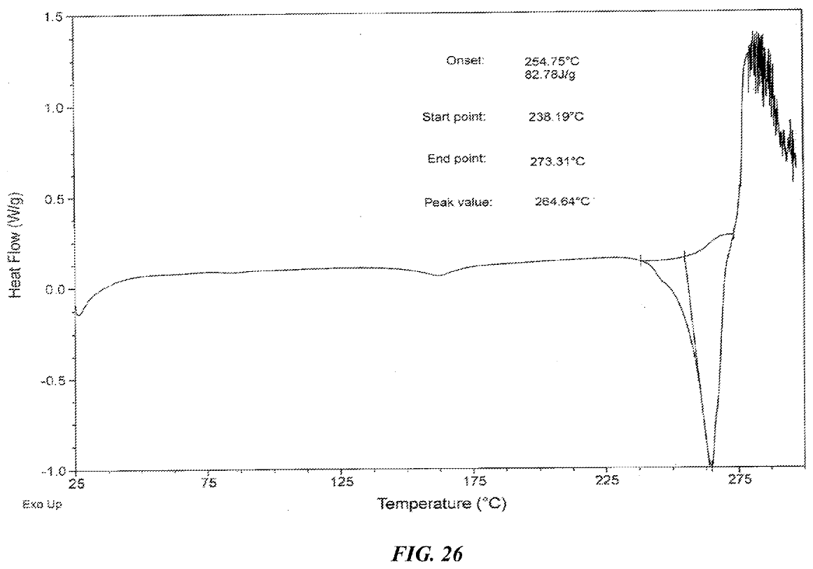

and is characterized, in some embodiments, by an x-ray powder diffraction (XRPD) pattern comprising at least three peaks (e.g., three peaks, at least four peaks, four peaks, at least five peaks, five peaks, six peaks) at 2-theta angles selected from the group consisting of 4.8±0.2°, 10.8±0.2°, 13.7±0.2°, 14.9±0.2°, 20.0±0.2° and 24.6±0.2°. In some embodiments, Form B is characterized by an XRPD pattern comprising peaks at the following 2-theta angles: 10.8±0.2°, 14.9±0.2° and 20.0±0.2°. In some embodiments, Form B is characterized by an XRPD pattern comprising peaks at the following 2-theta angles: 4.8±0.2°, 10.8±0.2°, 14.9±0.2° and 20.0±0.2°. In some embodiments, Form B is characterized by an XRPD pattern comprising peaks at the following 2-theta angles: 4.8±0.2°, 10.8±0.2°, 13.7±0.2°, 14.9±0.2° and 20.0±0.2°. In some embodiments, Form B has an XRPD pattern substantially in accordance with that depicted in FIG. 25. In some embodiments, Form B is characterized by a DSC thermogram comprising an endothermic peak at about 264° C. In some embodiments, Form B is characterized by a DSC thermogram substantially in accordance with that depicted in FIG. 26.

### III. Pharmaceutical Compositions

For the purposes of administration, the compound of structure (I), or a pharmaceutically acceptable salt or zwitterionic form thereof, may be administered as a raw chemical or may be formulated as pharmaceutical compositions. Pharmaceutical compositions provided in the methods described herein comprise a compound of structure (I), or a pharmaceutically acceptable salt or zwitterionic form thereof, and a pharmaceutically acceptable carrier, diluent or excipient. In embodiments, the compound of structure (I), or pharmaceutically acceptable salts or zwitterionic form thereof, is present in the composition in an amount which is effective to treat castration resistant prostate cancer, and preferably with acceptable toxicity to the patient. Bioavailability of compounds of structure (I), or pharmaceutically acceptable salts or zwitterionic form thereof, can be determined by one skilled in the art, for example, as described in the Examples below. Appropriate concentrations and dosages can be readily determined by one skilled in the art.

Administration of the compound of structure (I), or a pharmaceutically acceptable salt or zwitterionic form thereof, in pure form or in an appropriate pharmaceutical composition, can be carried out via any of the accepted modes of administration of agents for serving similar utilities. The pharmaceutical compositions of embodiments of the invention can be prepared by combining a compound of structure (I), or a pharmaceutically acceptable salt or zwitterionic form thereof, with an appropriate pharmaceutically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. Typical routes of administering such pharmaceutical compositions include, without limitation, oral, topical, transdermal, inhalation, parenteral, sublingual, buccal, rectal, vaginal, and intranasal. The term parenteral as used herein includes subcutaneous injections, intravenous, intramuscular, intrasternal injection or infusion techniques. Pharmaceutical compositions of the invention are formulated so as to allow the active ingredients contained therein to be bioavailable upon administration of the composition to a patient. Compositions that will be administered to a subject or patient take the form of one or more dosage units, where for example, a tablet may be a single dosage unit. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will, in any event, contain a therapeutically effective amount of a compound of structure (I), or a pharmaceutically acceptable salt or zwitterionic form thereof, for treatment of castration resistant prostate cancer in accordance with the teachings of this invention. In certain aspects of all embodiments described herein, the compound of structure (I), or pharmaceutically acceptable salts or zwitterionic form thereof, is administered orally.

A pharmaceutical composition of some embodiments of the invention may be in the form of a solid or liquid. In one aspect, the carrier(s) are particulate, so that the compositions are, for example, in tablet or powder form. The carrier(s) may be liquid, with the compositions being, for example, an oral syrup, injectable liquid or an aerosol, which is useful in, for example, inhalatory administration.

When intended for oral administration, the pharmaceutical composition is preferably in either solid or liquid form, where semi-solid, semi-liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like form. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent.

When the pharmaceutical composition is in the form of a capsule, for example, a plant-based capsule such as a hydroxypropyl methylcellulose (HPMC) capsule or a gelatin capsule, it may contain, in addition to materials of the above type, a liquid carrier such as polyethylene glycol or oil.

The pharmaceutical composition may be in the form of a liquid, for example, an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid pharmaceutical compositions of some embodiments of the invention, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid pharmaceutical composition of certain embodiments of the invention intended for either parenteral or oral administration should contain an amount of a compound of structure (I), or a pharmaceutically acceptable salt thereof, such that a suitable dosage will be obtained.

In some embodiments, the pharmaceutical composition of embodiments of the invention may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a pharmaceutical composition for topical administration. If intended for transdermal administration, the composition may include a transdermal patch or iontophoresis device.

The pharmaceutical composition of various embodiments of the invention may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter and polyethylene glycol.

Embodiments of the pharmaceutical composition of the invention may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a capsule, such as an HPMC capsule.

The pharmaceutical composition of some embodiments of the invention in solid or liquid form may include an agent that binds to the compound of structure (I), or a pharmaceutically acceptable salt or zwitterionic form thereof, and thereby assists in the delivery of the compound. Suitable agents that may act in this capacity include a monoclonal or polyclonal antibody, a protein or a liposome.

The pharmaceutical composition of other embodiments of the invention may consist of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols of compounds of the invention may be delivered in single phase, bi-phasic, or tri-phasic systems in order to deliver the active ingredient(s). Delivery of the aerosol includes the necessary container, activators, valves, subcontainers, and the like, which together may form a kit. One skilled in the art, without undue experimentation may determine preferred aerosols.

In some embodiments, the pharmaceutical compositions of embodiments the invention may be prepared by methodology well known in the pharmaceutical art. For example, a pharmaceutical composition intended to be administered by injection can be prepared by combining a compound of structure (I), or a pharmaceutically acceptable salt or zwitterionic form thereof, with sterile, distilled water so as to form a solution. A surfactant may be added to facilitate the formation of a homogeneous solution or suspension. Surfactants are compounds that non-covalently interact with the compound of structure (I), or a pharmaceutically acceptable salt or zwitterionic form thereof, so as to facilitate dissolution or homogeneous suspension of the compound of structure (I), or a pharmaceutically acceptable salt or zwitterionic form thereof in the aqueous delivery system.

The methods of the present invention include administering a compound of structure (I) or a pharmaceutically acceptable salt or zwitterionic form thereof, in a therapeutically effective amount, which will vary depending upon a variety of factors including the activity of the specific compound employed; the metabolic stability and length of action of the compound; the age, body weight, general health, sex, and diet of the patient; the mode and time of administration; the rate of excretion; the drug combination; the severity of the particular disorder or condition; and the subject undergoing therapy.

Compounds of structure (I), or pharmaceutically acceptable salts or zwitterionic forms thereof, may also be administered simultaneously with, prior to, or after administration of one or more additional therapeutic agents. Such combination therapy includes administration of a single pharmaceutical dosage formulation which contains a compound of structure (I), or a pharmaceutically acceptable salt or zwitterionic form thereof, and one or more additional active agents, as well as administration of the compound of structure (I), or a pharmaceutically acceptable salt or zwitterionic form thereof and each active agent in its own separate pharmaceutical dosage formulation. For example, a compound of structure (I), or a pharmaceutically acceptable salt or zwitterionic form thereof and the other active agent can be administered to the patient together in a single oral dosage composition such as a tablet or capsule, or each agent administered in separate oral dosage formulations. Where separate dosage formulations are used, the compounds of the invention and one or more additional active agents can be administered at essentially the same time, i.e., concurrently, or at separately staggered times, i.e., sequentially; combination therapy is understood to include all these regimens.

In some embodiments, the concentration of the compound of structure (I), or a pharmaceutically acceptable salt or zwitterionic form thereof provided in the pharmaceutical compositions of the present invention is less than 100%, 90%, 80%, 70%, 60%, 50%, 40%, 30%, 20%, 19%, 18%, 17%, 16%, 15%,14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v or v/v.

In some embodiments, the concentration of the compound of structure (I), or a pharmaceutically acceptable salt or zwitterionic form thereof provided in the pharmaceutical compositions of the present invention is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% w/w, w/v, or v/v.

In some embodiments, the concentration of the compound of structure (I), or a pharmaceutically acceptable salt or zwitterionic form thereof provided in the pharmaceutical compositions of the present invention is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v.

In some embodiments, the concentration of the compound of structure (I), or a pharmaceutically acceptable salt or zwitterionic form thereof provided in the pharmaceutical compositions of the present invention is in the range from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w, w/v or v/v.

In some embodiments, the amount the compound of structure (I), or a pharmaceutically acceptable salt or zwitterionic form thereof provided in the pharmaceutical compositions of the present invention is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of the compound of structure (I), or a pharmaceutically acceptable salt or zwitterionic form thereof provided in the pharmaceutical compositions of the present invention is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

In some embodiments, the amount of the compound of structure (I), or a pharmaceutically acceptable salt or zwitterionic form thereof provided in the pharmaceutical compositions of the present invention is in the range of 0.0001-10 g, 0.0005-9 g, 0.001-8 g, 0.005-7 g, 0.01-6 g, 0.05-5 g, 0.1-4 g, 0.5-4 g, or 1-3 g.

### EXAMPLES

### Example 1

### In Vitro Activity of Alvocidib in Androgen-Resistant Prostate Cancer Cells

The compound of structure (I), and pharmaceutically acceptable salts and zwitterionic forms thereof, is converted in vivo to alvocidib (see U.S. Patent Publication No. US 2016/0340376, the full disclosure of which is hereby incorporated by reference in its entirety). Therefore, the in vitro effects of alvocidib were evaluated in multiple prostate cancer cell lines with varied sensitivity to androgen. PC3 is an AR-negative prostate cancer cell line with little to no expression of PSA and low sensitivity to androgens. VCAP is a prostate cancer cell line that is positive for ARv7, and can grow in an androgen-independent manner. UNCAP is an androgen-dependent prostate cancer cell line. 22Rv1 is a prostate cancer cell line that is positive for ARv7, has low sensitivity to androgen, and is derived from a xenograft that was serially propagated in mice after castration-induced regression and relapse of the parental, androgen-dependent CWR22 xenograft.

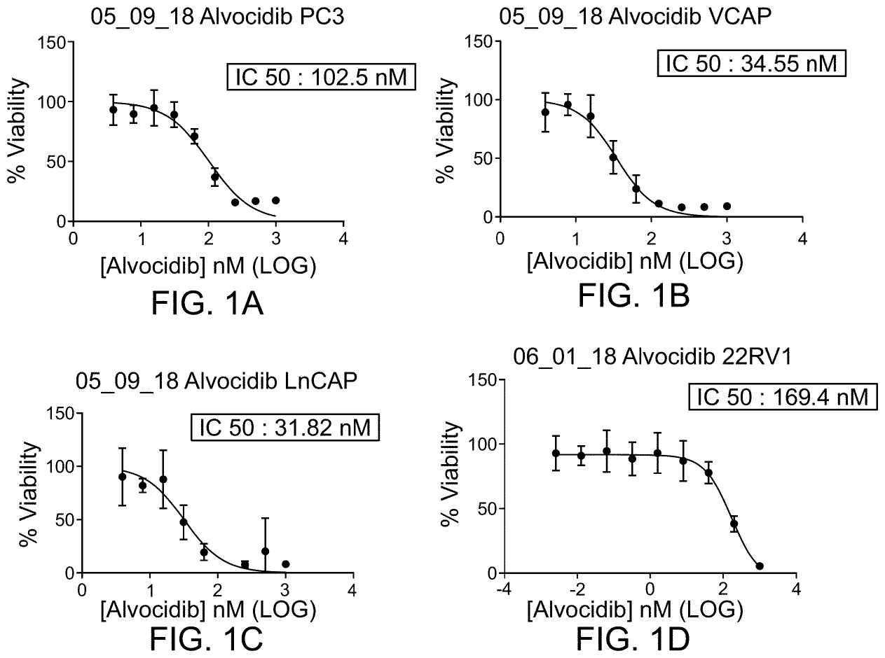

Prostate cancer cell lines PC3, VCAP, LNCaP, and 22Rv1 were treated with a range of alvocidib doses between 3.9 nM-1000 nM for PC3, VCAP, and LNCaP and 0.0026 nM-1000 nM for 22Rv1, and cell viability was measured. FIG. 1A shows the viability of PC3 cells following alvocidib treatment, and shows an IC50 of 102.5 nM. FIG. 1B shows viability of VCAP cells following alvocidib treatment, and an IC50 of 34.55 nM. FIG. 1C shows viability of LNCaP cells following alvocidib treatment, and shows an IC50 of 31.82 nM. FIG. 1D shows viability of 22RV1 cells following alvocidib treatment, and shows an IC50 of 169.4 nM. Cell viability may be assessed, for example, using CellTiter-Glo according to manufacturer protocol.

### Example 2

### In Vitro Effects of Alvocidib on Serum Stimulated Prostate Cancer Cells

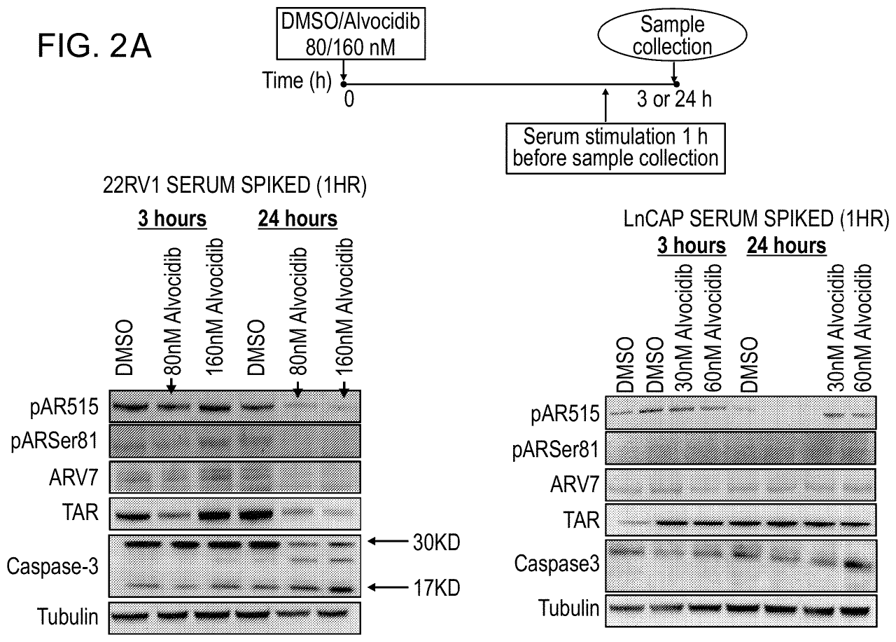

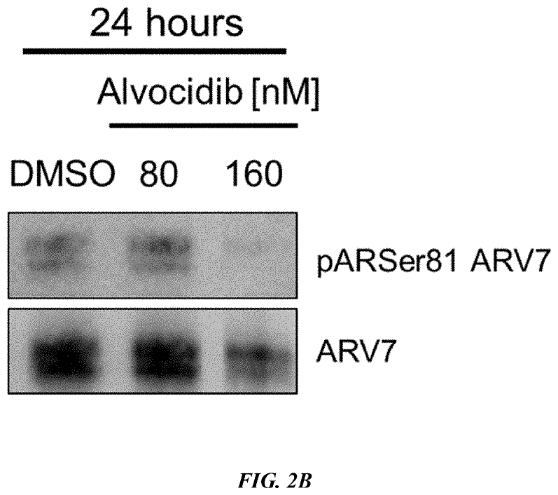

In a first experiment to evaluate the effects of alvocidib on serum stimulated prostate cancer cells, androgen receptor expression was assessed by immunoblotting. FIG. 2A, top panel, shows a diagram of the experimental protocol. The prostate cancer cells, 22Rv1 or LNCaP, were treated with DMSO or alvocidib (80 nM or 160 nM), for either 3 hours or 24 hours, and were serum stimulated one hour prior to sample collection (or the cells were serum starved as a control). FIG. 2A, bottom left panel, shows the effects of alvocidib treatment (3-hour or 24-hour treatment) on protein levels of: pAR515; pARSer81; ARv7; total AR (TAR); caspase-3; and tubulin (as loading control) in serum-stimulated 22Rv1 cells. FIG. 2A, right panel, shows the effects of alvocidib treatment (3-hour or 24-hour treatment) on protein levels of: pAR515; pARSer81; ARv7; total AR (TAR); caspase-3; and tubulin (as loading control) in serum-stimulated LNCaP cells. FIG. 2B shows the effects of alvocidib treatment (24-hour treatment) on protein levels of pARSer81 ARV7 and ARV7. As can be seen in FIGS. 2A and 2B, alvocidib lowers phosphor and total-AR levels after 24 hours of treatment.

In another experiment, androgen receptor expression and functionality were assessed using quantitative real-time PCR measurement of the mRNA level of transmembrane protease, serine 2 (TMPRSS2) mRNA levels in prostate cancer cell line 22Rv1, as described in Chen et al., 2012. JBC. 287:8571. TMPRSS1 is an androgen-responsive gene, which is transcriptionally regulated by androgen receptor. An androgen response may be driven by the addition of exogenous testosterone, or by serum stimulation (which contains androgens), in prostate cancer cell lines.

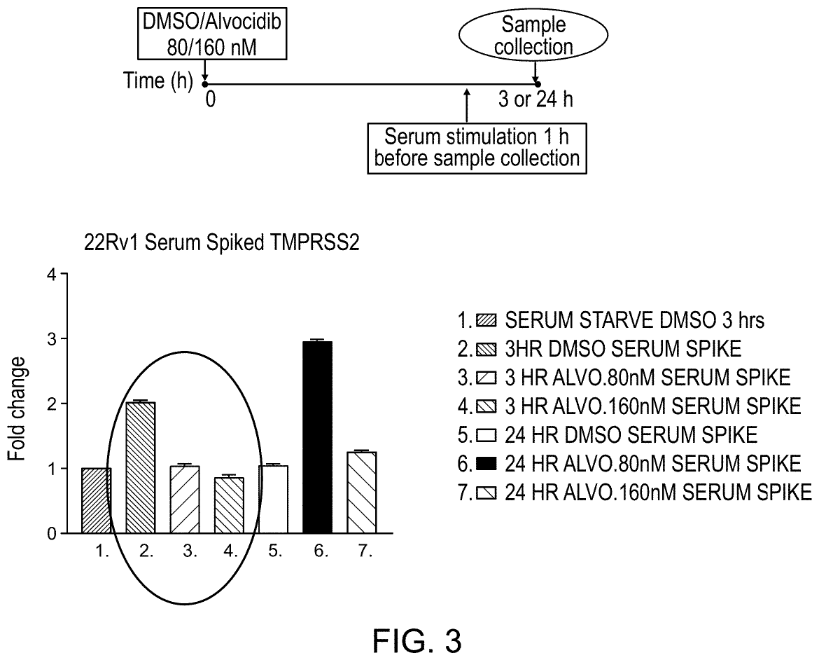

FIG. 3 shows the effects of alvocidib treatment (3 hour or 24 hour treatment) on TMPRSS2 expression in serum-stimulated 22Rv1 cells. FIG. 3, top panel, shows a flowchart of the experimental protocol. The 22Rv1 cells were treated with DMSO or alvocidib (80 nM or 160 nM) for either 3 hours or 24 hours, and serum stimulated one hour prior to sample collection (or the cells were serum starved as a control). FIG. 3, bottom panel, shows the fold change in TMPRSS2 expression for each condition. As can be seen in the circle, alvocidib inhibited serum-stimulated induction of TMPRSS2.

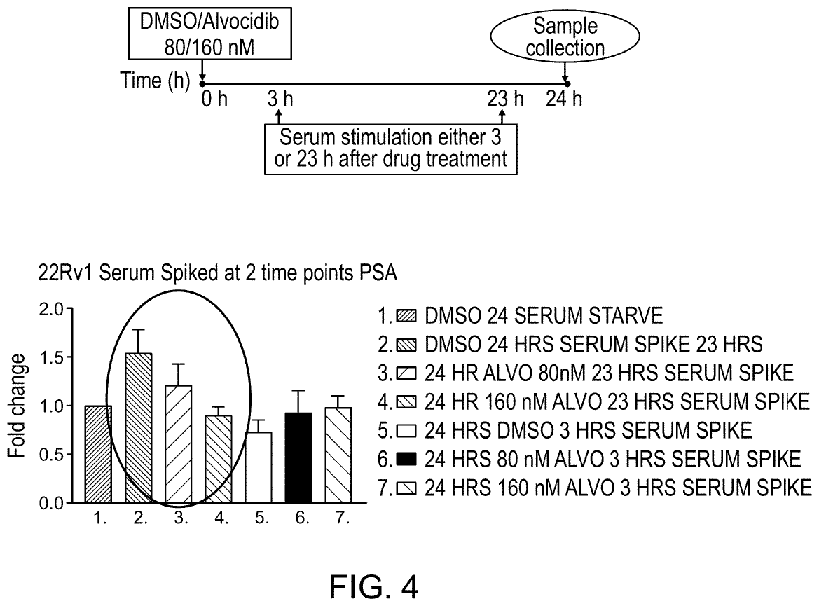

FIG. 4 shows the effects of alvocidib treatment (24-hour treatment) on PSA expression in serum-stimulated 22Rv1 cells, stimulated with serum 3 hours or 23 hours following alvocidib treatment. FIG. 4, top panel, shows a flowchart of the experimental protocol. The 22Rv1 cells were treated with DMSO or alvocidib (80 nM or 160 nM) for 24 hours, and serum stimulated either 3 hours or 23 hours following treatment (or the cells were serum starved as a control). FIG. 4, bottom panel, shows the fold change in PSA expression for each condition. As can be seen in the circle, alvocidib inhibited serum stimulated induction of PSA.

### Example 3

### Efficacy Study in the 22RV1 Xenograft Prostate Cancer Model

The objective of this study was to evaluate the in vivo therapeutic efficacy of the compound of structure (I) or a pharmaceutically acceptable salt or zwitterionic form thereof in the treatment of a subcutaneous 22Rv1 human prostate cancer xenograft model. For the 22Rv1 model, efficacy of the compound of structure (I) was evaluated in male BALM nude mice. Cobalt-60 irradiation of all mice was performed 2 days before the tumor inoculation at 2 Gy (1Gy=100 rads). Each mouse was inoculated at the right flank with 1×10⁷ tumor cells in 0.1 ml of PBS mixed with matrigel (1:1). The date of tumor cell inoculation was denoted as day 0.

Castration was performed when mean tumor volume reached approximately 200 mm³. Mice were anesthetized with ketamine/xylazine; surgical castration was performed via a midline scrotal incision allowing bilateral access to the hemiscrotal contents; after exposing each testicle, a 6-0 Vicryl suture was used to ligate the spermatic cord and then remove the testicle; the scrota and skin was then closed with 6-0 Vicryl suture, separately. Treatments started when the mean tumor volume re-grew to approximately 100 mm³.

After tumors reached the appropriate size, approximately 100-200 mm³, mice were randomized into treatment groups 1-11, which are shown in Table 1. Each treatment was administered starting on day 15, at a dosing volume of 5 μL/g and continued for 21 days (or 22 days for groups with treatment at Q7D). For groups with treatment at Q7D×3 weeks, dosing was performed on days 1, 8, and 15 post-randomization, and the study was terminated on day 22 post randomization. The combined treatment intervals were each 0 h, and the interval for BID was 8h. Randomization was performed based on the “Matched distribution” method (StudyDirector™ software, version 3.1.399.19) randomized block design.

TABLE 1
Treatment groups for the 22Rv1 xenograft study.

| | | | | | | Dosing |
|---|---|---|---|---|---|---|
| | | | | Dose level | | Frequency & |
| Group | Castrated | No. | Treatment | (mg/kg) | ROA | Duration |
| 1 | NO | 6 | Vehicle 1 | 0 | p.o. | QD × 21 days |
| | | | Vehicle 2 | 0 | p.o. | QD × 21 days |
| 2 | Yes | 8 | Vehicle 1 | 0 | p.o. | QD × 21 days |
| | | | Vehicle 2 | 0 | p.o. | QD × 21 days |
| 3 | Yes | 8 | Vehicle 1 | 0 | p.o. | QD × 21 days |
| | | | Enzalutamide | 30 | p.o. | QD × 21 days |
| 4 | Yes | 8 | Vehicle 1 | 0 | p.o. | QD × 21 days |
| | | | Abiraterone | 50 | i.p. | QD × 21 days |
| 5 | Yes | 8 | Vehicle 2 | 0 | p.o. | QD × 21 days |
| | | | Docetaxel | 10 | i.p. | Q7D × 21 days |
| 6 | Yes | 8 | Vehicle 2 | 0 | p.o. | QD × 21 days |
| | | | Cmpd Str (I) | 1.25 | p.o. | BID × 21 days |
| 7 | Yes | 8 | Vehicle 2 | 0 | p.o. | QD × 21 days |
| | | | Cmpd Str (I) | 10 | p.o. | Q7D × 21 days |
| 8 | Yes | 8 | Cmpd Str (I) | 1.25 | p.o. | BID × 21 days |
| | | | Enzalutamide | 30 | p.o. | QD × 21 days |
| 9 | Yes | 8 | Cmpd Str (I) | 1.25 | p.o. | BID × 21 days |
| | | | Docetaxel | 10 | i.p. | Q7D × 21 days |
| 10 | Yes | 8 | Vehicle 1 | 0 | p.o. | QD × 21 days |
| | | | ABT199 | 100 | p.o. | QD × 21 days |
| 11 | Yes | 8 | Cmpd Str (I) | 1.25 | p.o. | BID × 21 days |
| | | | ABT199 | 100 | p.o. | QD × 21 days |

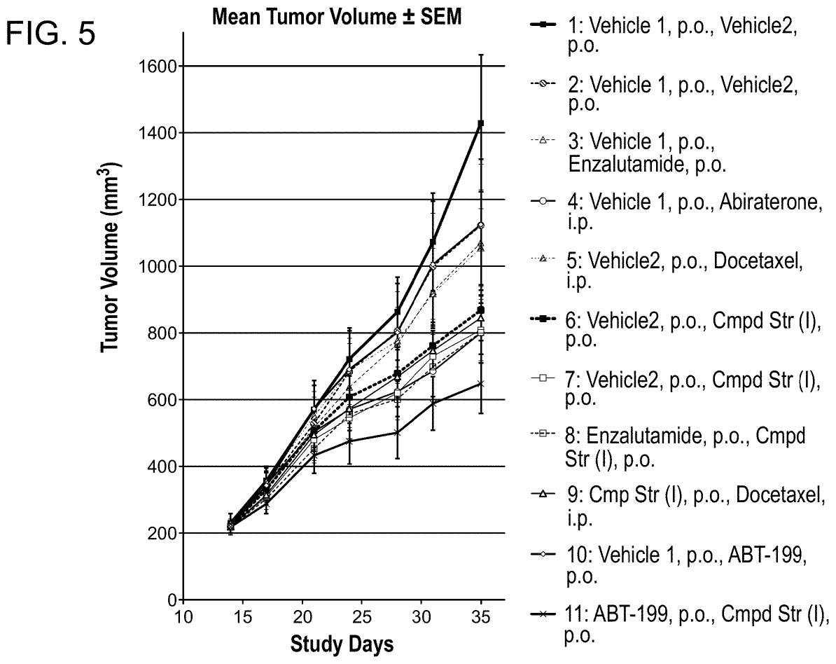

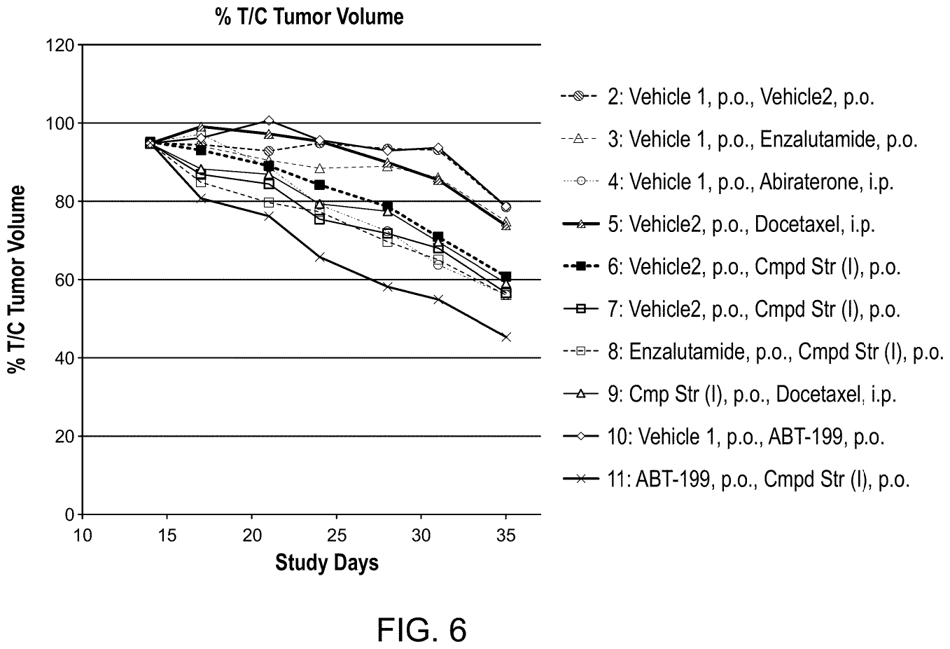

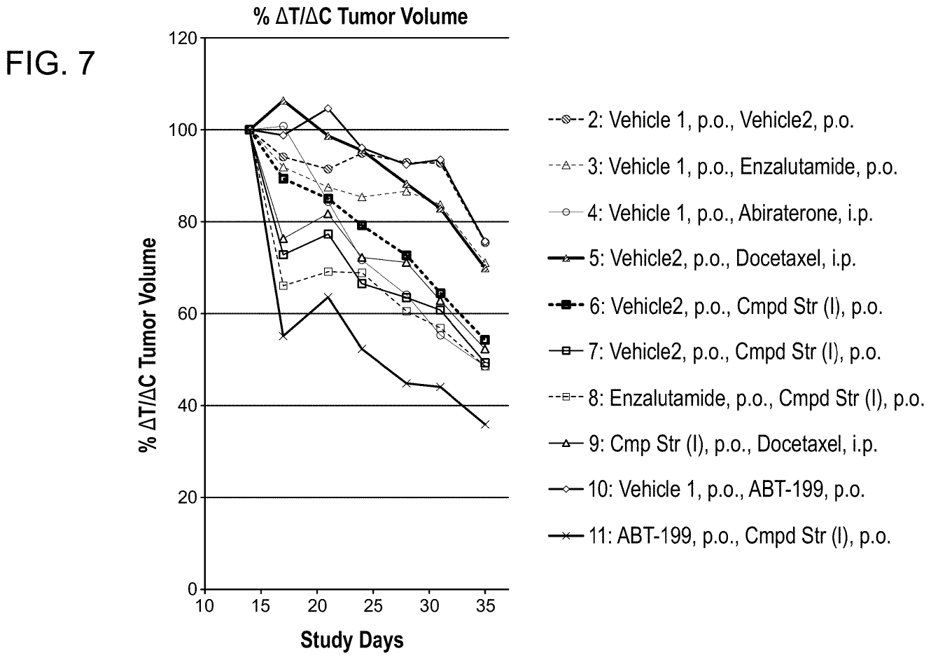

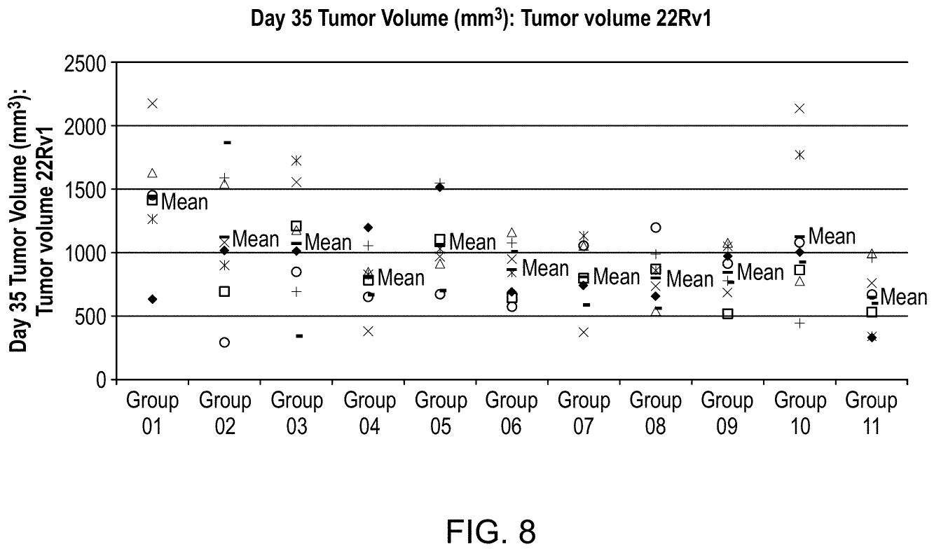

Tumor volumes were measured twice per week after randomization in two dimensions using a caliper, and the volume was expressed in mm³ using the formula: “V=(L×W×W)/2,” where V is tumor volume, L is tumor length (the longest tumor dimension) and W is tumor width (the longest tumor dimension perpendicular to L). The average tumor volume for each group is shown in FIG. 5. The average tumor volume for each group, as a percentage of the average tumor volume of the control group (group 1), is shown in FIG. 6. The average percentage change in tumor volume for each group, as a ratio of the average percentage change in tumor volume of the control group is shown in FIG. 7. The percentage shown in FIG. 7 (% T/C) is calculated as mean(T)/mean(C)*100%, with “T” representing tumor volume and “C” representing tumor volume for group 1. The tumor volume for individuals of each group at day 35 of the study is shown in FIG. 8.

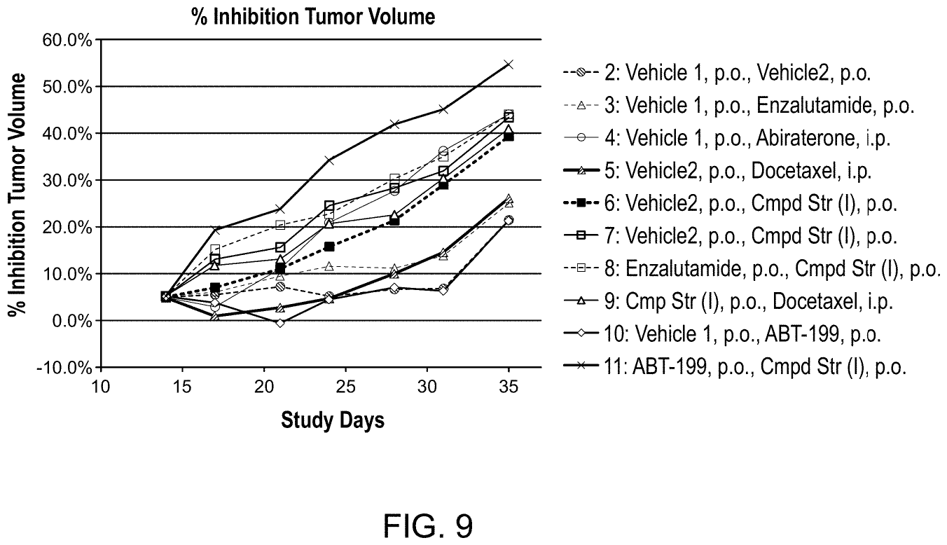

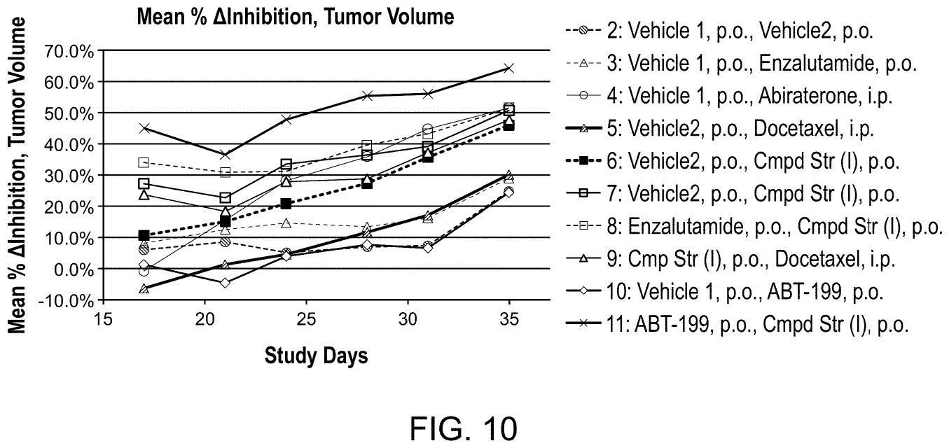

Tumor growth inhibition (TGI) or “Inhibition”: TGI % is an indication of anti-cancer activity, and expressed as: Mean % Inhibition=(mean(C)−mean(T))/mean(C)*100%. T and C are the mean tumor volume (or weight) of the treated groups and control group (group 1), respectively, on a given day. The average percentage inhibition of tumor growth for each group, as compared to the control group, is shown in FIG. 9. The average percent change in inhibition of tumor growth for each group, as compared to the control group, is shown in FIG. 10. The percentages shown in FIG. 10 are calculated as % AT/C=(mean(T)−mean(TO))/(mean(C)−mean(C0))*100%, with “0” indicating the initial timepoint.

As can be seen in FIGS. 5-10, castration resulted in a small decrease in tumor growth that was similar to the following treatments: venetoclax, enzalutamide, and docetaxel, indicating that these drugs as monotherapies were not active in the 22Rv1 xenograft model. Additionally, the compound of structure (I) as a monotherapy at two different doses and schedules showed modest activity. However, the compound of structure (I) in combination with ABT199 (venetoclax) showed robust anti-cancer activity in the 22Rv1 model, demonstrating 64% tumor growth inhibition in the 22Rv1 model.

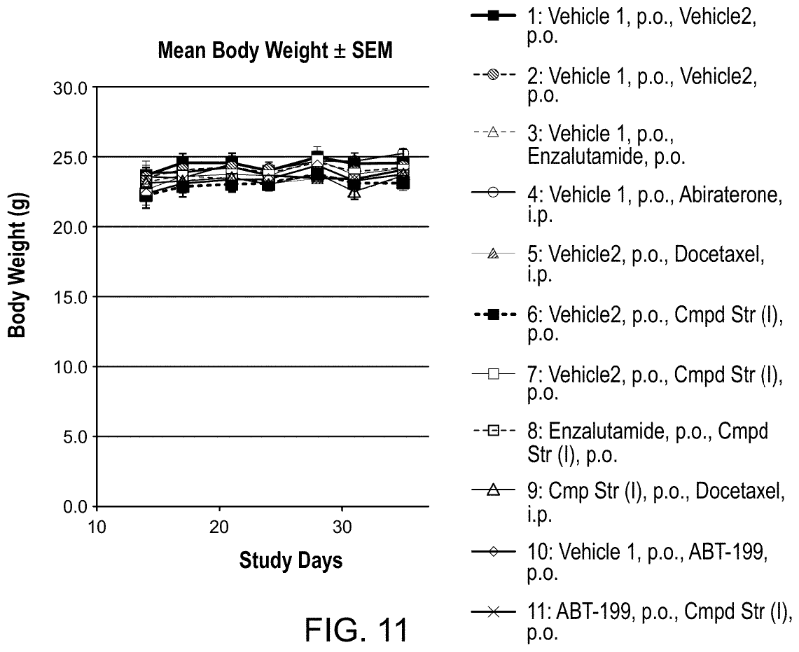

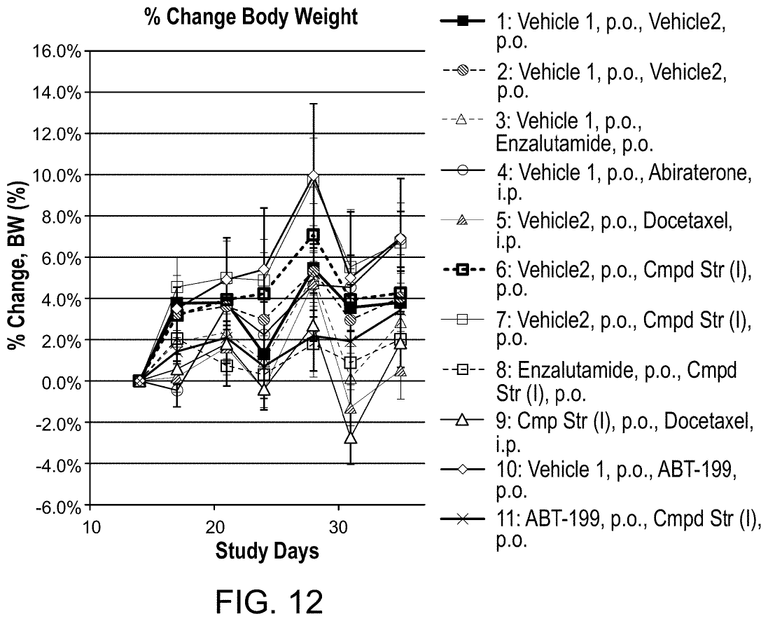

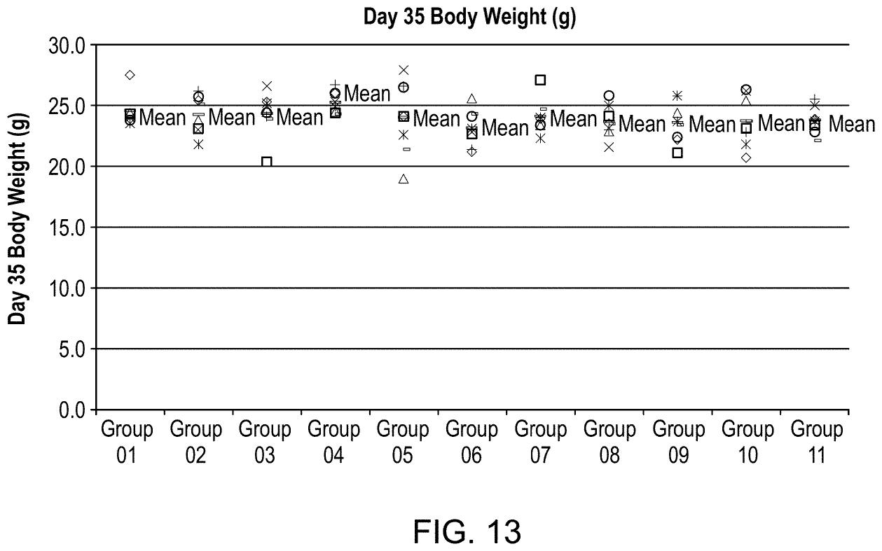

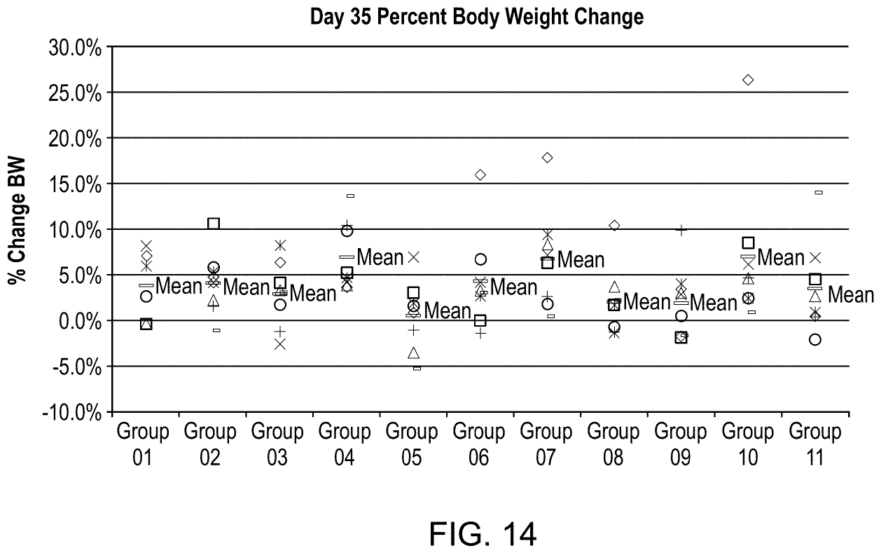

Throughout the study, body weight was also monitored. The average body weight for each group is shown in FIG. 11. The average percentage body weight change for each group is shown in FIG. 12. The body weight for the individuals of groups 1-11 (as defined in Table 1) on day 35 is shown in FIG. 13. The percentage body weight change at day 35 for individuals of groups 1-11 is shown in FIG. 14.

Following the 21-day treatment period, the mice were observed for 7 days post final dose, or until individual tumor volume of group 1 reached 3000 mm³, or the mean tumor volume of group 1 reached 2000 mm³. Tumor weight was measured at the end of study, and tumors were harvested (24 hours post final dose for QD treatment groups; 12 hours post final dose for BID treatment groups and 6 days post final dose for Q7D treatment groups): for each tumor, ½ was utilized for snap freezing, and ½ was utilized for FFPE.

Tissue samples acquired from the study (Groups 1, 5, 6, 9, 10, 11) were homogenized by bead homogenizer (20 s at 4.5 m/s) (Fisherbrand Bead Mill 24 Homogenizer, Fisher Scientific) and lysed in 1% Triton buffer (Cell Signaling Technology, Cat #9803) with protease and phosphatase inhibitor (Thermo Fisher Scientific, Cat #1861281, Lot #TA261245). Individual samples were cleared by centrifugation at 15,000 RPM, quantified and pooled for each treatment group, and protein expression was analyzed by Western blot. 30 ng of protein was loaded into a 4-12% gel and transferred to a PVDF membrane. Blocking, anti-cleaved caspase 3 (Cell Signaling Technology, Cat #9664, Lot #21; 1:1,000), anti-β-actin (Proteintech, Cat #HRP-60008; 1:10,000), and secondary staining was done in 5% milk. Anti-cMyc (Cell Signaling Technology, Cat #4572; 1:1,000), and anti-MCL-1 (Cell Signaling Technology, Cat #4572; 1:1,000) antibodies were diluted in BSA.

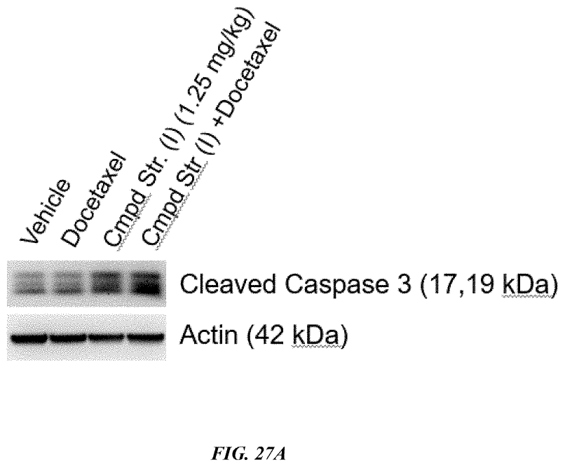

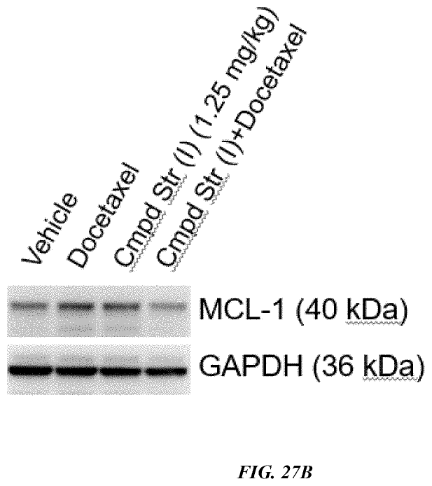

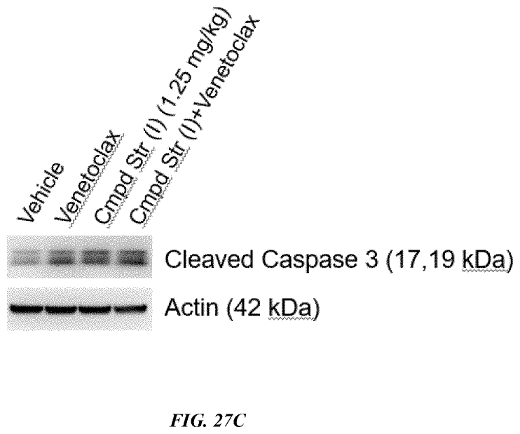

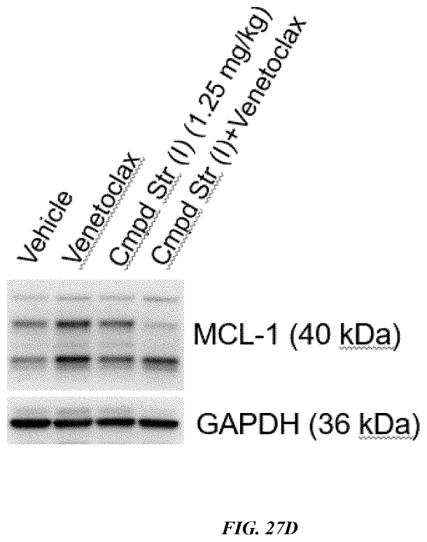

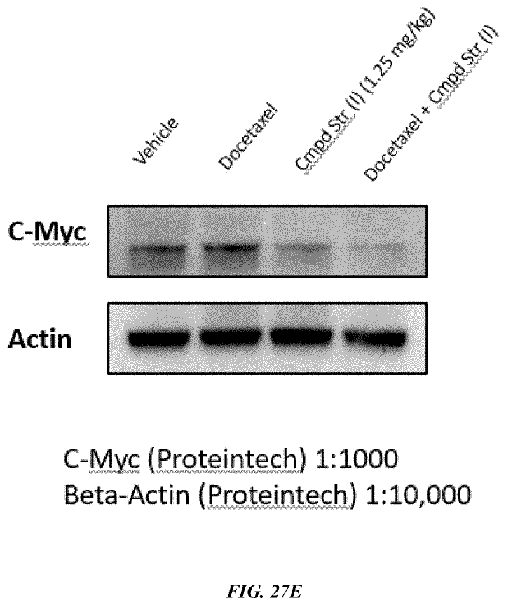

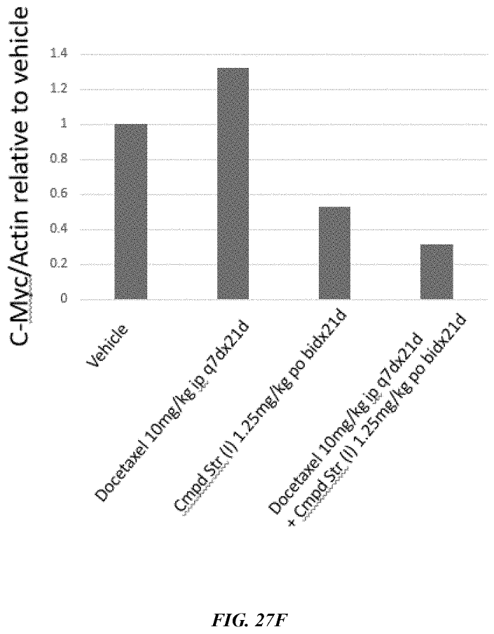

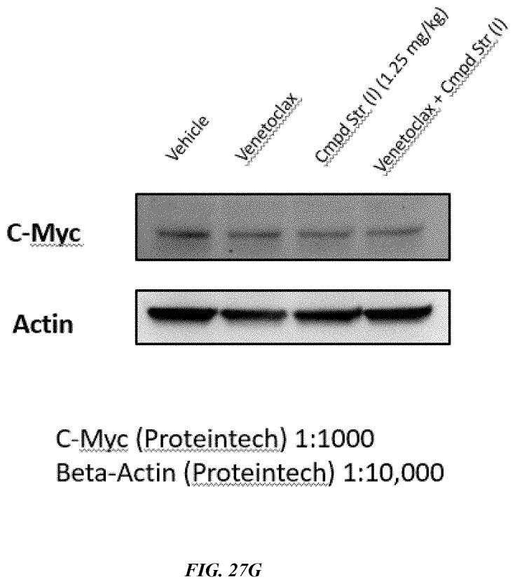

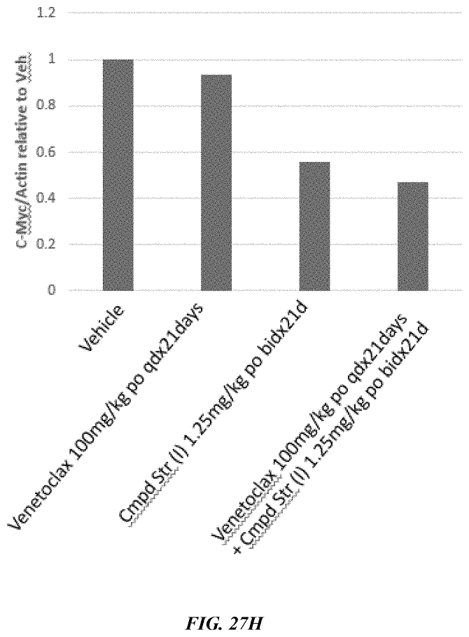

FIGS. 27A and C are images of Western blots, and show the amount of cleaved caspase 3 as a function of treatment group. FIGS. 27B and D are images of Western blots, and show the amount of MCL-1 as a function of treatment group. FIGS. 27E and G are images of Western blots, and show the amount of C-Myc as a function of treatment group. FIGS. 27F and H are bar graphs, and show the ratios of C-Myc/actin relative to vehicle in the various treatment groups depicted in the Western blots of FIGS. 27E and G, respectively.

Combined treatment with Cmpd Str (I) and venetoclax induced cleaved caspase 3 compared to vehicle, as well as single treatment groups. Thus, the tumor growth inhibition resulting from combined treatment of Cmpd Str (I) and venetoclax corresponded with increased cleavage of caspase 3 protein by Western blotting of tumor lysates.

### Example 4

### Efficacy Study in the C4-2 Xenograft Prostate Cancer Model

The objective of this study is to evaluate the in vivo therapeutic efficacy of the compound of structure (I) in the treatment of a subcutaneous C4-2 human prostate cancer xenograft model. C4-2 cells are an androgen-independent prostate cancer cell line established from the androgen-dependent prostate cancer cell line LNCaP. For the C4-2 model, efficacy is evaluated in male NCG (NOD-Prkdc^{em26Cd52}Il2rg^{em26Cd22}Nju) mice. Cobalt-60 irradiation of all mice is performed 2 days before the tumor inoculation at 2 Gy (1Gy=100 rads). Each mouse is inoculated at the right flank with 5×10⁶ tumor cells (C4-2 cells) in 0.1 ml of PBS mixed with matrigel (1:1). The date of tumor cell inoculation is denoted as day 0.

Castration is performed when mean tumor volume reaches approximately 200 mm³. Mice are anesthetized with ketamine/xylazine; surgical castration is performed via a midline scrotal incision allowing bilateral access to the hemiscrotal contents; after exposing each testicle, a 6-0 Vicryl suture is used to ligate the spermatic cord and then remove the testicle; the scrota and skin are then closed with 6-0 Vicryl suture, separately. After castration, the tumors may shrink (tumor regression), so the randomization and treatments start when the mean tumor volume re-grows to approximately 100 mm³. If the mean tumor volume is very small due to tumor regression, the randomization is performed based on body weight.

After tumors reach the appropriate size, as noted above, mice are randomized into treatment groups 1-9, which are shown in Table 2. Each treatment is administered at a dosing volume of 5 μL/g and continues for 21 days (or 22 days for groups with treatment at Q7D). For groups with treatment at Q7D×3 weeks, dosing is performed on days 1, 8, and 15 post-randomization, and the study is terminated on day 22 post randomization. The combined treatment intervals are each 0 h, and the interval for BID is 8h. Randomization is performed based on “Matched distribution” method (StudyDirector™ software, version 3.1.399.19) randomized block design.

TABLE 2
Treatment groups for the C4-2 xenograft study.

| | | | | Dose | | Dosing |
|---|---|---|---|---|---|---|
| | | | | level | | Frequency & |
| Group | Castrated | No. | Treatment | (mg/kg) | ROA | Duration |
| 1 | NO | 6 | Vehicle 1 | 0 | p.o. | QD × 21 days |
| | | | Vehicle 2 | 0 | p.o. | QD × 21 days |
| 2 | Yes | 8 | Vehicle 1 | 0 | p.o. | QD × 21 days |
| | | | Vehicle 2 | 0 | p.o. | QD × 21 days |
| 3 | Yes | 8 | Vehicle 1 | 0 | p.o. | QD × 21 days |
| | | | Enzalutamide | 30 | p.o. | QD × 21 days |
| 4 | Yes | 8 | Vehicle 1 | 0 | p.o. | QD × 21 days |
| | | | Abiraterone | 50 | i.p. | QD × 21 days |
| 5 | Yes | 8 | Vehicle 2 | 0 | p.o. | QD × 21 days |
| | | | Docetaxel | 2.5 | i.p. | Q7D × 3 weeks |
| 6 | Yes | 8 | Vehicle 2 | 0 | p.o. | QD × 21 days |
| | | | Cmpd Str (I) | 1.25 | p.o. | BID × 21 days |
| 7 | Yes | 8 | Vehicle 2 | 0 | p.o. | QD × 21 days |
| | | | Cmpd Str (I) | 10 | p.o. | Q7D × 3 weeks |
| 8 | Yes | 8 | Cmpd Str (I) | 1.25 | p.o. | BID × 21 days |
| | | | Enzalutamide | 30 | p.o. | QD × 21 days |
| 9 | Yes | 8 | Cmpd Str (I) | 1.25 | p.o. | BID × 21 days |
| | | | Docetaxel | TBD | i.p. | Q7D × 3 weeks |

Following the 21-day treatment period, the mice are observed for 7 days post final dose, or until individual tumor volume of group 1 reaches 3000 mm³, or the mean tumor volume of group 1 reaches 2000 mm³.

Tumor volumes are measured twice per week after randomization in two dimensions using a caliper, and the volume is expressed in mm³ using the formula: “V=(L×W×W)/2,” where V is tumor volume, L is tumor length (the longest tumor dimension) and W is tumor width (the longest tumor dimension perpendicular to L). Tumor weight is measured at the end of study. Tumor growth inhibition (TGI): TGI % is an indication of anti-cancer activity, and expressed as: TGI (%)=100×(1−T/C). T and C are the mean tumor volume (or weight) of the treated and control groups, respectively, on a given day. Tumors are harvested at the end of study (24 hrs post final dose for QD treatment groups; 12 hrs post final dose for BID treatment groups and 6 days post final dose for Q7D treatment groups): for each tumor, ½ is utilized for snap freezing, and ½ is utilized for FFPE.

### Example 5

### Efficacy Study in the LNCAP FGC Xenograft Prostate Cancer Model

The objective of this study is to evaluate the in vivo therapeutic efficacy of the compound of structure (I) in the treatment of a subcutaneous LNCaP FGC human prostate cancer xenograft model. LNCaP clone FGC is an androgen-dependent prostate cancer cell line established from prostate cancer cell line LNCaP. For the LNCaP clone FGC model, efficacy is evaluated in male NCG (NOD-Prkdc^{em26Cd52}Il2rg^{em26Cd22}Nju) mice. Each mouse is implanted subcutaneously with an androgen pellet (1.5 mg/pellet, with testosterone propionate powder) at the left flank 1 day before the tumor inoculation. Cobalt-60 irradiation of all mice is performed 2 days before the tumor inoculation at 2 Gy (1 Gy=100 rads). Each mouse is inoculated at the right flank with 5×10⁶ tumor cells (LNCaP clone FGC cells) in 0.1 ml of PBS mixed with matrigel. (1:1). The date of tumor cell inoculation is denoted as day 0.

Castration is performed when mean tumor volume reaches approximately 200 mm³. Mice are anesthetized with ketamine/xylazine; surgical castration is performed via a midline scrotal incision allowing bilateral access to the hemiscrotal contents; after exposing each testicle, a 6-0 Vicryl suture is used to ligate the spermatic cord and then remove the testicle; the scrota and skin are then closed with 6-0 Vicryl suture, separately. After castration, the tumors may shrink (tumor regression), so the randomization and treatments start when the mean tumor volume re-grows to approximately 200 mm³. If the mean tumor volume is very small due to tumor regression, the randomization is performed based on body weight.

After tumors reach the appropriate size as noted above, mice are randomized into treatment groups 1-9, which are shown in Table 3. Each treatment is administered at a dosing volume of 5 μL/g and will continue for 21 days (or 22 days for groups with treatment at Q7D). For groups with treatment at Q7D×3 weeks, dosing is performed on days 1, 8, and 15 post-randomization, and the study is terminated on day 22 post randomization. The combined treatment intervals are each 0 h, and the interval for BID is 8h. Randomization is performed based on “Matched distribution” method (StudyDirector™ software, version 3.1.399.19)/randomized block design.

TABLE 3
Treatment groups for the LNCaP FGC xenograft study.

| | | | | Dose | | Dosing |
|---|---|---|---|---|---|---|
| | Cas- | | | level | | Frequency & |
| Group | trated | No. | Treatment | (mg/kg) | ROA | Duration |
| 1 | NO | 6 | Vehicle 1 | 0 | p.o. | QD × 21 days |
| | | | Vehicle 2 | 0 | p.o. | QD × 21 days |
| 2 | Yes | 8 | Vehicle 1 | 0 | p.o. | QD × 21 days |
| | | | Vehicle 2 | 0 | p.o. | QD × 21 days |
| 3 | Yes | 8 | Vehicle 1 | 0 | p.o. | QD × 21 days |
| | | | Enzalutamide | 30 | p.o. | QD × 21 days |
| 4 | Yes | 8 | Vehicle 1 | 0 | p.o. | QD × 21 days |
| | | | Abiraterone | 50 | i.p. | QD × 21 days |
| 5 | Yes | 8 | Vehicle 2 | 0 | p.o. | QD × 21 days |
| | | | Docetaxel | TBD | i.p. | Q7D × 3 weeks |
| 6 | Yes | 8 | Vehicle 2 | 0 | p.o. | QD × 21 days |
| | | | Cmpd Str (I) | 1.25 | p.o. | BID × 21 days |
| 7 | Yes | 8 | Vehicle 2 | 0 | p.o. | QD × 21 days |
| | | | Cmpd Str (I) | 10 | p.o. | Q7D × 3 weeks |
| 8 | Yes | 8 | Cmpd Str (I) | 1.25 | p.o. | BID × 21 days |
| | | | Enzalutamide | 30 | p.o. | QD × 21 days |
| 9 | Yes | 8 | Cmpd Str (I) | 1.25 | p.o. | BID × 21 days |
| | | | Docetaxel | TBD | i.p. | Q7D × 3 weeks |

Following the 21-day treatment period, the mice are observed for 7 days post final dose, or until individual tumor volume of group 1 reaches 3000 mm³, or the mean tumor volume of group 1 reaches 2000 mm³.

Tumor volumes are measured twice per week after randomization in two dimensions using a caliper, and the volume is expressed in mm³ using the formula: “V=(L×W×W)/2,” where V is tumor volume, L is tumor length (the longest tumor dimension) and W is tumor width (the longest tumor dimension perpendicular to L). Tumor weight is measured at the end of study. Tumor growth inhibition (TGI): TGI % is an indication of anti-cancer activity, and expressed as: TGI (%)=100×(1−T/C). T and C are the mean tumor volume (or weight) of the treated and control groups, respectively, on a given day. Tumors are harvested at the end of study (24 hrs post final dose for QD treatment groups; 12 hrs post final dose for BID treatment groups and 6 days post final dose for Q7D treatment groups): for each tumor, ½ is utilized for snap freezing, and ½ is utilized for FFPE.

### Example 6

### Results of the Efficacy Study in the C4-2 Xenograft Prostate Cancer Model

The objective of this study was to evaluate the in vivo therapeutic efficacy of the compound of structure (I) in the treatment of a subcutaneous C4-2 human prostate cancer xenograft model. C4-2 cells are an androgen-independent prostate cancer cell line established from the androgen-dependent prostate cancer cell line LNCaP. For the C4-2 model, efficacy was evaluated in male NCG (NOD-) Prkdc^{em26Cd52} Il2rg^{em26Cd22}Nju) mice. Each mouse was inoculated subcutaneously at the right flank region with 5×10⁶ tumor cells (C4-2 cells) in 0.1 ml of PBS mixed with matrigel (1:1). The date of tumor cell inoculation was denoted as day 0.

Castration was performed when mean tumor volume reached approximately 200 mm³. Mice were anesthetized with ketamine/xylazine; surgical castration was performed via a midline scrotal incision allowing bilateral access to the hemiscrotal contents; after exposing each testicle, a 6-0 Vicryl suture was used to ligate the spermatic cord and then remove the testicle; the scrota and skin were then closed with 6-0 Vicryl suture, separately.

The uncastrated mice were randomized when mean tumor volume reached approximately 100-200 mm³. After castration, the randomization was started when the mean tumor volume of castrated mice reached approximately 100-200 mm³. Randomization was performed based on “Matched distribution” method (StudyDirector™ software, version 3.1.399.19).

Mice were enrolled in the study and randomly allocated to study groups as show in Table 4.

TABLE 4
Treatment groups for the C4-2 xenograft study.

| | | | | Dose | | Dosing |
|---|---|---|---|---|---|---|
| | | | | level | | Frequency & |
| Group | Castrated | No. | Treatment | (mg/kg) | ROA | Duration |
| 1 | NO | 6 | Vehicle 1 | 0 | p.o. | QD × 21 days |
| | | | Vehicle 2 | 0 | p.o. | QD × 21 days |
| 2 | Yes | 10 | Vehicle 1 | 0 | p.o. | QD × 21 days |
| | | | Vehicle 2 | 0 | p.o. | QD × 21 days |
| 3 | Yes | 10 | Vehicle 1 | 0 | p.o. | QD × 21 days |
| | | | Enzalutamide | 30 | p.o. | QD × 21 days |
| 4 | Yes | 10 | Vehicle 1 | 0 | p.o. | QD × 21 days |
| | | | Abiraterone | 50 | i.p. | QD × 21 days |
| 5 | Yes | 10 | Vehicle 2 | 0 | p.o. | QD × 21 days |
| | | | Docetaxel | 10 | i.p. | Q7D × 3 weeks |
| 6 | Yes | 10 | Vehicle 2 | 0 | p.o. | QD × 21 days |
| | | | Cmpd Str (I) | 1.25 | p.o. | BID × 21 days |
| 7 | Yes | 10 | Vehicle 2 | 0 | p.o. | QD × 21 days |
| | | | Cmpd Str (I) | 10 | p.o. | Q7D × 3 weeks |
| 8 | Yes | 10 | Cmpd Str (I) | 1.25 | p.o. | BID × 21 days |
| | | | Enzalutamide | 30 | p.o. | QD × 21 days |
| 9 | Yes | 10 | Cmpd Str (I) | 1.25 | p.o. | BID × 21 days |
| | | | Docetaxel | 10 | i.p. | Q7D × 3 weeks |

Each treatment was administered at a dosing volume of 5 μL/g, beginning one day post-grouping (day 1), and was continued for 21 days (or 22 days for groups with treatment at Q7D). For groups with treatment at Q7D×3 weeks, dosing was performed on days 1, 8, and 15 post-randomization, and the study was terminated on day 22 post-randomization. The combined treatment intervals were each 0 h, and the interval for BID was 8h. The study was terminated 38 days post-inoculation for efficacy.

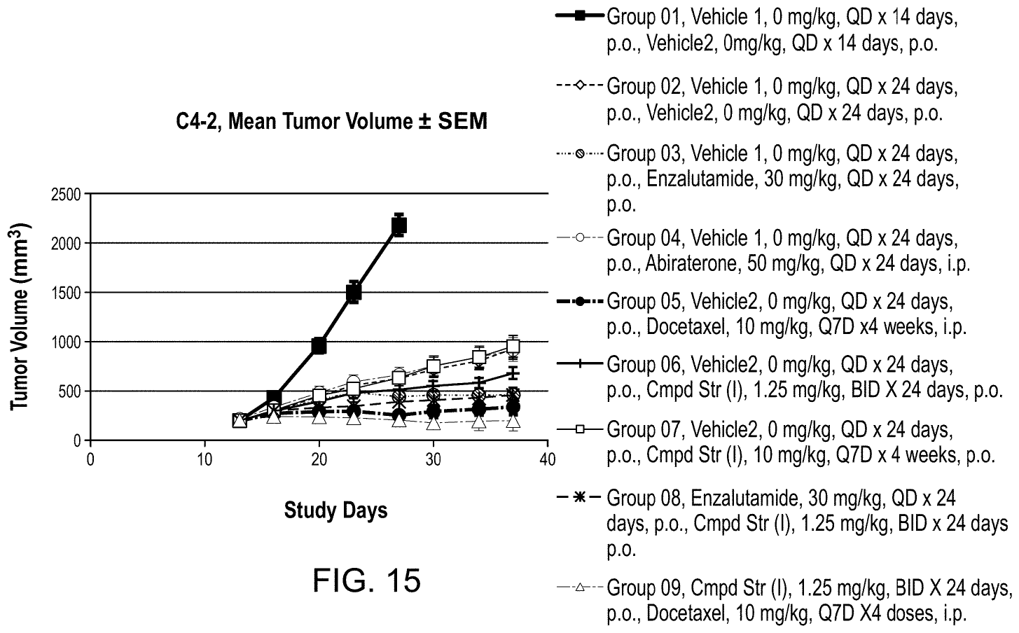

Tumor volumes were measured twice per week after randomization in two dimensions using a caliper, and the volume was expressed in mm³ using the formula: “V=(L×W×W)/2,” where V is tumor volume, L is tumor length (the longest tumor dimension) and W is tumor width (the longest tumor dimension perpendicular to L). FIG. 15 shows the average tumor volume for each group throughout the C4-2 xenograft study.

Tumors were harvested at the end of study (24 hours post final dose for QD treatment groups; 12 hours post final dose for BID treatment groups and 6 days post final dose for Q7D treatment groups): for each tumor, ½ was utilized for snap freezing, and ½ was utilized for FFPE.

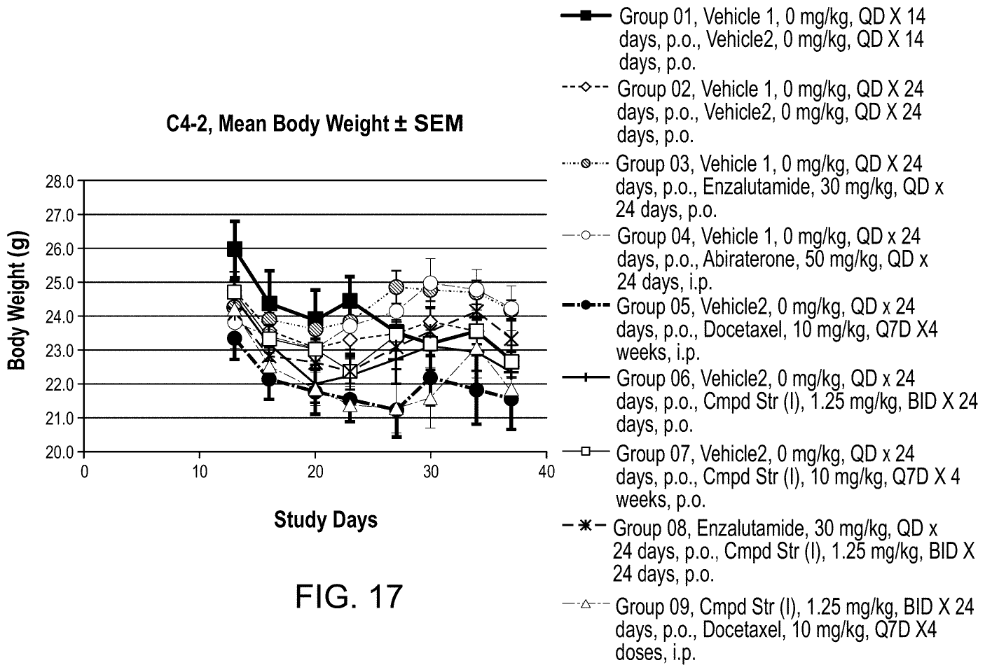

The body weight of all animals was monitored throughout the study. FIG. 17 shows the average body weight for each group throughout the C4-2 xenograft study.

### Example 7

### Results of the Efficacy Study in the LNCAP FGC Xenograft Prostate Cancer Model

The objective of this study was to evaluate the in vivo therapeutic efficacy of the compound of structure (I) in the treatment of a subcutaneous LNCaP FGC human prostate cancer xenograft model. LNCaP clone FGC is an androgen-dependent prostate cancer cell line established from prostate cancer cell line LNCaP. For the LNCaP clone FGC model, efficacy was evaluated in male NCG (NOD-Prkdc^{em26Cd52} Il2rg^{em26Cd22} Nju) mice. Each mouse was implanted subcutaneously with an androgen pellet (15 mg/pellet, with testosterone propionate powder from Aladdin Industrial Corporation, USA) at the left flank 1 day before the tumor inoculation. Each mouse was inoculated at the right flank with 1×10⁷ tumor cells (LNCaP, clone FGC cells) in 0.1 ml of PBS mixed with matrigel (1:1). The date of tumor cell inoculation is denoted as day 0.

Castration was performed when mean tumor volume reached approximately 200 mm³. Mice were anesthetized with ketamine/xylazine; surgical castration was performed via a midline scrotal incision allowing bilateral access to the hemiscrotal contents; after exposing each testicle, a 6-0 Vicryl suture was used to ligate the spermatic cord and then remove the testicle; the scrota and skin were then closed with 6-0 Vicryl suture, separately.

The uncastrated mice were randomized when mean tumor volume reached approximately 100-200 mm³. After castration, the randomization was started when the mean tumor volume of castrated mice reached approximately 100-200 mm³. Randomization was performed based on “Matched distribution” method (StudyDirector™ software, version 3.1.399.19).

Mice were enrolled in the study and randomly allocated to study groups as show in Table 5.

TABLE 5
Treatment groups for the LNCaP FGC xenograft study.

| | | | | | | Dosing |
|---|---|---|---|---|---|---|
| | | | | Dose level | | Frequency & |
| Group | Castrated | No. | Treatment | (mg/kg) | ROA | Duration |
| 1 | NO | 6 | Vehicle 1 | 0 | p.o. | QD × 21 days |
| | | | Vehicle 2 | 0 | p.o. | QD × 21 days |
| 2 | Yes | 10 | Vehicle 1 | 0 | p.o. | QD × 21 days |
| | | | Vehicle 2 | 0 | p.o. | QD × 21 days |
| 3 | Yes | 10 | Vehicle 1 | 0 | p.o. | QD × 21 days |
| | | | Enzalutamide | 30 | p.o. | QD × 21 days |
| 4 | Yes | 10 | Vehicle 1 | 0 | p.o. | QD × 21 days |
| | | | Abiraterone | 50 | i.p. | QD × 21 days |
| 5 | Yes | 10 | Vehicle 2 | 0 | p.o. | QD × 21 days |
| | | | Docetaxel | 10 | i.p. | Q7D × 3 weeks |
| 6 | Yes | 10 | Vehicle 2 | 0 | p.o. | QD × 21 days |
| | | | Cmpd Str (I) | 1.25 | p.o. | BID × 21 days |
| 7 | Yes | 10 | Vehicle 2 | 0 | p.o. | QD × 21 days |
| | | | Cmpd Str (I) | 10 | p.o. | Q7D × 3 weeks |
| 8 | Yes | 10 | Cmpd Str (I) | 1.25 | p.o. | BID × 21 days |
| | | | Enzalutamide | 30 | p.o. | QD × 21 days |
| 9 | Yes | 10 | Cmpd Str (I) | 1.25 | p.o. | BID × 21 days |
| | | | Docetaxel | 10 | i.p. | Q7D × 3 weeks |

Each treatment was administered at a dosing volume of 5 μL/g, beginning one day post-grouping (day 1), and was continued for 21 days (or 22 days for groups with treatment at Q7D). For groups with treatment at Q7D×3 weeks, dosing was performed on days 1, 8, and 15 post-randomization, and the study was terminated on day 22 post-randomization. The combined treatment intervals were each 0 h, and the interval for BID was 8h. The study was terminated 49 days post-inoculation for efficacy.

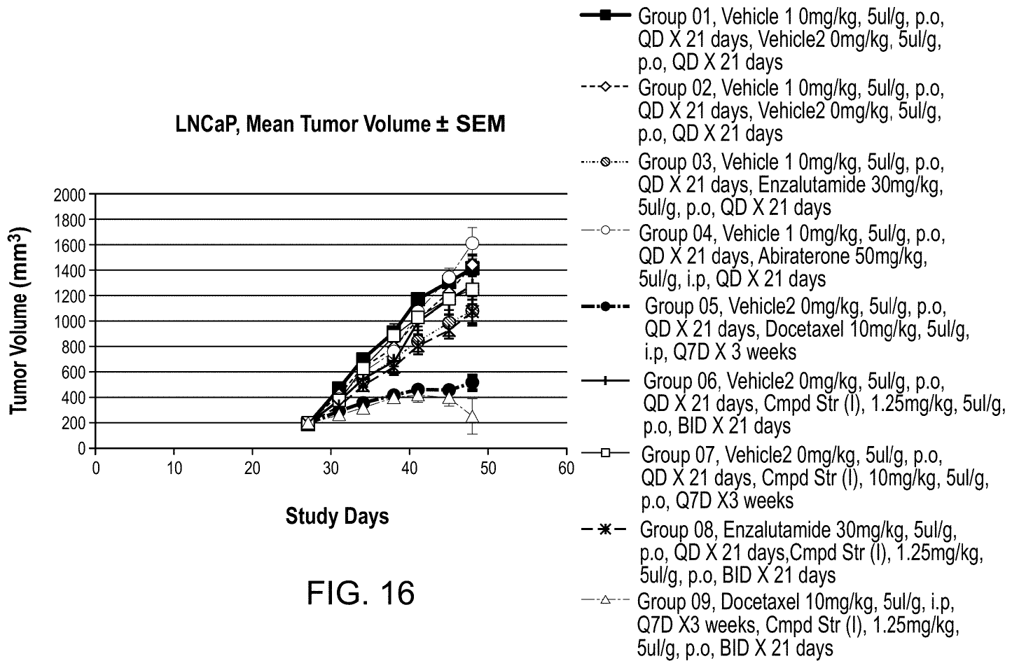

Tumor volumes were measured twice per week after randomization in two dimensions using a caliper, and the volume was expressed in mm³ using the formula: “V=(L×W×W)/2,” where V is tumor volume, L is tumor length (the longest tumor dimension) and W is tumor width (the longest tumor dimension perpendicular to L). FIG. 16 shows the average tumor volume for each group throughout the LNCaP xenograft study.

Tumors were harvested at the end of study (24 hours post final dose for QD treatment groups; 12 hours post final dose for BID treatment groups and 6 days post final dose for Q7D treatment groups): for each tumor, ½ was utilized for snap freezing, and ½ was utilized for FFPE.

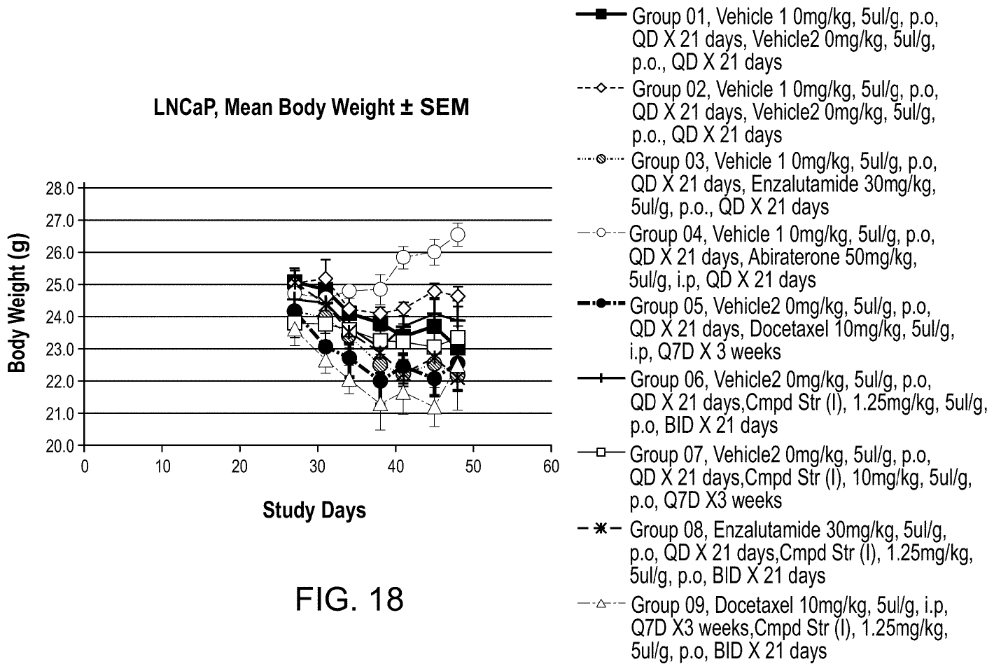

The body weight of all animals was monitored throughout the study. FIG. 18 shows the average body weight for each group throughout the LNCaP xenograft study.

### Example 8

### Alvocidib Inhibits RNA Pol II Phosphorylation in 22Rv1 Prostate Cancer Cell Line

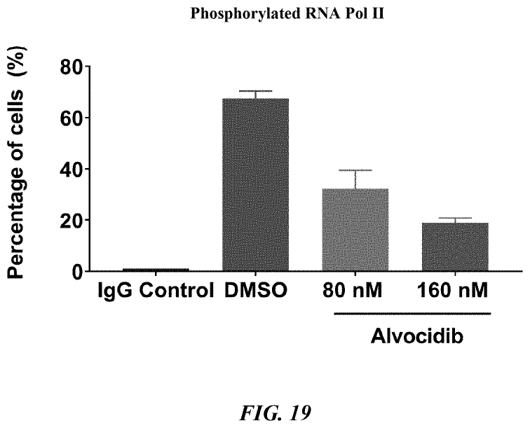

To evaluate the effects of alvocidib on serum-stimulated prostate cancer cells, RNA Polymerase (Pol) II phosphorylation was assessed by flow cytometry. The prostate cancer cells, 22Rv1 cells, were treated with DMSO or alvocidib (80 nM or 160 nM) for three hours, and were serum stimulated (10%) one hour prior to sample collection. Goat anti-rabbit IgG H&L was used as an isotype control. FIG. 19 shows the percentage of stained cells after alvocidib treatment when compared to DMSO control by flow cytometry. 22Rv1 cells showed a dose-dependent reduction in RNA Pol II phosphorylation staining in the flow cytometry assay when treated with alvocidib.

### Example 9

### Alvocidib Inhibits PSA Expression in Androgen-Independent Prostate Cancer Cell Lines, LNCaP and VCaP

PSA protein levels were measured in prostate cancer cell lines, VCaP and LNCaP, after 48-hour treatment with 25 nM or 100 nM alvocidib. On the day of the treatment, the media was replaced with 10 mL of fresh media. VCaP and LNCaP cells were treated with 25 nM or 100 nM alvocidib for 48 hours. Cells were collected by scraping, washed in PBS, and lysed using sonication in RIPA buffer supplemented with protease and phosphatase inhibitors. 30 ng of protein was loaded into a 4-12% gel, and transferred to a nitrocellulose membrane. Blocking, anti-actin, and secondary staining was done in 5% milk. Anti-PSA antibody was diluted in BSA.

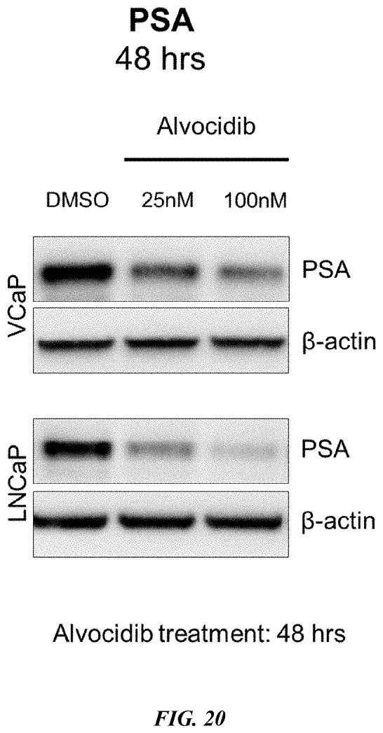

FIG. 20 shows that alvocidib treatment inhibited PSA expression in VCaP and LNCaP prostate cell lines at protein level after 48 hours when compared to vehicle group.

### Example 10

### Alvocidib Induces Cell Death in Prostate Cancer Cell Lines

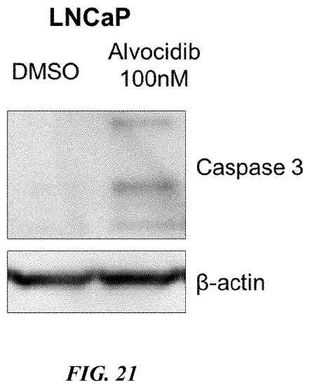

LNCaP cells were treated with 100 nM alvocidib for 48 hours in regular 10% serum conditions. Cells were collected by scraping, washed in PBS, and lysed using sonication in RIPA buffer supplemented with protease and phosphatase inhibitors. 30 ng of protein was loaded into a 4-12% gel, and transferred to a PVDF membrane. Blocking and staining was performed in 5% milk. FIG. 21 shows that alvocidib treatment induced cell death, as indicated by caspase 3 cleavage, using anti-cleaved caspase 3 antibody. Anti-actin was used as a loading control.

### Example 11

### Oral Alvocidib Prodrug is Retained in Plasma and Tumor, and Inhibits MCL1 and Tumor Growth in a Pc-3 Mouse Xenograft Model

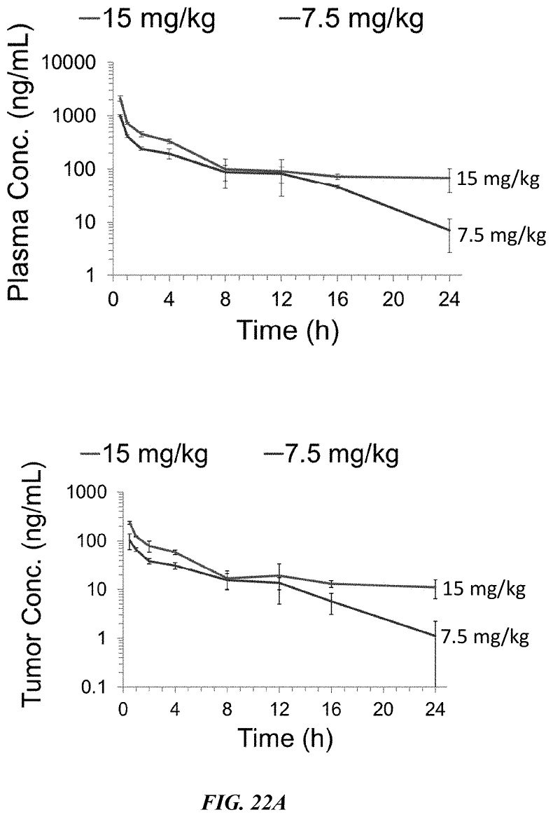

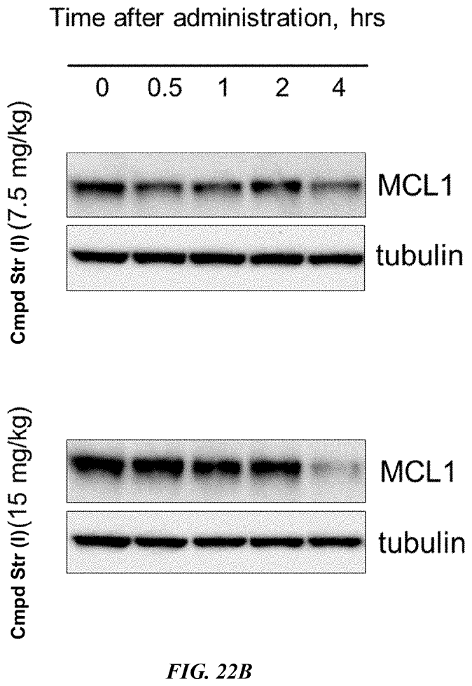

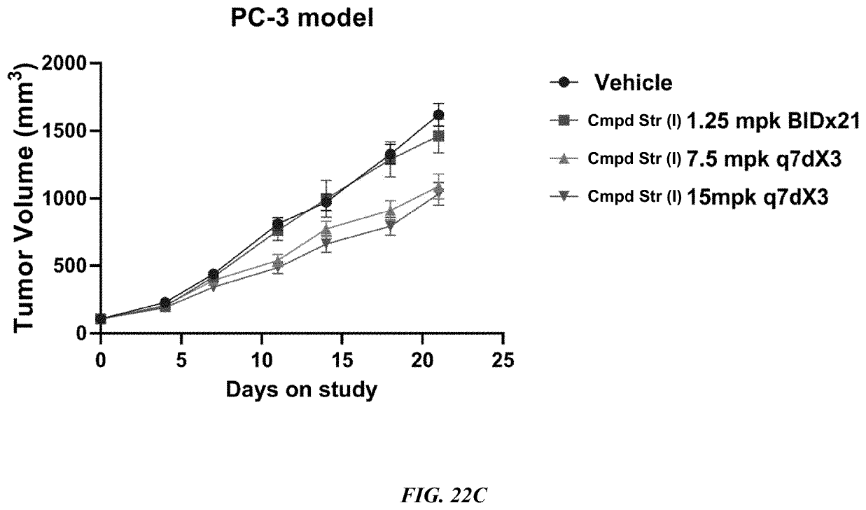

A PK/PD analysis of oral alvocidib prodrug, compound of structure (I), was conducted in a PC-3 mouse xenograft model. PC-3 prostate tumor cells were maintained in vitro as a monolayer culture in Ham's F12K medium supplemented with 10% fetal bovine serum at 37° C. in an atmosphere of 5% CO₂ in air. The cells in an exponential growth phase were harvested and counted for tumor inoculation. Each mouse was inoculated subcutaneously at the right flank region with PC-3 prostate tumor cells (5×10⁶ cells) in 0.1 ml of PBS for tumor development. When the mean tumor size reached approximately 300 mm³, the mice were randomized and divided into study groups. Compound of structure (I) was administered at 7.5 or 15 mg/kg by oral gavage, with tumor and plasma collected at 0, 0.5, 1, 2, 4, 8, 12, 16, and 24 hours post-dosing for each dose level. Concentrations of compound of structure (I) and alvocidib in tumor and plasma were determined by LC-MS/MS for pharmacokinetic analysis. FIG. 22A shows that alvocidib was retained in plasma and tumor tissues 24 hours after administration of the compound of structure (I).

Additional tumor samples from the study were homogenized by bead homogenizer and lysed using RIPA buffer with protease and phosphatase inhibitors. Samples from each treatment group were pooled and protein expression analyzed by Western blot using anti-MCL1 antibody and anti-O-tubulin antibody as a loading control. FIG. 22B shows that the compound of structure (I) inhibited MCL1 in PC-3 tumors at 4 hours after oral administration, as shown by Western blot.

The effects of the compound of structure (I) on tumor growth in the PC-3 mouse xenograft model were also assessed. PC-3 prostate tumor cells were maintained in vitro as a monolayer culture in Ham's F12K medium supplemented with 10% fetal bovine serum at 37° C. in an atmosphere of 5% CO₂ in air. The cells in an exponential growth phase were harvested and counted for tumor inoculation. Each mouse was inoculated subcutaneously at the right flank region with PC-3 prostate tumor cells (5×10⁶ cells) in 0.1 ml of PBS for tumor development. When the mean tumor size reached approximately 100 mm³, the mice were randomized and divided into study groups. Compound of structure (I) was administered at 1.25 mg/kg twice daily (BID) for 21 days, or 7.5 mg/kg or 15 mg/kg once weekly (q7d×3) by oral gavage. Tumor volumes were measured twice weekly in two dimensions using a caliper, and the volume expressed in mm³ using the formula: V=(L×W×W)/2, where V is tumor volume, L is tumor length (the longest tumor dimension) and W is tumor width (the longest tumor dimension perpendicular to L). FIG. 22C shows that the compound of structure (I), administered orally, inhibited tumor growth in the PC-3 mouse xenograft model.

### Example 12

### Phase I, Pharmacokinetic (PK) and Pharmacodynamic (PD), Dose-Escalation Study of Oral Compound of Structure (I) Administered to Patients with Advanced Solid Tumors

Patients with advanced metastatic or progressive solid tumors who were refractory to, or intolerant of, established therapy known to provide clinical benefit for their condition were enrolled. Cohorts of 3-6 patients each received escalating doses of compound of structure (I) using a modified Fibonacci dose escalation approach. Once the optimal dose has been established, additional patients may be enrolled to confirm safety and to explore efficacy. Twenty additional patients will be enrolled in an expansion cohort at the maximum tolerated dose (MTD).

This is an ongoing Phase 1, open-label, dose-escalation, safety, PK and PD study. The proposed starting dose and schedule for oral compound of structure (I) was a 1-mg flat dose once daily (QD) for 14 days followed by a 7-day drug-free recovery period (each cycle=21 days). In the absence of dose-limiting toxicities (DLTs) in the first cohort of at least 3 patients, the dose was increased using a modified Fibonacci dose escalation scheme, and BID dosing commenced according to the dose escalation schedule described in Table 8. The first patient in cohort 6 has been enrolled at 8 mg compound of structure (I) BID. The baseline demographics of the first 14 patients enrolled in the study are described in Table 6.

TABLE 6
Baseline Demographics (N = 14 ITT)

| | |
|---|---|
| Median Age | 65 (45-78) |
| Median Number of Prior Therapies | 4 (1-10) |
| ECOG Score | ECOG 0: 1 (7%) |
| | ECOG 1: 13 (93%) |
| Primary Tumor Type | Bladder: 2 |
| | Pancreas: 2 |
| | Colorectal: 2 |
| | Kidney: 1 |
| | NSCLC: 1 |
| | Prostate: 1 |
| | Sarcoma: 1 |
| | Skin: 1 |
| | Thyroid: 1 |
| | Testicular: 1 |
| | Vulvar: 1 |
| Gender | Male: 8 (57.1%) |
| | Female: 6 (42.9%) |

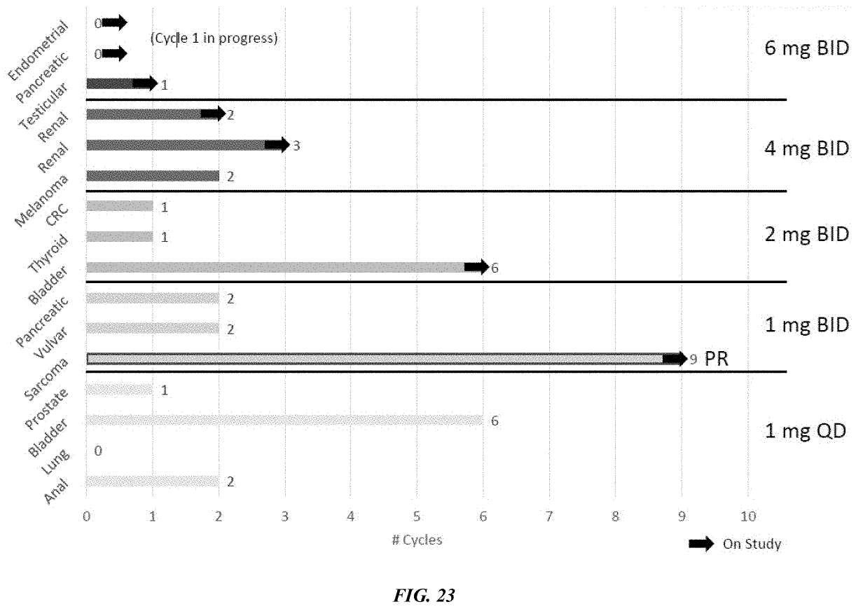

FIG. 23 is a graph depicting completed cycles on the study through Cohort 5.

To date, there is no unexplained toxicity, and no evidence of dose-limiting diarrhea or neutropenia. The treatment-emergent adverse events of grade ≥3 observed thus far are reported in Table 7.

TABLE 7
Treatment-emergent adverse events grade ≥3.

| MedDRA Preferred Term | Grade 3 | Grade 4 | Grade 5 |
|---|---|---|---|
| Anemia | 1 (7%) | — | — |
| Chest Pain | 1 (7%) | — | — |
| Pain | 1 (7%) | — | — |
| Swelling | 1 (7%) | — | — |
| Malignant Pleural Effusion | 1 (7%) | — | — |
| Haematuria | 1 (7%) | — | — |
| Hypoxia | 1 (7%) | — | — |
| Hypotension | 1 (7%) | — | — |

Sequential cohorts of 3 patients will continue to be treated with escalating doses according to Table 8 until the MTD is established.

TABLE 8

| Dose | Proposed | | Total | Increment from | No. of Patients |
|---|---|---|---|---|---|
| Level | Daily Dose | | Daily Dose | Previous Dose^{a} | Per Cohort |
| −1^{b} | 1 | mg QOD | N/A | −50% | 3-6 |
| 1 ^{c} | 1 | mg QD | 1 mg | Starting Dose | 3-6 |
| 2 | 1 | mg BID | 2 mg | 100% | 3-6 |
| 3 | 2 | mg BID | 4 mg | 100% | 3-6 |
| 4 | 4 | mg BID | 8 mg | 100% | 3-6 |
| 5 | 6 | mg BID | 12 mg | 50% | 3-6 |
| 6 | 8 | mg BID | 16 mg | 33% | 3-6 |
| 7^{d} | 11 | mg BID | 22 mg | 33% | 3-6 |

^{a}It is possible for additional and/or intermediate dose levels to be added during the course of the study,
^{b}Dose level −1 represents a treatment dose for patients requiring a dose reduction from the starting dose level. It will also serve as a lower dose level if the Starting Dose level is initially associated with unexpected or unacceptable toxicity. Please note that the dosing in this instance is a single morning dose every other day (QOD) (no evening doses required).
^{c} Please note that the dosing in Cohort 1 is a single daily (QD) morning dose (no evening dose required),
^{d}If clinically indicated, dose levels higher than 11 mg BID may be investigated.

If a DLT is observed in 1 of 3 patients at a given dose level, up to 3 additional patients will be enrolled and treated at that dose level. When up to 3 additional patients are added to a given dose level, if only 1 out of those 6 patients experiences a DLT, the dose will be increased to the next dose level. If ≥2 out of 3-6 patients at a dose level experience DLTs, the dose will be decreased to the previous (lower) dose level and 3 additional patients will be enrolled at that dose level.

If 0 or 1 patient in any of the 6 patients experience a DLT, but the next higher dose level has already been studied, then the current dose will be declared the MTD and the study will advance to the expansion cohort.

The MTD is defined as the dose at which ≤1 of 6 patients experience a DLT during Cycle 1 with the next higher dose having at least 2 of 3 to 6 patients experiencing a DLT during Cycle 1.

Once the MTD has been established, 20 additional patients will be enrolled at the MTD. Data collected from patients enrolled at the MTD will be used to confirm safety, explore potential biomarkers, and evaluate potential signals of compound of structure (I) activity.

All patients may continue to receive compound of structure (I) in 21-day cycles (14 days of active treatment) at the same dose given during Cycle 1 until they experience unacceptable toxicity or unequivocal disease progression. Patients in the 20-patient expansion cohort may receive compound of structure (I) at the MTD in 28-day cycles including 21 days of active treatment followed by a seven-day drug-free recovery period, if tolerated. No intra-patient escalation of the compound of structure (I) dose is permitted during the escalation phase until MTD is established.

Patients met all of the following inclusion criteria:
1. Have a histologically confirmed diagnosis of advanced metastatic or progressive solid tumor excluding tumor types with rapid cell turnover, i.e., small cell cancer (lung and extra pulmonary), inflammatory breast cancer (IBC), medulloblastoma, neuroblastoma and melanoma with extensive liver metastasis (≥50% of the liver involved; patients with melanoma and metastasis to <50% of the liver were eligible)
2. Be refractory to, or intolerant of, established therapy known to provide clinical benefit for their condition
3. Have one or more tumors measurable or evaluable as outlined by the Response Evaluation Criteria in Solid Tumors (RECIST) v1.1
4. Have an Eastern Cooperative Oncology Group (ECOG) performance status of ≤1
5. Have a life expectancy ≥3 months
6. Be ≥18 years of age
7. Have a negative pregnancy test (if female of childbearing potential)
8. Have acceptable liver function:
   a) Bilirubin ≤1.5× upper limit of normal (ULN) (unless associated with Gilbert syndrome)
   b) Aspartate aminotransferase (AST/SGOT), alanine aminotransferase (ALT/SGPT) and alkaline phosphatase ≤2.5×ULN*
      * if liver metastases were present, then 3×ULN was allowed
9. Have acceptable renal function: calculated creatinine clearance ≥30 mL/min
10. Have acceptable hematologic status:
   c) Granulocyte ≥1500 cells/mm³
   d) Platelet count ≥100,000 (plt/mm³)
   e) Hemoglobin ≥8 g/dL
11. Have acceptable coagulation status:
   f) Prothrombin time (PT) within 1.5× normal limits
   g) Activated partial thromboplastin time (aPTT) within 1.5× normal limits
12. Be nonfertile or agree to use an adequate method of contraception. Sexually active patients and their partners used an effective method of contraception (hormonal or barrier method of birth control; or abstinence) prior to study entry and for the duration of study participation and for at least 3 months (males) and 6 months (females) after the last study drug dose.
13. Have read and signed the Institutional Review Board (IRB)-approved informed consent form (ICF) prior to any study-related procedure.

Patients meeting any one of the following exclusions criteria were prohibited from participating in the study:
1. History of congestive heart failure (CHF); cardiac disease, myocardial infarction within the past 6 months prior to Cycle 1/Day 1; left ventricular ejection fraction (LVEF) <45% by echocardiogram (ECHO), unstable arrhythmia, or evidence of ischemia on electrocardiogram (ECG) within 14 days prior to Cycle 1/Day 1
2. Have a corrected QT interval (using Fridericia's correction formula) (QTcF) of >450 msec in men and >470 msec in women
3. Have a seizure disorder requiring anticonvulsant therapy
4. Presence of symptomatic central nervous system metastatic disease or disease that requires local therapy such as radiotherapy, surgery, or increasing dose of steroids within the prior 2 weeks
5. Have severe chronic obstructive pulmonary disease with hypoxemia (defined as resting O₂ saturation of ≤90% breathing room air)
6. Have undergone major surgery within 2 weeks prior to Cycle 1/Day 1
7. Have active, uncontrolled bacterial, viral, or fungal infections, requiring systemic therapy
8. Are pregnant or nursing
9. Received treatment with radiation therapy, surgery, chemotherapy, or investigational therapy within 28 days or 5 half-lives, whichever occurs first, prior to study entry (6 weeks for nitrosoureas or mitomycin C)
10. Are unwilling or unable to comply with procedures required in this protocol
11. Have known infection with human immunodeficiency virus, hepatitis B, or hepatitis C. Patients with history of chronic hepatitis that is currently not active are eligible
12. Have a serious nonmalignant disease (eg, hydronephrosis, liver failure, or other conditions) that could compromise protocol objectives in the opinion of the Investigator and/or the Sponsor
13. Are currently receiving any other investigational agent
14. Have exhibited allergic reactions to a similar structural compound, biological agent, or formulation
15. Have malabsorption conditions (e.g., Crohn's disease) or have undergone significant surgery to the gastrointestinal tract that could impair absorption or that could result in short bowel syndrome with diarrhea due to malabsorption.

DLT was defined as any one of the following events observed in cycle 1, regardless of investigator attribution, unless there was a clear alternative explanation:
1. Grade 3 or greater febrile neutropenia
2. Grade 4 neutropenia for ≥7 consecutive days
3. Grade 4 thrombocytopenia or Grade 3 thrombocytopenia with clinically significant bleeding or that requires a platelet transfusion
4. Grade 3 or 4 nonhematologic AEs will be considered dose limiting, regardless of duration aside from the specific parameters described herein
5. Grade 4 nausea, vomiting, or diarrhea, regardless of duration 6. Dosing delays >1 week due to treatment-emergent adverse events (TEAEs) or related severe laboratory test values
7. Any AST and ALT elevation >3×ULN (if baseline value was normal) or ≥3× the baseline value (if baseline value was abnormal) accompanied by serum bilirubin levels >2×ULN
8. Any Grade ≥3 electrolyte disturbances (eg, hyperkalemia, hypophosphatemia, hyperuricemia) that do not resolve within <72 hours
9. Any Grade ≥3 elevations in creatinine
10. Any Grade 5 toxicity

Plasma PK parameters of compound of structure (I) and alvocidib were evaluated in Cohorts 1-5 at specific timepoints during the study. Blood was collected from patients in Cohorts 1-5 according to the pharmacokinetic sampling schedule described in Table 9.

TABLE 9
Pharmacokinetic Sampling Schedule

| | Cycle No. | | | | | | | |
|---|---|---|---|---|---|---|---|---|
| | CYCLE 1 | | | | | | CYCLE 2 | |
| Time | AM | PM ^{a} | AM | AM | PM ^{a} | AM | AM | End |
| Points | Day | Day | Day | Day | Day | Day | Day | of |
| (hrs) | 1 | 1 | 2 | 14 | 14 | 15 | 1 | Study |
| 0 (pre-dose) | X | | X ^{b} | X ^{b} | | X ^{b} | X | X |
| 0.5 | X | X | | X | X | | | |
| 1 | X | X | | X | X | | | |
| 2 | X | X | | X | X | | | |
| 4 | X | | | X | | | | |
| 8 ^{c} | X | | | X | | | | |

^{a} No evening (PM) samples were collected from patients enrolled in the first dose cohort receiving compound structure (I) as a once daily (QD) morning dose.
^{b} Approximately 24 hours after taking the previous days' morning dose and prior to taking current days' dose (i.e., sampling on Cycle 1/Day 2 would be performed 24 hrs after taking the morning dose on Day 1 and before taking the morning dose on Day 2)
^{c} The 8-hour samples were collected just prior to taking that day's evening dose (for patients receiving compound of structure (I) BID), or 8 hours after taking that day's dose (for patients receiving compound of structure (I) QD).

PK parameters were estimated using standard noncompartmental methods. Actual sample collection times were used rather than scheduled collection times. Plasma concentrations below the limit of quantification were treated as 0. Imbedded missing plasma concentrations (e.g., missing values between two observed values) were estimated using linear extrapolation. This is consistent with using the trapezoidal rule to calculate AUC. Other missing plasma concentrations were excluded from calculations to estimate PK parameters.

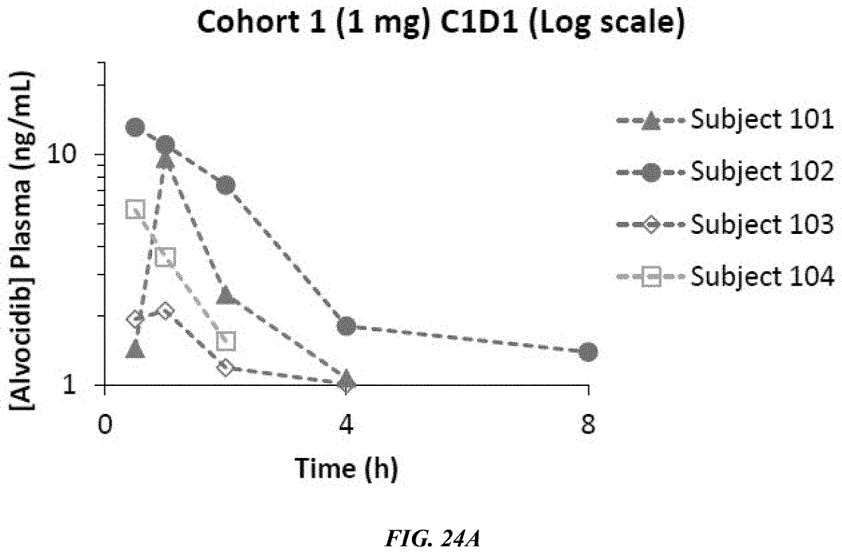

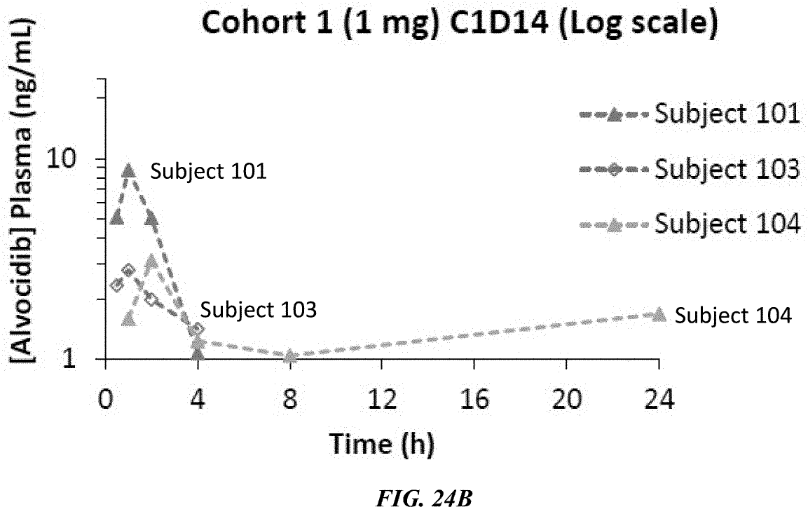

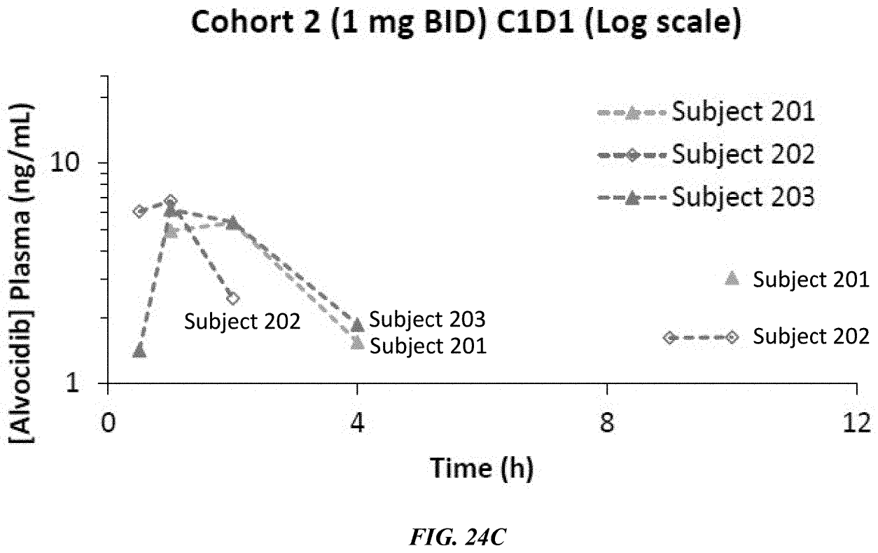

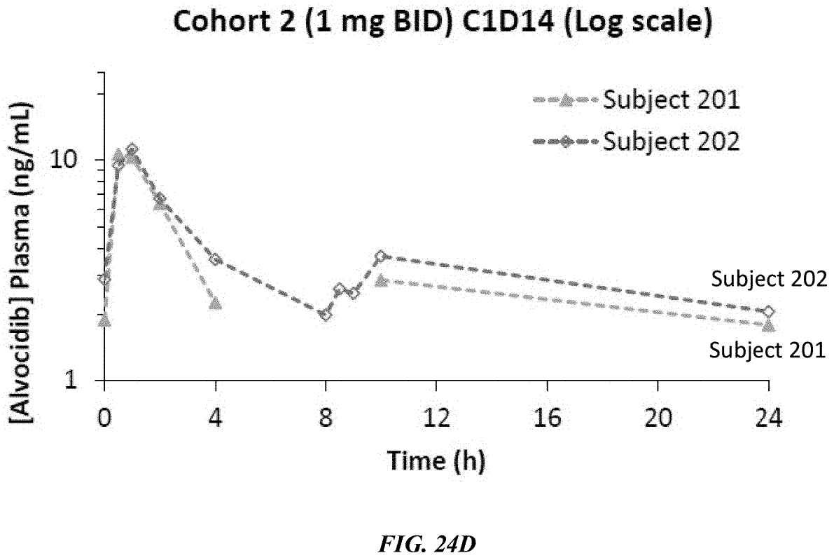

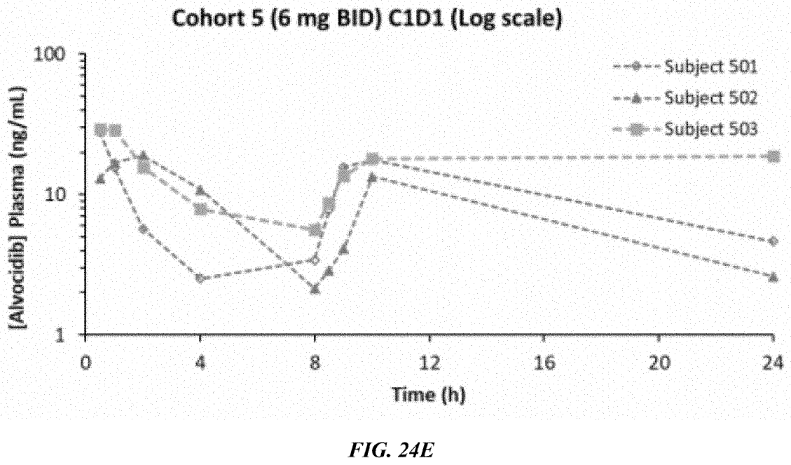

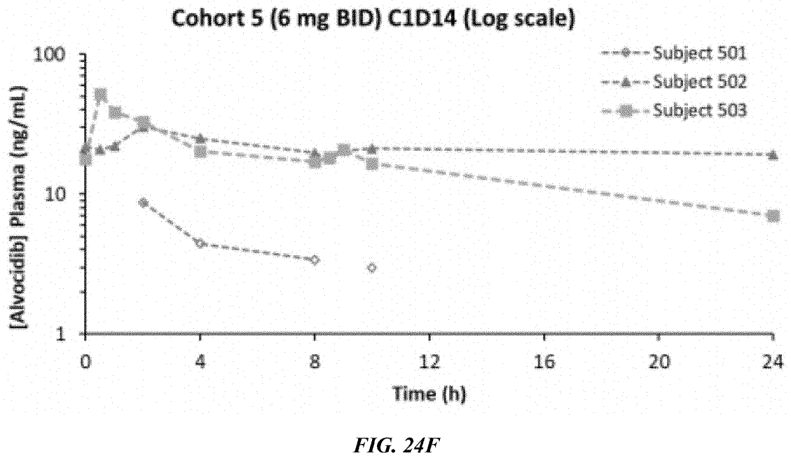

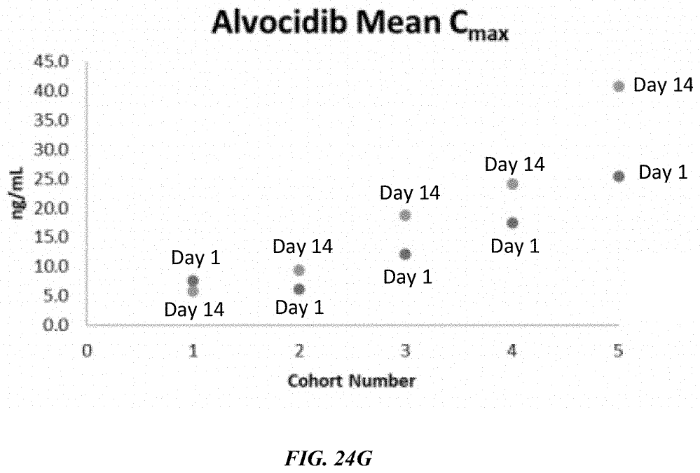

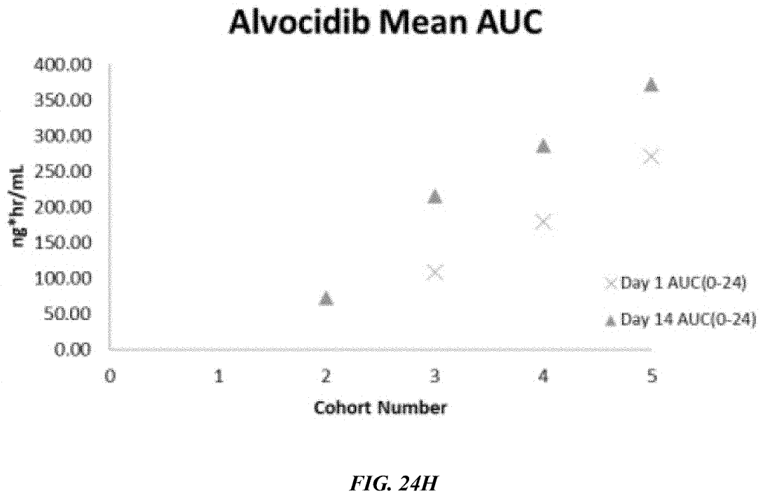

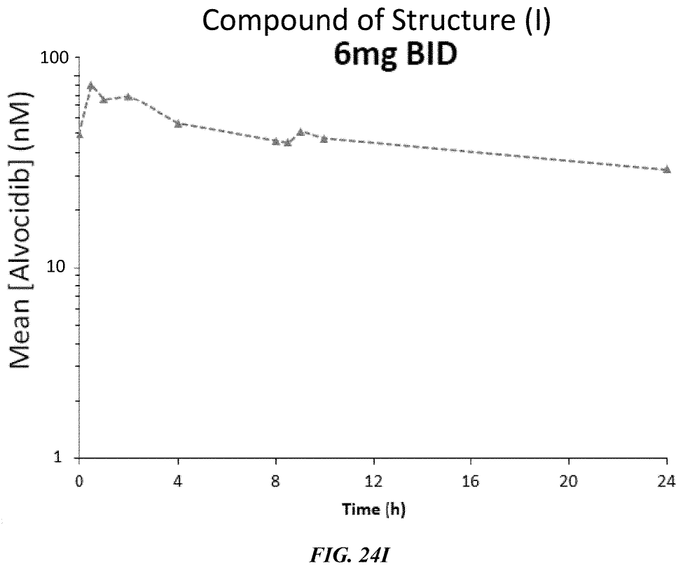

FIGS. 24A and 24B are graphs of plasma alvocidib concentration (ng/mL) versus time, and show the concentration of alvocidib in the plasma of patients in Cohort 1 on days 1 and 14, respectively, following daily oral QD dosing with a 1-mg strength capsule containing Formulation No. 401-01. Subject 104 showed some accumulation of alvocidib after 24 hours on day 14. Subject 102 was discontinued prior to day 14 dosing.

FIGS. 24C and 24D are graphs of plasma alvocidib concentration (ng/mL) versus time, and show the concentration of alvocidib in the plasma of patients in cohort 2 on days 1 and 14, respectively, following daily oral BID dosing with a 1-mg strength capsule containing Formulation No. 401-01. Only alvocidib was detectable at 1 mg BID, and no compound of structure (I) was detected at any timepoint for any sample. No drug was detectable (less than 1.0 ng/mL of alvocidib) by 8 hours and again at 24 hours in any subject on day 1. However, there was detectable accumulation of alvocidib on day 14 for subjects 201 and 202 (average=2.39 ng/mL), suggesting that BID dosing helped to maintain drug levels by day 14.

FIGS. 24E and 24F are graphs of plasma alvocidib concentration (ng/mL) versus time, and show the concentration of alvocidib in the plasma of patients in Cohort 5 on days 1 and 14, respectively, following daily oral BID dosing with 6 mg of Formulation No. 401-01. Table 10 reports Tₘₐₓ, Cₘₐₓ and AUC₍₀₋₂₄₎ of alvocidib for patients in Cohort 5 on days 1 and 14 of cycle 1.

TABLE 10
Cohort 5 6 mg BID

| | Cycle 1 | | | | | |
|---|---|---|---|---|---|---|
| | Day 1 | | | Day 14 | | |
| Subject | Tₘₐₓ | Cₘₐₓ | AUC₍₀₋₂₄₎ | Tₘₐₓ | Cₘₐₓ | AUC₍₀₋₂₄₎ |
| (#) | (Hours) | (ng/mL) | (ng*h/mL) | (Hours) | (ng/mL) | (ng*h/mL) |
| 501 | 0.5 | 28 | 230 | 0.5 | 40.2 | 192 |
| 502 | 2 | 19 | 208 | 2 | 30.4 | 516 |
| 503 | 0.5 | 29.3 | 376 | 0.5 | 51.7 | 407 |
| Mean | 1.0 | 25.4 | 271.3 | 1.0 | 40.8 | 371.7 |
| SD | 0.9 | 5.6 | 91.3 | 0.9 | 10.7 | 164.9 |

FIG. 24G is a graph of alvocidib (ng/mL) versus cohort, and shows the mean Cₘₐₓ of alvocidib on day 1 and day 14 following daily oral QD dosing with a 1-mg strength capsule containing Formulation No. 401-01. FIG. 24H is a graph of alvocidib (ng*hr/mL) versus cohort, and shows the area under the curve (AUC) of alvocidib on day 1 (AUC₀₋₈) and day 14 (AUC₀₋₈ and AUC₀₋₂₄) following daily oral BID dosing with a 1-mg strength capsule containing Formulation No. 401-01. There was no detectable compound of structure (I) at any timepoint. Cohort 2 showed marked increase in average Cₘₐₓ and AUC from day 1 to day 14, illustrating the impact of BID versus QD dosing. The Cₘₐₓ for Cohort 5 increased by 46% compared to Cohort 4 on day 1, and by 69% for day 14. The corresponding increase in AUC was 52% on day 1 and 30% on day 14.

FIG. 24I is a graph of mean concentration of alvocidib (nM) versus time, and shows the mean concentration of alvocidib in plasma of Cohort 5 patients over a 24-hour period. By administering alvocidib as compound of structure (I), alvocidib can be given at a lower dose over a longer time, with less toxicity and similar exposure.

### Example 13

### Polymorph Form B

The absolute stereochemistry, the position of the phosphoric acid moiety, as well as the zwitterionic nature of Form B of the compound of structure (II) were determined by single-crystal X-ray diffraction using the following parameters:

Stoe Stadi P. Copper KαI radiation, 40 kV/40 mA; Mythen 1K detector transmission mode, curved monochromator, 0.02° 20 step size, 12 s step time, 1.5-50.5° 20 scanning range with 1° 20 detector step in step-scan mode. Each sample (25-40 mg of powder) was placed between two cellulose acetate foils spaced with a metal washer (0.4 mm thick, 12-mm inner diameter; “sandwich element”). The sandwich element was transferred to a sample holder (SCell) that was sealed with acetate foils. Samples were acquired in ambient air atmosphere and rotated during measurements.

FIG. 25 shows the XRPD diffractogram produced from the XRPD analysis of Form B. Form B crystallizes as an anhydrous molecule without solvent inclusion. Bond distances and angles were all within the expected values. Tabulated data generated for Form B is provided in Table 11.

TABLE 11
Tabulated data from XRPD diffractogram of Form B

| D value | | Intensity | Intensity | |
|---|---|---|---|---|
| (Å) | 2θ | (relative) | (absolute) | FWHM |
| 18.382645 | 4.8032 | 55.17 | 3833 | 0.08 |
| 9.190754 | 9.6155 | 10.48 | 728 | 0.08 |
| 8.157735 | 10.8365 | 93.39 | 6489 | 0.06 |
| 6.471747 | 13.6717 | 49.51 | 3440 | 0.08 |
| 5.956605 | 14.8604 | 73.17 | 5084 | 0.04 |
| 5.524677 | 16.0296 | 11.07 | 769 | 0.06 |
| 5.281129 | 16.774 | 30.9 | 2147 | 0.06 |
| 5.172326 | 17.1295 | 9.19 | 639 | 0.06 |
| 5.080202 | 17.4425 | 17.69 | 1229 | 0.06 |
| 4.984581 | 17.7798 | 58 | 4030 | 0.04 |
| 4.722631 | 18.7747 | 18 | 1250 | 0.06 |
| 4.654062 | 19.0539 | 25.93 | 1802 | 0.04 |
| 4.552517 | 19.483 | 32.41 | 2252 | 0.06 |
| 4.445879 | 19.955 | 100 | 6948 | 0.06 |
| 4.144506 | 21.4226 | 19.68 | 1367 | 0.04 |
| 4.079254 | 21.7694 | 22.77 | 1582 | 0.06 |
| 3.90758 | 22.7383 | 9.77 | 679 | 0.04 |
| 3.867711 | 22.9759 | 28.61 | 1988 | 0.06 |
| 3.72577 | 23.8638 | 22.65 | 1573 | 0.06 |
| 3.6868 | 24.1198 | 25.68 | 1785 | 0.04 |
| 3.616616 | 24.5951 | 48.94 | 3401 | 0.06 |
| 3.512733 | 25.3344 | 7.74 | 538 | 0.06 |
| 3.394705 | 26.2307 | 28.77 | 1999 | 0.06 |
| 3.350985 | 26.5791 | 11.43 | 794 | 0.04 |
| 3.319773 | 26.8337 | 18.57 | 1290 | 0.06 |
| 3.241092 | 27.4977 | 23.54 | 1635 | 0.08 |
| 3.142734 | 28.376 | 15.33 | 1065 | 0.06 |
| 3.055085 | 29.208 | 12.7 | 882 | 0.08 |
| 2.973274 | 30.0303 | 10.91 | 758 | 0.14 |
| 2.920812 | 30.5827 | 6.32 | 439 | 0.06 |
| 2.830324 | 31.5856 | 9.24 | 642 | 0.08 |
| 2.748258 | 32.5546 | 6.82 | 474 | 0.04 |
| 2.731945 | 32.7544 | 11.76 | 817 | 0.06 |
| 2.703955 | 33.1032 | 6.09 | 423 | 0.06 |
| 2.64826 | 33.8201 | 10.18 | 707 | 0.04 |
| 2.640271 | 33.9255 | 14.93 | 1037 | 0.06 |
| 2.60744 | 34.3659 | 16.09 | 1118 | 0.06 |
| 2.587021 | 34.6457 | 11.27 | 783 | 0.06 |
| 2.540227 | 35.3046 | 5.6 | 389 | 0.06 |
| 2.424055 | 37.0566 | 5.24 | 364 | 0.06 |
| 2.216436 | 40.6738 | 7.03 | 488 | 0.06 |
| 2.125602 | 42.4942 | 10.93 | 760 | 0.06 |
| 2.078432 | 43.5072 | 4.98 | 346 | 0.14 |
| 2.042561 | 44.3113 | 12.61 | 876 | 0.1 |
| 2.008764 | 45.0975 | 5.76 | 400 | 0.08 |
| 1.950189 | 46.5303 | 5.84 | 406 | 0.14 |

DSC was performed using a TA Q200/Q2000DSC from TA Instruments using a ramp method and a crimped, aluminum sample pan at 25° C. The heating rate was 10° C./minute, and the purge gas was nitrogen. FIG. 26 shows the differential scanning calorimetry output of heat flow plotted as a function of temperature for polymorph Form B.

Form B can be synthesized according to the procedure depicted in Scheme 1 and described below.

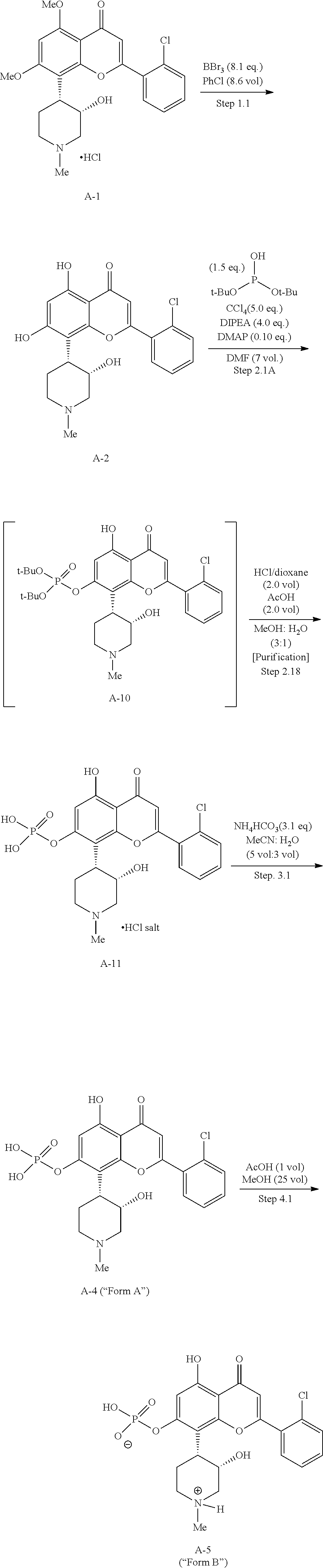

### Scheme 1.

Step 1.1: To a clean and dry, three-necked, round-bottomed flask (RBF) (3 L) was added A-1 (90 g, 0.192 mol) and chlorobenzene (774 ml) at room temperature. To the reaction flask was slowly added BBr₃ (391.5 g) at room temperature. After completion of BBr₃ addition, the temperature of the reaction mixture was slowly raised to 80-83° C., and the reaction mixture was stirred at the same temperature for 10 hours. The reaction mixture temperature was further raised to 100-103° C., and the reaction mixture was maintained at 100-103° C. for 5 hours. The reaction progress was monitored by TLC and HPLC. After completion of the reaction, HBr and methyl bromide was removed at room temperature by nitrogen bubbling into the reaction mixture, while maintaining the vigorous stirring. The reaction mixture was slowly quenched with a mixture of methanol (180 ml)/water (90 ml) (270 ml), followed by methanol (180 ml). The solvent was removed under atmospheric distillation at 25-50° C. to reach the target reaction mass volume of 12 volumes (vol). Then, the reaction mixture pH was adjusted to 3.0±1 using sodium hydroxide solution (48.8 g dissolved in 135 ml of DM water) at 50-55° C. Again, the solvent was removed under atmospheric distillation at 50-100° C. to reach the target reaction volume of 12 vol. Then, the pH of the reaction mixture was adjusted to pH 8.1±0.2 using sodium hydroxide solution (8.5 g dissolved in 87 ml of DM water) at 50° C. followed by slow addition of water with constant stirring at 50° C. for 1 hour. The reaction mixture was slowly allowed to come to room temperature and maintained at room temperature for 3 hours. The resulting solid was filtered and washed with a mixture of methanol (315 ml)/water (135 ml) (3×450 ml) followed by water (5×450 ml). The solid was dried in a vacuum oven at 50-55° C. for 48 hours to obtain A-2 as a yellow solid (70 g, 90%). HPLC Purity: 99.72%.

Step 2.1A: To a clean and dry, three-necked RBF (3 L) was added A-2 (35.0 g, 0.087 mol) and DMF (245 ml), at room temperature, under nitrogen atmosphere. Then, DMAP (1.06 g, 0.0086 mol) followed by CCl₄ (66.5 g 0.434 mol) were added to the reaction mixture at room temperature. To the reaction mixture di-tertiary butyl phosphite (25.5 g, 0.131 mol) was added at room temperature. The reaction mixture was stirred at room temperature under nitrogen atmosphere for 24 hours. The reaction progress was monitored by HPLC. The reaction mixture was cooled to 0-5° C., and was quenched with slow addition of DM water (1950 ml) for 30 minutes at 0-5° C. Then, chloroform (1627.5 ml) was added to the reaction mixture, and the reaction mixture was stirred at 0-5° C. for 10 minutes. The organic layer was separated and dried over sodium sulfate. The solvent was removed under reduced pressure, while maintaining the bath temperature below 45° C. The resulting residue was co-distilled with toluene (4×175 ml). The residue was kept under high vacuum for 45 minutes to obtain A-10 as a pale yellow residue. (51.0 g, 98.5%). HPLC Purity: 91.48%.

Step 2.1B: To a clean and dry RBF (1 L) was added A-10 (51.0 g, 0.0858 mol) and acetic acid (102 ml) at room temperature. Then, 4N HCl solution in 1,4-dioxane (102 ml) was added dropwise at 25-30° C. The reaction mixture was stirred at 25-30° C. for 40 minutes. The reaction progress was monitored by TLC. After completion of the reaction, toluene (2×510 ml) was added to the reaction mixture under stirring, and the reaction mixture was maintained for 5 minutes. The stirring was stopped, and the solids in the reaction mixture were allowed to settle at 25-30° C. for 5 minutes. The solvent was decanted to obtain the semi-solid. The semi-solid was co-distilled with toluene (3×123 ml) to obtain pale yellow solid. The resulting pale yellow solid was taken into a clean RBF, and methanol was added (123 ml) followed by dropwise addition of water (41 ml) at 25-30° C. The reaction mixture was stirred at 25-30° C. for 2 hours to obtain pale yellow solid. The resulting solid was filtered and vacuum dried for 10 minutes to obtain A-11 as a pale yellow solid (36.5 g, 82%). HPLC Purity: 97.03%. This material was directly taken into Step 3.1 without further drying.

Step 3.1: To a clean and dry, three-necked, 500 ml RBF was added A-11 (34.0 g, 0.066 mol) and ACN (51 ml). To the reaction mixture was dropwise added ammonium bicarbonate solution (16.2 g dissolved in 170 ml of DM water) under stirring at 25-30° C. for 30 minutes. Again, ACN (51 ml) was slowly added at 25-30° C. for 30 minutes. The reaction mixture was cooled to 10-15° C. and stirred at 10-15° C. for 60 minutes. The resulting solid was filtered and washed with ACN (102 ml). The solid was dried in a vacuum oven at 25-30° C. for 16 hours to obtain A-4 as a pale yellow solid (28.5 g, 90.10%). HPLC Purity: 99.68%.

Step 4.1: To a clean and dry, 500-ml, three-necked RBF was added A-4 (7.5 g, 0.015 mol) and methanol (187.5 ml) at room temperature. To the reaction mixture was slowly added acetic acid (7.5 ml, 1.0 vol) at 50° C., under nitrogen atmosphere. The reaction mixture was stirred at 50° C. for 1 h, under nitrogen atmosphere. The reaction mixture was cooled to room temperature and stirred for 2 h. The solid was filtered and dried under vacuum to obtain 5.0 g A-5 (66.5%) as a pale yellow solid. HPLC Purity: 99.77%.

As an alternative method of conducting Step 4.1, the following conditions can be used to effect polymorph conversion. To a clean and dry, 100-ml, three-necked RBF was added A-4 (2.0 g, 0.004 mol), THF (29 ml) and DM water (1.7 ml) at room temperature. Then, maleic acid (0.44 g) was added to the reaction mixture at room temperature. The reaction mixture was stirred at room temperature for 12 h. The resulting solid was filtered and vacuum dried. The wet solid was dissolved in ethanol (12 ml) at room temperature and was stirred for 24 h. The resulting solid was filtered, washed with ethanol (2.5 ml), vacuum dried to obtain A-5 (1.5 g, 60%) as a pale yellow solid. HPLC Purity: 99.91%.

Polymorph Form B was formulated with the components indicated below into 1-mg strength capsules, wherein the percentages are calculated on a weight/weight basis:

Formulation No. 401-01

| | |
|---|---|
| Compound of Structure (I) | 0.60% |
| Lactose anhydrous | 97.40% |
| Light anhydrous silicic acid | 1% |
| Magnesium stearate | 1% |

For manufacturing, a powder blend of compound of structure (I) and the indicated excipients were encapsulated into #4 hydroxypropylmethylcellulose (HPMC) capsules. The resulting capsules were immediate-release capsules.

Prior to encapsulation into the capsules, the drug product was made by direct blending via triturating the compound of structure (I) into the indicated excipients, followed by filling the capsules on a manual capsule filling machine in 100-capsule plates.

The capsules were packaged in aluminum blister packaging, with one capsule per blister and seven capsules per blister sheet. Three blisters on each sheet were left empty.

### Example 14

### A Phase II Study of Oral Compound of Structure (I) Administered Twice Daily for 21 Days to Patients with Metastatic Castrate-Resistant Prostate Cancer

This is a Phase 2, open-label, non-randomized, Simon 2-stage design study to establish the efficacy and safety of compound of structure (I) (e.g., Form B of compound of structure (I)) taken once daily for 21 days of a 28-day cycle in patients with metastatic castration-resistant prostate cancer who have progressed on frontline treatment with androgen signaling inhibitors. A biopsy sub-study in 20 patients will enable the evaluation of tissue biomarkers in a subset of patients.

Sixty (60) patients will be enrolled. Data will be used to assess efficacy, confirm safety, and explore correlative potential biomarkers.

All patients may continue to receive compound of structure (I) in 28-day cycles (21 days of active treatment) at the same dose given during Cycle 1 until they experience unacceptable toxicity or unequivocal disease progression.

Patients must meet all of the following inclusion criteria to be eligible:
1. Male patients who also have histologically or cytologically confirmed adenocarcinoma of the prostate; AND:
   h) Be castrate-resistant on treatment with androgen deprivation therapy (ADT) (or status post bilateral orchiectomy) and with testosterone levels of less than (<) 50 nanogram per deciliter (50 ng/dL, equivalent to 1.7 nmol/L); AND:
   i) Have radiographic progression according to according to Prostate Cancer Clinical Trials Working Group 3 (PCWG3) criteria, while being treated with abiraterone acetate or enzalutamide in combination with ADT
2. Be refractory to, or intolerant of, established therapy known to provide clinical benefit for their condition
3. Have one or more tumors measurable as outlined by the Response Evaluation Criteria in Solid Tumors (RECIST) v1.1
4. Willingness to undergo two (2) on-study biopsies (biopsy sub-study cohort only)
5. Have an Eastern Cooperative Oncology Group (ECOG) performance status of ≤1
6. Have a life expectancy ≥3 months
7. Be ≥18 years of age
8. Have acceptable liver function:
   a) Bilirubin ≤1.5× upper limit of normal (ULN) (unless associated with Gilbert syndrome)
   b) Aspartate aminotransferase (AST/SGOT), alanine aminotransferase (ALT/SGPT) and alkaline phosphatase ≤2.5×ULN*
      *If liver metastases are present, then 3×ULN is allowed.
9. Have acceptable renal function: calculated creatinine clearance ≥30 mL/min
10. Have acceptable hematologic status:
   a) Granulocyte ≥1500 cells/mm³
   b) Platelet count ≥100,000 (plt/mm³)
   c) Hemoglobin ≥8 g/dL
11. Have acceptable coagulation status:
   a) Prothrombin time (PT) within 1.5× normal limits
   b) Activated partial thromboplastin time (aPTT) within 1.5× normal limits
12. Be nonfertile or agree to use an adequate method of contraception. Sexually active patients and their partners must use an effective method of contraception (hormonal or barrier method of birth control; or abstinence) prior to study entry and for the duration of study participation and for at least 3 months (males) and 6 months (females) after the last study drug dose. Should a woman become pregnant or suspect she is pregnant while her partner is participating in this study, she should inform her treating physician immediately.
13. Have read and signed the Institutional Review Board (IRB)-approved informed consent form (ICF) prior to any study-related procedure. (In the event that the patient is rescreened for study participation or a protocol amendment alters the care of an ongoing patient, a new ICF must be signed.)

Patients meeting any one of these exclusion criteria will be prohibited from participating in this study:
1. History of congestive heart failure (CHF); cardiac disease, myocardial infarction within the past 6 months prior to Cycle 1/Day 1
2. Have a corrected QT interval (using Fridericia's correction formula) (QTcF) of >450 msec in men and >470 msec in women
3. Have a seizure disorder requiring anticonvulsant therapy
4. Presence of symptomatic central nervous system metastatic disease or disease that requires local therapy such as radiotherapy, surgery, or increasing dose of steroids within the prior 2 weeks
5. Have severe chronic obstructive pulmonary disease with hypoxemia (defined as resting O₂ saturation of ≤90% breathing room air)
6. Have undergone major surgery within 2 weeks prior to Cycle 1/Day 1
7. Have active, uncontrolled bacterial, viral, or fungal infections, requiring systemic therapy
8. Are pregnant or nursing
9. Received treatment with radiation therapy, surgery, chemotherapy, or investigational therapy within 28 days or 5 half-lives, whichever occurs first, prior to study entry (6 weeks for nitrosoureas or mitomycin C)
10. Are unwilling or unable to comply with procedures required in this protocol
11. Have known infection with human immunodeficiency virus, hepatitis B, or hepatitis C. Patients with history of chronic hepatitis that is currently not active are eligible
12. Have a serious nonmalignant disease (e.g., hydronephrosis, liver failure, or other conditions) that could compromise protocol objectives in the opinion of the Investigator and/or the Sponsor
13. Are currently receiving any other investigational agent
14. Have exhibited allergic reactions to a similar structural compound, biological agent, or formulation
15. Have malabsorption conditions (e.g., Crohn's disease, etc.) or have undergone significant surgery to the gastrointestinal tract that could impair absorption or that could result in short bowel syndrome with diarrhea due to malabsorption.

Enrolled patients will receive compound of structure (I) (e.g., given as a 1-mg capsule containing Formulation No. 401-01, wherein the compound of structure (I) is Form B of the compound of structure (I)), administered twice daily (BID) for the first 21 days of a 28-day cycle. Patients who successfully complete a 4-week treatment cycle without evidence of significant treatment-related toxicity or progressive disease will continue to receive treatment with the same dose and dosing schedule.

Efficacy assessments will be performed based on PCWG3-modified RECIST v1.1 guidelines, to include the assessment of objective response rate (ORR), DoR, type of response (e.g., complete remission, partial remission, stable disease), and time to progression. The ORR is defined as the percent of patients with CR or PR according to PCWG3-modified RECIST v1.1 criteria, relative to the Response Evaluable population. ORR will be summarized by number and percentage of patients meeting the definition of ORR along with the corresponding exact 95% confidence intervals.

Tolerance and toxicity of oral compound of structure (I) will be assessed through evaluation of physical examinations, vital signs, laboratory parameters, AEs including DLTs, and all causes of mortality.

Incidence rates of treatment-emergent adverse events (TEAEs) will be summarized within each dose level at the Medical Dictionary for Regulatory Activities (MedDRA) preferred term and primary system organ class levels. Similar summaries will be made for subsets of AEs such as (1) those judged by the Investigator to be related to study treatment, and (2) serious adverse events (SAEs).

Other routine safety assessments (e.g., clinical laboratory parameters and vital signs) will be summarized by compound of structure (I) dose level using mean, standard deviation, median, minimum, and maximum changes from baseline values.

PD parameters and assessment of potential tumor and peripheral blood biomarkers including, but not limited to, CDK9-related genes (including c-Myc) in biopsy and CTC samples; Phospho-AR; PhosphoRNAPol2 on biopsy and PBMC samples; serum PSA.

Blood will be collected from all patients for evaluation of compound of structure (I) pharmacodynamics and potential biomarkers. Biopsy samples will be taken at baseline (prior to dosing on Cycle 1/Day 1) and at the end of cycle two (2) in a subset of patients participating in the biopsy sub-study.

The most recent archived tumor tissue (primary and metastatic site(s), if available) will be requested from all patients to assess potential biomarkers.

All of the U.S. patents, U.S. patent application publications, U.S. patent applications, foreign patents, foreign patent applications and non-patent publications referred to in this specification or the attached Application Data Sheet are incorporated herein by reference, in their entirety to the extent not inconsistent with the present description.

From the foregoing it will be appreciated that, although specific embodiments of the disclosure have been described herein for purposes of illustration, various modifications may be made without deviating from the spirit and scope of the disclosure. Accordingly, the disclosure is not limited except as by the appended claims.

1. A method of treating castration-resistant prostate cancer or inhibiting progression of castration-resistant prostate cancer or inhibiting proliferation of castration-resistant prostate cancer in a subject in need thereof, the method comprising administering to the subject an effective amount of crystalline Form B of a compound having the following structure (II):

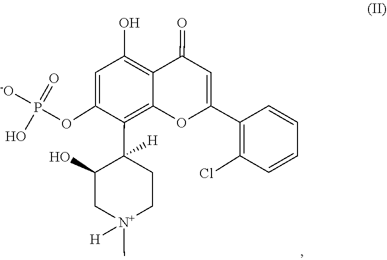

2-3. (canceled)

4. A method of treating castration-sensitive prostate cancer or inhibiting progression of castration-sensitive prostate cancer or inhibiting proliferation of castration-sensitive prostate cancer in a subject in need thereof, the method comprising administering to the subject an effective amount of a compound having the following structure (I): 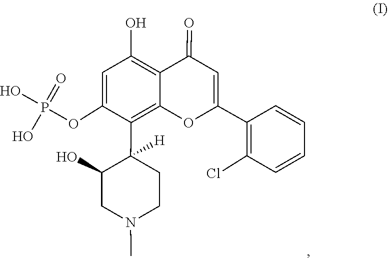 [SMILES: CN1CC[C@H](c2c(OP(=O)(O)O)cc(O)c3c(=O)cc(-c4ccccc4Cl)oc23)[C@H](O)C1]
or a pharmaceutically acceptable salt or zwitterionic form thereof.

5-6. (canceled)

7. A method of preventing or inhibiting development of castration-resistant prostate cancer in a subject having prostate cancer, the method comprising administering to the subject an effective amount of a compound having the following structure (I): 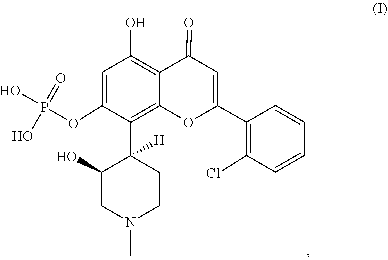 [SMILES: CN1CC[C@H](c2c(OP(=O)(O)O)cc(O)c3c(=O)cc(-c4ccccc4Cl)oc23)[C@H](O)C1]
or a pharmaceutically acceptable salt or zwitterionic form thereof.

8-11. (canceled)

12. The method of claim 1, wherein the prostate cancer is metastatic.

13. (canceled)

14. The method of claim 1, wherein from about 1 mg per day to about 60 mg per day of crystalline Form B of the compound having structure (II) is administered to the subject.

15. The method of claim 1, wherein the administering comprises orally administering.

16. (canceled)

17. The method of claim 1, wherein crystalline Form B of the compound having structure (II) is administered as a subsequent therapy after a prior therapy.

18. The method of claim 17, wherein the prior therapy comprises an androgen receptor signaling inhibitor or taxane.

19. The method of claim 18, wherein the androgen receptor signaling inhibitor is enzalutamide, apalutamide or abiraterone.

20-37. (canceled)

38. The method of claim 1, wherein crystalline Form B of the compound having structure (II) is characterized by an x-ray powder diffraction pattern comprising at least three peaks at 2-theta angles selected from the group consisting of 4.8±0.2°, 10.8±0.2°, 13.7±0.2°, 14.9±0.2°, 20.0±0.2° and 24.6±0.2°.

39-40. (canceled)

41. The method of claim 1, wherein crystalline Form B of the compound having structure (II) is characterized by an x-ray powder diffraction pattern comprising peaks at the following 2-theta angles: 10.8±0.2°, 14.9±0.2° and 20.0±0.2°.

42-43. (canceled)

44. The method of claim 1, wherein crystalline Form B of the compound having structure (II) is characterized by an x-ray powder diffraction pattern substantially in accordance with that depicted in FIG. 25.

45. (canceled)

46. The method of claim 1, further comprising administering to the subject one or more additional therapies.

47-55. (canceled)

56. The method of claim 1, wherein the castration-resistant prostate cancer is metastatic castration-resistant prostate cancer, and the subject failed a prior therapy comprising an androgen receptor signaling inhibitor or a taxane.

57-63. (canceled)

64. The method of claim 1, wherein from about 10 mg per day to about 50 mg per day of crystalline Form B of the compound having structure (II) is administered to the subject.

65-69. (canceled)

70. The method of claim 1, wherein crystalline Form B of the compound having structure (II) is administered on the first 21 days of a 28-day treatment cycle, and is not administered on days 22 to 28 of the 28-day treatment cycle.

71. The method of claim 1, wherein crystalline Form B of the compound having structure (II) is administered twice per day.

72. (canceled)

73. The method of claim 1, wherein:
(i) about 8 mg or about 11 mg of crystalline Form B of the compound having structure (II) is administered to the subject twice per day; or
(ii) about 16 mg or about 22 mg of crystalline Form B of the compound having structure (II) is administered to the subject once per day.

74. The method of claim 1, wherein crystalline Form B of the compound having structure (II) is administered on the first 14 days of a 21-day treatment cycle, and is not administered on days 15 to 21 of the 21-day treatment cycle.

75. The method of claim 1, wherein crystalline Form B of the compound having structure (II) is administered continuously.
